(19) United States
(12) Patent Application Publication
(10) Pub. No.: US 2025/0243536 A1
(43) Pub. Date: Jul. 31, 2025

(54) USE OF NANOPORE SEQUENCING FOR DETERMINING THE ORIGIN OF CIRCULATING DNA

(71) Applicant: BELGIAN VOLITION SRL, Isnes (BE)

(72) Inventors: Benjamin P. BERMAN, Jerusalem (IL); Efrat KATSMAN, Jerusalem (IL); Shari ORLANSKI, Jerusalem (IL); Silvestro CONTICELLO, Firenze (IT); Filippo MARTIGNANO, Meldola (IT); Uday MUNAGALA, Florence (IT); Amir EDEN, Jerusalem (IL)

(21) Appl. No.: 18/702,254

(22) PCT Filed: Oct. 18, 2022
(86) PCT No.: PCT/IL2022/051103

§ 371 Date: Apr. 17, 2024

Related U.S. Application Data
(60) Provisional application No. 63/256,655, filed on Oct. 18, 2021.

(51) Int. Cl. C12Q 1/6869; C12Q 1/6806; C12Q 1/6855; C12Q 1/6886; G16B 40/10

(52) U.S. Cl. CPC C12Q 1/6869; C12Q 1/6806; C12Q 1/6855; C12Q 1/6886; G16B 40/10; C12Q 2600/154

(57) ABSTRACT

Methods of determining a tissue of origin, cell type of origin, origination from a cancerous cell or a combination thereof of cell free DNA (cfDNA), comprising providing cfDNA, passing it through a nanopore sequencer to produce a sequence with DNA modification data, including DNA methylation data and DNA hydroxy methylation data, and identifying for the cfDNA the tissue of origin, cell type of origin, origination from a cancerous cell or a combination thereof based on the sequence are provided.

### CROSS-REFERENCE TO RELATED APPLICATIONS

This application claims the benefit of priority of U.S. Provisional Patent Application No. 63/256,655 filed Oct. 18, 2021, the contents of which are incorporated herein by reference in their entirety.

### FIELD OF INVENTION

The present invention is in the field of circulating DNA diagnostics and nanopore sequencing.

### BACKGROUND OF THE INVENTION

Cell-free DNA captures informative features of its originating cell, which include genomic alterations, DNA modifications such as 5-methylcytosine (5mC) and 5-hydroxymethylcytosine (5hmC), fragmentation patterns due to differential DNase activities, and nucleosomal organization. One of the most promising cfDNA biomarkers for cancer is 5mC, which has been validated in a large clinical study and is now in widespread use for cancer detection. Unlike other cancer-specific cfDNA biomarkers, 5mC can detect the presence of other unusual cell types in cfDNA to detect non-cancer conditions including myocardial infarction and sepsis. Most of these studies have used bisulfite-based approaches, but immunoprecipitation-based and enzymatic techniques have also shown promising results.

Native sequencing with the Oxford Nanopore Technologies (ONT) platform is attractive for a number of reasons. First, single base pair resolution DNA methylation calling on the Nanopore platform has improved significantly in the past several years, and now achieves high concordance with the gold standard whole-genome bisulfite sequencing (WGBS) in several benchmarking studies. ONT sequencing is also rapid, with recent clinical demonstrations of end-to-end turnaround time from sample collection to DNA methylation-based classification in as little as 1-3 hours. Other benefits of ONT for clinical settings include the low buy-in cost and portable nature of the device. ONT native WGS is unique among DNA methylation sequencing approaches in that it does not require a PCR amplification step, which can bias both fragmentation patterns and uniformity of genomic coverage.

ONT sequencing has primarily been used for long-read sequencing, but recent work has shown that it can be adapted for short fragments to detect copy number alterations, where long read sequencing is not cost effective. In a recent publication, it was shown that optimizations in library construction could generate 4-20 million sequencing reads from 4 mL of plasma of healthy and cancer patients. A method of DNA methylation and hydroxymethylation analysis of cfDNA using nanopore whole-genome sequencing is greatly needed.

### SUMMARY OF THE INVENTION

The present invention provides methods of determining a tissue of origin, cell type of origin, origination from a cancerous cell and specific cancer alterations, or a combination thereof of cell free DNA (cfDNA), comprising providing cfDNA, passing it through a nanopore sequencer to produce a sequence with methylation and/or hydroxymethylation data and identifying for the cfDNA the tissue of origin, cell type of origin, origination from a cancerous cell or a combination thereof based on the sequence.

According to a first aspect, there is provided a method of determining a tissue of origin, a cell type of origin, origination from a cancerous cell, or a combination thereof of cell-free DNA (cfDNA), the method comprising:
a. providing a sample comprising cfDNA and enriched for nucleic acids between 50 and 200 nucleotides in length to produce enriched cfDNA;
b. passing the enriched cfDNA through a nanopore sequencer to produce a sequence of the cfDNA wherein the sequence comprises DNA modification data selected from: methylation data, hydroxymethylation data and both; and
c. identifying for the enriched cfDNA passed through a nanopore a tissue of origin, cell type of origin, origination from a cancerous cell or a combination thereof based on the sequence comprising DNA modification data;
thereby determining a tissue of origin, cell type of origin, origination from a cancerous cell or a combination thereof of cfDNA.

According to another aspect, there is provided a method of determining a tissue of origin, a cell type of origin, origination from a cancerous cell, or a combination thereof of cell-free DNA (cfDNA), the method comprising:
a. providing a sample comprising cfDNA;
b. passing the cfDNA through a nanopore sequencer to produce a sequence of the cfDNA wherein the sequence comprises DNA modification data selected from: methylation data, hydroxymethylation data and both; and
c. identifying for the cfDNA passed through a nanopore a tissue of origin, cell type of origin, origination from a cancerous cell or a combination thereof based on the sequence comprising DNA modification data;
thereby determining a tissue of origin, cell type of origin, origination from a cancerous cell or a combination thereof of cfDNA.

According to another aspect, there is provided a method of determining a tissue of origin, a cell type of origin, origination from a cancerous cell, or a combination thereof of cell-free DNA (cfDNA), the method comprising:
a. providing a sample comprising cfDNA and enriched for nucleic acids smaller than 200 nucleotides;
b. passing the cfDNA through a nanopore sequencer to produce a sequence of the cfDNA wherein the sequence comprises methylation data; and
c. identifying for the passed cfDNA a tissue of origin, cell type of origin, origination from a cancerous cell or a combination thereof based on the sequence comprising methylation data;
thereby determining a tissue of origin, cell type of origin, origination from a cancerous cell or a combination thereof of cfDNA.

According to another aspect, there is provided a method of determining a tissue of origin, cell type of origin, origination from a cancerous cell or a combination thereof of cell-free DNA (cfDNA), the method comprising:
a. providing a sample comprising cfDNA and enriched for nucleic acids smaller than 200 bp;
b. passing the cfDNA through a nanopore sequencer to produce a sequence of the cfDNA;
c. performing a fragmentation analysis on the cfDNA after the passing; and
d. identifying for the passed cfDNA a tissue of origin, cell type of origin, origination from a cancerous cell or a combination thereof based on the sequence and the fragmentation analysis;
thereby determining a tissue of origin, cell type of origin, origination from a cancerous cell or a combination thereof of cfDNA.

According to some embodiments, the providing comprises providing a sample from a subject and extracting cfDNA from the sample.

According to some embodiments, the sample is a bodily fluid, optionally wherein the bodily fluid is blood.

According to some embodiments, the cfDNA is unamplified after it is extracted from a sample from a subject.

According to some embodiments, the cfDNA has been modified with a sequencing adapter and optionally a nucleic acid barcode that uniquely identifies a sample from which comes the cfDNA.

According to some embodiments, the providing further comprises employing SPRI bead size exclusion to remove DNA of a size below 50 nucleotides while retaining cfDNA of a size between 50 nucleotides and 200 nucleotides.

According to some embodiments, the SPRI bead size exclusion comprises an SPRI bead to sample ratio of about 1.8:1 by volume.

According to some embodiments, enriched is as compared to cfDNA that has undergone SPRI bead size exclusion comprising an SPRI bead to sample ratio of about 0.5:1 by volume.

According to some embodiments, the nanopore sequencer is a capable of single base pair sequencing resolution and can distinguish between methylated DNA bases, hydroxymethylated DNA bases and unmethylated/unhydroxymethylated DNA bases.

According to some embodiments, the nanopore sequencer comprises an alpha-hemolysin protein pore through which the cfDNA translocates.

According to some embodiments, the nanopore sequencer is an Oxford Nanopore sequencer.

According to some embodiments, the producing a sequence comprises applying a trained machine learning model to an electrical trace produced by the cfDNA as it translocates through the nanopore, and wherein the machine learning model is trained to identify individual bases within the electrical trace.

According to some embodiments, the identifying individual bases comprises identifying modified and unmodified DNA bases.

According to some embodiments, the machine learning model is a convolutional neural network (CNN).

According to some embodiments, identification of a modified or unmodified DNA base at an informative genetic locus indicates the tissue or cell type of origin of the cfDNA.

According to some embodiments, identification of a modified or unmodified DNA base at an informative genetic locus indicates the cfDNA is from a cancerous cell.

According to some embodiments, identification of a modified or unmodified DNA base at an informative genetic locus indicates the tissue or cell type of the cancerous cell.

According to some embodiments, a plurality of cfDNA molecules from the same source is provided and passed and identification of an average hypomethylation on the cfDNA molecules as compared to control cfDNA molecules indicates the hypomethylated cfDNA is from cancerous cells.

According to some embodiments, the control cfDNA molecules are from a subject that does not suffer from cancer.

According to some embodiments, the method is a method of determining origination from a cancerous cell and further comprises identifying a cancer-specific DNA modification change in the cancerous cell.

According to some embodiments, a plurality of cfDNA molecules from the same source is provided and passed and wherein the produced sequence has an average of at least 0.15 uniquely aligned reads covering each base in the genome or at least 2 million uniquely aligned reads total.

According to some embodiments, the method further comprises performing a fragmentation analysis on the cfDNA after the passing and wherein the identifying is based on the sequence comprising methylation data and the fragmentation analysis.

According to some embodiments, the method further comprises performing a copy number analysis on the cfDNA after the passing and wherein the identifying is based on the sequence comprising DNA modification data and the copy number analysis.

According to some embodiments, the DNA methylation is 5-methylcytosine (5mC) methylation and the hydroxymethylation is 5-hydroxymethylcytosine (5hmC) hydroxymethylation.

According to some embodiments, the cfDNA has been ligated to the sequencing adapter and further comprising performing an SPRI bead cleanup step to remove unligated sequencing adapter from the cfDNA modified with a sequencing adapter, and wherein the cleanup step comprises a first SPRI bead size exclusion comprising an SPRI bead to sample ratio of about 0.5:1 by volume and a second SPRI bead size exclusion comprising an SPRI bead to sample ratio of about 1.2:1 by volume.

According to another aspect, there is provided a method of determining a tissue of origin, cell type of origin, origination from a cancerous cell or a combination thereof of cell-free DNA (cfDNA), the method comprising:
a. providing a sample comprising cfDNA and enriched for nucleic acids between 50 and 200 nucleotides in length;
b. passing the cfDNA through a nanopore sequencer to produce a sequence of the cfDNA;
c. performing a fragmentation analysis on the cfDNA after the passing; and
d. identifying for the passed cfDNA a tissue of origin, cell type of origin, origination from a cancerous cell or a combination thereof based on the sequence and the fragmentation analysis;
thereby determining a tissue of origin, cell type of origin, origination from a cancerous cell or a combination thereof of cfDNA.

According to some embodiments, the fragmentation analysis comprises fragment length analysis, fragmentation locational analysis, fragmentation-based nucleosome detection, fragment pattern analysis, fragment end motif analysis, fragment jagged end analysis, fragmentation-based DNA-binding protein binding analysis and a combination thereof.

According to some embodiments, the method further comprises performing a copy number analysis on the cfDNA after the passing and wherein the identifying is based on the sequence the fragmentation analysis and the copy number analysis.

According to some embodiments, the copy number analysis results in the detection of an oncogene amplification and further comprising administering an agent that targets the oncogene.

According to some embodiments, the method is for use in cancer detection, early cancer screening, residual disease detection, relapse detection, metastasis detection or a combination thereof in a subject in need thereof.

According to some embodiments, the method is for use in detecting cell death or release of extracellular DNA of a tissue or cell type in a subject in need thereof.

According to some embodiments, the method further comprises treating a subject that provided the cfDNA with a suitable treatment based on the tissue of origin, cell type of origin, origination from a cancerous cell, fragmentation analysis, copy number analysis, DNA modification analysis or a combination thereof of the cfDNA.

According to another aspect, there is provided a method of producing an adapter ligated cfDNA library for analysis with a nanopore apparatus, the method comprising:
a. providing a sample comprising cfDNA;
b. ligating a short adapter below 75 nucleotides in length to the cfDNA to produce adapter ligated cfDNA;
c. removing unligated adapter from the adapter ligated cfDNA by a cleanup step comprises a first SPRI bead size exclusion comprising an SPRI bead to sample ratio of about 0.5:1 by volume and a second SPRI bead size exclusion comprising an SPRI bead to sample ratio of about 1.2:1 by volume;
thereby producing an adapter ligated cfDNA library for analysis with a nanopore apparatus.

According to some embodiments, the adapter ligated cfDNA library is enriched with cfDNA molecules of a size between 50 and 200 nucleotides.

According to some embodiments, the method further comprises passing the adapter ligated cfDNA library though a nanopore sequencer apparatus to produce a sequence of the cfDNA.

According to some embodiments, the method further comprises using the produced adapter ligated cfDNA library in a method of the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1G: Estimating cell type fractions from cfNano. (1A) Non-Negative Least Squares regression was used to deconvolute cell types in healthy plasma cfDNA samples from three whole-genome DNA methylation studies. Two representative samples are shown for each study (FF8 and FF23 for the Ilana Fox-Fisher et al., “Remote Immune Processes Revealed by Immune-Derived Circulating Cell-Free DNA”, ELife 10. November (2021) study herein incorporated by reference in its entirety; and N1 and N8 for the Hoai-Nghia Nguyen et al., “Liquid Biopsy Uncovers Distinct Patterns of DNA Methylation and Copy Number Changes in NSCLC Patients with Different EGFR-TKI Resistant Mutations”, Scientific Reports 11, no. 1 (Nature Publishing Group, 2021) study herein incorporated by reference in its entirety, and BC03 and HUIl for our cfNano samples. cfNano refers to whole-genome native sequencing of cfDNA using a Nanopore sequencing device.) Each sample is downsampled from full read depth down to an average genome coverage of 0.001 (corresponding to approximately 13,000 fragments). All samples are shown in FIGS. 5-7. (1B) Deconvolution of all samples at full depth, with samples ordered within each group by epithelial cell fraction. Healthy vs. lung adenocarcinoma (LuAd) is shown as an annotation bar, as is the “on-target/off-target” status of the Hoai-Nghia Nguyen et al., “Liquid Biopsy Uncovers Distinct Patterns of DNA Methylation and Copy Number Changes in NSCLC Patients with Different EGFR-TKI Resistant Mutations”, Scientific Reports 11, no. 1 (Nature Publishing Group, 2021) samples and the source site (HU Israel vs. BC Italy) for the cfNano samples. Asterisks mark the two HU samples with coverage less than 0.2× sequence depth. Statistical significance (p-value=0.004) is shown for percent epithelial in healthy cfNano samples vs. LuAd cfNano samples. (1C) The same samples downsampled to 0.2× sequence depth. (1D) ichorCNA CNA plots for 4 representative cfNano samples, two healthys and two LuAds. (1E) Tumor Fraction estimates (TF) from four LuAd samples based on ichorCNA from cfNano and matched Illumina WGS. (1F) Two-component DNA methylation deconvolution of lung fraction using CpGs from MethAtlas purified lung epithelia samples, showing scatter plot of ichorCNA estimates vs. deconvolution estimates for all cfNano samples. Statistical significance is shown for DNA methylation estimate of healthy cfNano vs. LuAd cfNano samples (p-value=0.003). (1G) Two-component DNA methylation deconvolution of lung cancer fraction using CpGs from TCGA LuAd tumor samples, showing scatter plot of ichorCNA estimates vs. deconvolution estimates for all cfNano samples (healthy vs. LuAd p-value=0.004). Statistical significance for panels 1B, 1C, 1F, and 1G was determined by one-tailed t-test. All cfNano samples are listed in Table 1, and all WGBS samples (Fox-Fisher et al., and Nguyen et al.) are listed in Table 2.

FIGS. 2A-2D. Genomic context of DNA methylation changes detected using cfNano. (2A) Plasma cfDNA methylation levels were averaged from −1 kb to +1 kb at 5,974 pneumocyte-specific NKX2-1 transcription factor binding sites (TFBS) taken from Kai Zhang et al., “A Single-Cell Atlas of Chromatin Accessibility in the Human Genome”, Cell 184. no. 24 (2021), herein incorporated by reference in its entirety. All methylation values are fold change relative to the flanking region (region from 0.8 kb-1 kb from the TFBS). From left to right, plots show 23 healthy plasma samples from Ilana Fox-Fisher et al. and 32 healthy plasma samples from Kun Sun et al., “Plasma DNA Tissue Mapping by Genome-Wide Methylation Sequencing for Noninvasive Prenatal, Cancer, and Transplantation Assessments”, Proceedings of the National Academy of Sciences of the United States of America 112. no. 40 (2015), herein incorporated by reference in its entirety; 3 healthy and 18 LuAd WGBS samples from Hoai-Nghia Nguyen et al., “Liquid Biopsy Uncovers Distinct Patterns of DNA Methylation and Copy Number Changes in NSCLC Patients with Different EGFR-TKI Resistant Mutations”, Scientific Reports 11, no. 1 (Nature Publishing Group, 2021), herein incorporated by reference in its entirety; and 7 healthy and 6 LuAd cfNano samples from this study. (2B) Average DNA methylation across chr16p, comparing lung tissue WGBS (top) to plasma cfNano samples from this study (bottom). Reference Partially Methylated Domains (PMDs) are taken from Wanding Zhou et al., “DNA Methylation Loss in Late-Replicating Domains Is Linked to Mitotic Cell Division”, Nature Genetics 50, no. 4 (2018), herein incorporated by reference in its entirety. (2C) Methylation delta is shown for all 10Mbp bins overlapping a reference PMD (methylation delta defined as the average methylation of the bin minus the average methylation genome-wide). Each cancer sample was compared to the group of healthy samples using a one-tailed t-test, and statistical significance is shown using asterisks. (2D) 10Mbp PMD bins were stratified by copy number status for each cancer sample using ichorCNA, and statistically significant differences were calculated by performing one-tailed Wilcoxon tests within each sample. *p<0.05. **p<0.01. ***p<0.001. ****p<0.0001.

FIGS. 3A-3C. cfNano preserves nucleosome positioning signal. (3A) Alignments to 9,780 CTCF motifs within non-promoter ChIP-seq peaks were taken from Theresa K. Kelly et al., “Genome-Wide Mapping of Nucleosome Positioning and DNA Methylation within Individual DNA Molecules”, Genome Research 22, no. 12 (2012), herein incorporated by reference in its entirety. (3B) Sequence coverage of mononucleosomes (130-155 bp) from cfNano samples is shown as fold-change vs. average coverage across the genome (top). Mononucleosome coverage for matched Illumina samples (bottom). (3C) Same analysis, using a randomly selected downsampling of 2 million reads from each sample. Two cfNano samples with less than 2M reads total are omitted.

FIGS. 4A-4J. Cancer-associated fragmentation features of cfNano vs. Illumina WGS. (4A) Fragment length density plot for each cfNano sample, with cancer samples divided into low tumor fraction (TF<0.15) and high tumor fraction (TF>0.15) based on ichorCNA. Short mononucleosomes are defined as 100-150 bp and short dinucleosomes are defined at 275-325 bp. (4B) The ratio (fraction) of short mononucleosome fragments (100-150 bp) to all mononucleosome fragments (100-220 bp). (4C) Short mononucleosome ratios based on cfNano are compared to short mononucleosome ratios based on matched Illumina WGS libraries for four LuAd cases. cfNano samples were processed with either 2019 Oxford Nanopore Real-time basecalling model (2019) or 2022 Oxford Nanopore High Accuracy model (HAC), as indicated by color. (4D) The ratio (fraction) of short dinucleosome fragments (275-325 bp) to all dinucleosome fragments (275-400 bp). (4E) Short dinucleosome ratios based on cfNano vs. Illumina WGS ratios for matched LuAd samples. (4F) Frequency of 4-mer sequences occurring at fragment ends, for cfNano vs. matched Illumina samples. The 25 most frequent 4-mers are shown in ranked order based on frequencies in Rebecca W. Y. Chan et al., “Plasma DNA Profile Associated with DNASE1L3 Gene Mutations: Clinical Observations, Relationships to Nuclease Substrate Preference, and In Vivo Correction”, American Journal of Human Genetics 107, no. 5 (2020), herein incorporated by reference in its entirety. (4G) End motif frequencies for all 256 possible 4-mers, comparing average frequency in four cfNano samples vs four matched Illumina WGS samples. (4H) End motif frequencies, comparing average frequency in four healthy HU Italy cfNano samples vs three healthy HU Israel cfNano samples. (4I) Frequency of CCCA 4-mer in all cfNano samples. (4J) CCCA 4-mer frequencies from cfNano samples vs. frequencies calculated from Illumina WGS for four matched LuAd samples. Statistical significance levels for panels 4B,4D, and 4I were determined by two-tailed t-test.

FIG. 5. DNA methylation deconvolution for high coverage healthy WGBS samples. Each sample from Fox-Fisher et al. was downsampled from full depth to 0.001× coverage, and sample ordering is the same as FIG. 1B-1C. Short names are used, and full sample information is available in Table 2.

FIG. 6. DNA methylation deconvolution for healthy and lung adenocarcinoma samples from Nguyen et al., Each sample from Nguyen et al. was downsampled from full depth to 0.001× coverage, and sample ordering is the same as FIG. 1B-1C. Short names are used, and full sample information is available in Table 2.

FIG. 7. DNA methylation deconvolution for cfNano samples. Each cfNano sample from the current study was downsampled from full depth to 0.001× coverage, and sample ordering is the same as FIG. 1B-1C.

FIGS. 8A-8C. Full cell type assignments in deconvolution analysis. (8A) Cell-type deconvolution for WGBS and cfNano datasets, using 25 cell types from MethAtlas. (8B) 25 cell type deconvolution of all samples downsampled to 0.2× sequence coverage. (8C) The four cell-type groups from FIG. 1 (Lymphocyte, Granulocyte. Epithelial, and Other) and which of the 25 cell types were collapsed into each group. All cell types not assigned to one of the four groups are shown as a singleton cell type in FIG. 1.

FIG. 9. ichorCNA tumor fractions of downsampled Illumina samples. Four Illumina plasma samples from LuAd patients are shown. ichorCNA tumor fraction was computed at full sequence depth (x axis) and by randomly downsampling the Illumina samples to have the same number of fragments as the corresponding cfNano sample.

FIGS. 10A-10C. Calling cfNano methylation with two different methods. (10A) DeepSignal and Megalodon were used to call CpG methylation for each cfNano sample. CpGs were divided into those covered by DeepSignal only. Megalodon only, or Both. Those covered by both were divided into those that got identical methylation status vs. different methylation status. (10B) Grouped cell type deconvolution is shown for all samples for Megalodon and DeepSignal processed data. Megalodon version is reproduced from FIG. 1B. (10C) Two-component deconvolution is shown for all samples for Megalodon and DeepSignal processed data. Megalodon versions are reproduced from FIGS. 1F and 1G, respectively.

FIGS. 11A-11F. Genomic context of DNA methylation changes. (11A) Methylation in 18 TCGA WGBS non-lung tumors (left) and 11 TCGA WGBS lung tumors and adjacent normal tissue (right) from Zhou et al., Plasma cfDNA methylation levels were averaged from −1 kb to +1 kb relative to 5.974 pneumocyte-specific NKX2-1 transcription factor binding sites (TFBS) taken from Zhang et al., All methylation values are shown as relative to the flanking region (from 0.8 kb-1 kb relative to TFBS). (11B) 9,274 adrenal cortical cell specific KLF5 TFBS taken from Zhang et al., From left to right, plots show 23 healthy plasma samples from Fox-Fisher et al. and 32 healthy plasma samples from K. Sun et al., followed by 3 healthy and 18 LuAd WGBS samples from Nguyen et al. and 7 healthy and 6 LuAd cfNano samples from this study. (11C) cfNano methylation levels for lung NKX2-1 (same as FIG. 2A), using DeepSignal methylation calling. (11D) IGV analysis (same as FIG. 2B) using DeepSignal methylation calling. (11E-F) Genome-wide PMD bin analysis (same as FIG. 2C-D, respectively) using DeepSignal methylation calling.

FIGS. 12A-12C. cfNano preserves fragmentomic and DNA methylation markers of nucleosome positioning. Alignments to CTCF motifs within 9.780 distal ChIP-seq peaks from Kelly et al. (12A, top) cfDNA fragment coverage shown as fold-change vs. average coverage depth across the genome. The plot includes only fragments of length 130-155 bp to maximize resolution. (12A, bottom) Matched Illumina samples of higher sequencing depth (median 17.0M fragments in Illumina vs. 6.4M in ONT samples). (12B) CTCF DNA methylation of Nanopore samples from this study at CTCF sites. (12C) DNA methylation from seven lung tissue WGBS samples from TCGA Zhou et al.

FIGS. 13A-13H. Effects of downsampling on fragment length of cfNano and Illumina WGS. (13A-13C) Data from FIGS. 4A, 4B, 4D are reproduced with the addition of sample 19_326 (which used a different, non-barcoded, cfNano adapter design), as well as matched Illumina samples. (13D) Short mononucleosome ratios (x axis) plotted against short dinucleotide ratios (y axis). Panels (13E-13H) show the same plots as panels 13A-13D, but with each sample randomly downsampled to 2M fragments. Statistical significance levels for panels 13B, 13C, 13F, and 13G were determined by two-tailed t-test.

FIGS. 14A-14D. Effects of downsampling on fragment end features of cfNano and Illumina WGS. (14A-14B) are reproduced from FIGS. 4F and 4I, with the addition of sample 19_326 (which used a different, non-barcoded, cfNano adapter design), as well as matched Illumina samples. Panels (14C-14D) show the same plots, but with each sample randomly downsampled to 2M fragments.Statistical significance levels for panels 14B and 14D were determined by two-tailed t-test.

FIG. 15. Detection of cancer cell of origin at decreasing concentrations. “healthy Mix” is a pooled plasma sample that includes 11 healthy individuals screened for breast cancer with negative results, at Hadassah Medical Center. PL5655_CRC is plasma from a single metastatic colon cancer individual, also from Hadassah Medical center. “mix” samples are mixtures of “healthyMix” and PL5655_CRC plasma at specified ratios. Mix50 is a 50/50 ratio, mix25 is 25/75 ratio, mix12.5 is 12.5/87.5 ratio, mix6.25 is 6.25/93.75 ratio, mix3.125 is 3.125/96.875 ratio. All samples are described in Table 4.

FIGS. 16A-16B. Detection of ERBB2 amplifications from multiple cfNano features. (16A) ichorCNA copy number alteration analysis of two cancer plasma cfNano samples from Hadassah Medical Center. Both show high level focal amplifications overlapping the ERBB2 gene on chr17. CRC=colorectal cancer. BRCA=breast cancer. (16B) Inset of cfNano sequence coverage at the ERBB2 gene (highlighted) for the two samples above, shown in Integrated Genome Viewer (IGV). Samples are described in Table 4.

FIG. 17. Multimodal analysis of copy number and fragment length. ichorCNA copy number levels are shown for 1-megabase bins along chromosome 17 for the HU004.02 colorectal sample, highlighting one high copy number amplification at chr17q11.2 and another at the ERBB2 gene. Below, we divide all sequencing reads (fragments) mapped to chromosome 17 into equally sized bins of 5,000 fragments, from the start of chromosome 17 to the end. We map each of these fragment bins to the 1-megabase ichorCNA bin that contains the largest number of its consituent fragments. For each 5,000-fragment bin, we show a histogram of fragment counts for fragment lengths 100 to 200, displayed as a heatmap. Most 5,000-fragment bins have a peak around 167 bp, but the two bins overlapping the chr17q11.2 amplification and the three bins overlapping the ERBB2 amplification have shorter fragment lengths, consistent with the higher proportion of cancer DNA in these regions.

FIG. 18. 5-hydroxymethylcytosine profile at CTCF binding sites. cfNano data generated for 15 healthy individual samples and 5 colorectal cancer (CRC) patients at Hadassah Hebrew University Medical Center. Samples were processed using the Megalodon/Remora joint 5-hydroxymethylcytosine (5hmC) and 5-methylcytosine (5mC) model (Remora model dna_r9.4.1_e8 with 5hmc_5mc modifications) and the percentage of CpGs containing each modification were calculated using Megalodon. These were aligned to 9.780 evolutionarily conserved CTCF motifs occurring in distal ChIP-seq peaks from Kelly et al. and percentages are shown for 5mC (left) and 5hmC (right). Diagrams of standard nucleosome positions from Kelly et al. are shown.

FIG. 19. 5-hydroxymethylcytosine profile at ubiquitously active CpG Island Transcription Start Sites. cfNano data generated for 15 healthy individual samples and 5 colorectal cancer (CRC) patients at Hadassah Hebrew University Medical Center. Samples were processed using the joint 5-hydroxymethylcytosine (5hmC) and 5-methylcytosine (5mC) model (Remora model dna_r9.4.1_e8 with 5hmc_5mc modifications) and the percentage of CpGs containing each modification were calculated using Megalodon. These were aligned to 5,154 ubiquitously active transcription start sites (TSSs) from Kelly et al., and percentages are shown for 5mC (left) and 5hmC (right).

FIGS. 20A-20B: Fragmentation profiles obtained with bioanalyzer showing unligated adapters (˜130 and ˜330 bp peaks) in standard protocol cleanup (0.5×, left) and in custom double-cleanup protocol (0.5×+1.2×, right), both in (20A) high input (60 ng) and (20B) low input (16 ng) of barcoded sample conditions.

FIG. 21: Line graph showing DNA-sequencing pore ratios over the first 3 hours of sequencing. To estimate the fraction of pores occupied by the adapter-ligated DNA of interest (strand state pores), we calculated the sum_of_occupied_pores and then calculated a total_ratio of strand_state_pores/sum_of_occupied_pores for 0.5× (0.5× total_ratio=0.53). We calculated the same ratio (strand_state_pores/sum_of_occupied_pores) for each minute of the run, and divided it by the total_ratio of 0.5× (giving a standardized measured) to show the relative increase of strand_state_pores in 0.5×+1.2× compared to 0.5× in each minute across the 3 hours.

### DETAILED DESCRIPTION OF THE INVENTION

The present invention, in some embodiments, provides methods of determining a tissue of origin, cell type of origin, origination from a cancerous cell or a combination thereof of cell free DNA (cfDNA), comprising providing cfDNA, passing it through a nanopore sequencer to produce a sequence with methylation and/or hydroxymethylation data and identifying for the cfDNA the tissue of origin, cell type of origin, origination from a cancerous cell or a combination thereof based on the sequence and methylation and/or hydroxymethylation data. Methods of determining a tissue of origin, cell type of origin, origination from a cancerous cell or a combination thereof of cell free DNA (cfDNA), comprising providing cfDNA, passing it through a nanopore sequencer to produce a sequence, performing a fragmentation analysis on the cfDNA and identifying for the cfDNA the tissue of origin, cell type of origin, origination from a cancerous cell or a combination thereof based on the sequence and fragmentation analysis is also provided.

Analysis of circulating DNA is beginning to revolutionize prenatal diagnosis, the monitoring of graft rejection, tumor diagnosis and characterization and the diagnosis of many other conditions. However, a major limitation of all applications is the dependence on the presence of identifiable genetic differences between the tissue of interest and the host. It has been shown that determination of the tissue origins of circulating free DNA (cfDNA) can be carried out by analyzing tissue-specific methylation and/or hydroxymethylation markers. Further, the cancerous state can also be determined by methylation and/or hydroxymethylation analysis. However, cfDNA comprises a large quantity (greater than 70%) of small molecules (between 100-200 nucleotides) which are important for successful analysis. Nanopores are generally designed for the sequencing of much longer strands of DNA. Previous attempts at an analysis that uses a nanopore for assessing cfDNA for tissue of origin were poor with a paucity of reads produced (see Cheng et al., “Noninvasive Prenatal Testing by Nanopore Sequencing of Maternal Plasma DNA: Feasibility Assessment”, Clinical Chemistry, Volume 61. Issue 10, 1 Oct. 2015, Pages 1305-1306, herein incorporated by reference in its entirety). While this was sufficient for detecting Y chromosome cfDNA it would not be sufficient for DNA modification analysis for determining tissue of origin/cell type of origin/cancer state, focal copy number alterations such as ERBB2, etc. The improved method provided herein, retains all informative small cfDNAs which enables a successful and robust analysis. The instant application provides nanopore sequencing as a fast and cheap method of determining the methylation and/or hydroxymethylation status of cfDNA and thereby determining its origin. Further, unlike bisulfite sequencing, this method does not damage the DNA and thus is amenable to further analysis (e.g., fragmentation analysis) that can further aid in determining cfDNA origin. We call this new method “cfNano”.

By a first aspect, there is provided a method of analyzing DNA, the method comprising: providing a sample comprising DNA, and passing the DNA through a nanopore apparatus to produce a sequence of the DNA, thereby analyzing DNA.

By another aspect, there is provided a method of determining a tissue of origin of DNA, the method comprising: providing a sample comprising DNA, passing the DNA through a nanopore apparatus to produce a sequence of the DNA; and identifying for the passed DNA a tissue of origin based on the sequence, thereby determining a tissue of origin of DNA.

By another aspect, there is provided a method of determining a cell type of origin of DNA, the method comprising: providing a sample comprising DNA, passing the DNA through a nanopore apparatus to produce a sequence of the DNA; and identifying for the passed DNA a cell type of origin based on the sequence, thereby determining a cell type of origin of DNA.

By another aspect, there is provided a method of determining origination of DNA from a cancerous cell, the method comprising: providing a sample comprising DNA, passing the DNA through a nanopore apparatus to produce a sequence of the DNA; and identifying for the passed DNA if the DNA originated from a cancerous cell based on the sequence, thereby determining origination of DNA from a cancerous cell.

By another aspect, there is provided a method of determining a tissue of origin of DNA, the method comprising: providing a sample comprising DNA, passing the DNA through a nanopore apparatus to produce a sequence of the DNA; performing a fragmentation analysis on the DNA; and identifying for the passed DNA a tissue of origin based on the sequence and fragmentation analysis, thereby determining a tissue of origin of DNA.

By another aspect, there is provided a method of determining a cell type of origin of DNA, the method comprising: providing a sample comprising DNA, passing the DNA through a nanopore apparatus to produce a sequence of the DNA; performing a fragmentation analysis on the DNA; and identifying for the passed DNA a cell type of origin based on the sequence and fragmentation analysis, thereby determining a cell type of origin of DNA.

By another aspect, there is provided a method of determining origination of DNA from a cancerous cell, the method comprising: providing a sample comprising DNA, passing the DNA through a nanopore apparatus to produce a sequence of the DNA; performing a fragmentation analysis on the DNA; and identifying for the passed DNA if the DNA originated from a cancerous cell based on the sequence and fragmentation analysis, thereby determining origination of DNA from a cancerous cell.

In some embodiments, the method is a method of determining a tissue of origin of the cfDNA. In some embodiments, the method is a method of determining a cell type of origin of the cfDNA. In some embodiments, the method is a method of determining origination of the cfDNA from a cancerous cell. In some embodiments, the method is a method of detecting a DNA amplification. In some embodiments, the method is a method of detecting a DNA deletion. In some embodiments, the DNA is genomic DNA. In some embodiments, determining origination is determining if the cfDNA originated from a cancerous cell. In some embodiments, the determining is based on the sequence. In some embodiments, the cell type is determined based on the sequence. In some embodiments, the tissue is determined based on the sequence. In some embodiments, origination from a cancerous cell is determined based on the sequence. In some embodiments, the method is a method of detecting cancer in a subject. In some embodiments, the determining origination from a cancerous cell is detecting cancer in a subject.

In some embodiments, the method is a method of identifying a cancer-specific DNA modification in a cancer cell. In some embodiments, the method is a method of determining origination of cfDNA from a cancerous cell and further identifying a cancer-specific DNA modification in the cancerous cell. In some embodiments, the DNA modification is DNA methylation. In some embodiments, DNA modification is DNA hydroxymethylation. In some embodiments, DNA modification is DNA modification and DNA hydroxymethylation. In some embodiments, DNA methylation is 5′-methylcytosine modification. In some embodiments, DNA hydroxymethylation is 5′-hydroxymethylcytosine modification. In some embodiments, a cancer specific modification is a change in a cancer cell as compared to a non-cancerous cell. In some embodiments, the DNA modification data is cancer-specific DNA modification change. In some embodiments, the methylation data is the cancer-specific methylation change. In some embodiments, the hydroxymethylation data is the cancer-specific hydroxymethylation change. It is well known in the art that cancer-specific methylation/hydroxymethylation changes can be informative about the cancer, informing about cancer prognosis, drug efficacy and other aspects of the cancer.

In some embodiments, the method is a method of detecting amplification of an oncogene in a cancer. In some embodiments, the method is a method of determining the treatment of a subject, wherein the treatment is a treatment for a cancer originating in a specific tissue or cell type or comprising an amplification of an oncogene. In some embodiments, the treatment targets the oncogene that is amplified. In some embodiments, the method is a method of detecting cancer metastasis.

In some embodiments, the method is an in vitro method. In some embodiments, the method is an ex vivo method. In some embodiments, the method is a diagnostic method. In some embodiments, the method is a non-invasive method. In some embodiments, the method is for detection of cancer. In some embodiments, detection of DNA molecules from a cancerous cell indicates the presence of cancer in the subject that provided the sample. In some embodiments, the method is for use in cancer detection. In some embodiments, the cancer detection is early cancer detection. In some embodiments, the method is a screening method. In some embodiments, the method is a method of early cancer screening. In some embodiments, the method is for residual disease detection. In some embodiments, the method is a method of metastasis detection. In some embodiments, the metastasis detection is determining the tissue/cell type of metastasis. In some embodiments, the disease is cancer. In some embodiments, the method is for relapse detection. In some embodiments, the method is for relapse screening. In some embodiments, relapse is cancer relapse. In some embodiments, the method is for detecting cell death of a tissue in a subject in need thereof. In some embodiments, the method is for detecting cell death of a cell type in a subject in need thereof. It is well known that death of particular tissues or cell types can be indicative of specific diseases. For example, death of heart cells can indicated ischemia, heart attack or other cardiac conditions, pancreatic cell death can indicate diabetes, death of lymphocytes can indicate sepsis, death of neutrophils can indicate sepsis or severe lung infection (e.g., SAR-COV-2) and death of brain cells can indicate neurological disease. Thus, the death of a particular tissue or cell type by a method of the invention can be used for a wide range of disease diagnostics. In some embodiments, the treatment is a suitable treatment for the disease diagnosed based on the cell death. Thus, for example, if a cardiovascular disease is diagnosed a cardiovascular therapy would be provided, diabetes is diagnosed insulin is provided and so on. Additional tests may be performed to confirm the diagnosis or to find out the specific disease (e.g., finding out what cardiovascular disease is present) and then the correct treatment may be selected. Similarly, the cancer treatment can be a suitable treatment for a specific type of cancer (e.g., treatment for lung cancer vs. colorectal cancer vs. pancreatic cancer) or a suitable treatment for a metastasis to a new organ.

In some embodiments, the sample is from a subject. In some embodiments, the subject is a subject in need of a method of the invention. In some embodiments, the method is for diagnosing cancer in a subject. In some embodiments, the method is for detecting cancer in a subject. In some embodiments, the detection is early detection. In some embodiments, the detection is detection with increases sensitivity. In some embodiments, the detection is detection with increased specificity. In some embodiments, the increase is as compared to cancer detection by bisulfite sequencing. In some embodiments, bisulfite sequence is any method that comprises bisulfite sequencing for determining methylation data. In some embodiments, the increase is as compared to any other method of cancer detection other than that of the invention. In some embodiments, the detection is detection of a tumor smaller than 10 cubic cm. In some embodiments, the detection is detection of less than 0.1% tumor DNA in a cfDNA sample. In some embodiments, the detection is detection of less than 1, 0.5, 0.1, 0.05, 0.01, 0.005 or 0.001% tumor DNA in a cfDNA sample. Each possibility represents a separate embodiment of the invention. In some embodiments, the method is for detecting residual disease in a subject. In some embodiments, the disease is cancer. In some embodiments, the method is for detecting death of cancer cells in a subject. In some embodiments, the method is for detecting death of healthy cell adjacent to cancer cells in a subject. In some embodiments, the method is for monitoring metastasis. In some embodiments, the method is for monitoring disease progression in a subject. In some embodiments, progression comprises metastasis. In some embodiments, the method is for monitoring treatment efficacy in a subject. In some embodiments, increase cancer cell death indicates increased efficacy of a treatment. In some embodiments, absence or decrease in cancer cell cfDNA indicates efficacy of a treatment.

In some embodiments, the method further comprises treating the cancer. In some embodiments, the method further comprises treating the detected cancer. In some embodiments, the method further comprises treating the metastasis. In some embodiments, the method further comprises treating a subject that provided the DNA. In some embodiments, the method further comprises treating a subject that provided the sample. In some embodiments, the treating is administering an anticancer therapy. In some embodiments, the treating is reinitiated a discontinued therapy. In some embodiments, the reinitiating is after discovery of residual disease after an effective therapy. In some embodiments, the treating is with a suitable treatment. In some embodiments, suitability is determined based on the tissue or cell type of origin of the DNA. In some embodiments, the treating is continuing a treatment found to effective by a method of the invention. In some embodiments, the therapy is radiation. In some embodiments, the therapy is chemotherapy. In some embodiments, the therapy is immunotherapy. Any anti-cancer therapy known in the art may be used.

In some embodiments, the nanopore apparatus is a nanopore sequencer. In some embodiments, the nanopore apparatus comprises an array of nanopores. In some embodiments, the nanopore apparatus comprises a membrane separating an input chamber from an output chamber and a nanopore is in the membrane and produces a fluidic connection between the input and output chambers. In some embodiments, the chambers contain fluid. In some embodiments, the fluid allows ionic flow from the input chamber to the output chamber. In some embodiments, the cfDNA is placed in the input chamber. In some embodiments, the cfDNA must translocate a nanopore to reach the output chamber. In some embodiments, the membrane comprises an array of nanopores. In some embodiments, each nanopore is capable of sequencing a DNA strand as it translocates. Nanopore apparatuses and in particular nanopore sequencers are well known in the art and any such apparatus may be used.

In some embodiments, the DNA is cell-free DNA (cfDNA). In some embodiments, the sample comprises DNA. In some embodiments, the sample is devoid of cells. In some embodiments, the sample is depleted of cells. In some embodiments, the sample comprises cell free DNA. In some embodiments, the DNA is single stranded DNA (ssDNA). In some embodiments, the DNA is double stranded DNA (dsDNA). In some embodiments, the dsDNA is unzipped by the nanopore and translocates as ssDNA. In some embodiments, the DNA is sheared DNA. In some embodiments, the DNA is fragmented DNA. In some embodiments, the DNA is caspase cleaved DNA. In some embodiments, the DNA comprises an epigenetic modification. In some embodiments, the DNA is modified DNA. In some embodiments, the modification is a modification to a base of the DNA. In some embodiments, the DNA is methylated. In some embodiments, the DNA is hydroxymethylated. In some embodiments, the DNA comprises a methylated cytosine. In some embodiments, the DNA comprises a hydroxymethylated cytosine. In some embodiments, the sample comprises lysed cells. In some embodiments, the sample comprises apoptotic cells. In some embodiments, the sample comprises dead cells. In some embodiments, the sample comprises necrotic cells. In some embodiments, the sample is a blood sample. In some embodiments, the sample is a plasma sample. In some embodiments, the sample is a serum sample. In some embodiments, the sample is a bodily fluid sample. In some embodiments, the sample is a bodily fluid sample, and the DNA is cfDNA. In some embodiments, the cfDNA is circulating tumor DNA (ctDNA). In some embodiments, the sample is an enriched sample. In some embodiments, the sample is a purified sample.

In some embodiments, the sample retains the distribution of cfDNA sizes found in blood. In some embodiments, the sample retains the distribution of cfDNA sizes found in a sample provided by a subject. In some embodiments, the sample retains at least 80, 85, 90, 92, 95, 97, 99 or 100% of the cfDNA molecules from the original fluid sample. Each possibility represents a separate embodiment of the invention. In some embodiments, retains comprises at least 80, 85, 90, 92, 95, 97, 99 or 100% retention of cfDNA molecules. Each possibility represents a separate embodiment of the invention. In some embodiments, at least 85% of cfDNA molecules are retained. In some embodiments, at least 90% of cfDNA molecules are retained. In some embodiments, at least 95% of cfDNA molecules are retained. In some embodiments, retained molecules are molecules large than 50 nucleotides. In some embodiments, retained molecules are molecules large than 100 nucleotides. In some embodiments, the sample retains DNA molecules from 50-200 base-pairs in length. In some embodiments, the sample is not depleted of DNA molecules from 50-200 base-pairs in length. In some embodiments, the sample retains DNA molecules from 100-200 base-pairs in length. In some embodiments, the sample is not depleted of DNA molecules from 100-200 base-pairs in length. In some embodiments. DNA molecules from 50-200 nucleotides in length make up the same or a greater proportion of all DNA in the sample as found in blood or a fluid sample from a subject. In some embodiments, DNA molecules from 100-200 nucleotides in length make up the same or a greater proportion of all DNA in the sample as found in blood or a fluid sample from a subject.

In some embodiments, the sample is enriched for small DNA molecules. In some embodiments, small is smaller than 1000, 950, 900, 850, 800, 750, 700, 650, 600, 550, 500, 450, 400, 350, 300, 290, 280, 275, 270, 260, 250, 240, 230, 225, 220, 215, 210, 205, 200, 195, 190, 185, 180, 175, 170, 169, 168, 167, 166, 165, 160, 155 or 150 nucleotides. Each possibility represents a separate embodiment of the invention. In some embodiments, small is less than 500 nucleotides. In some embodiments, small is less than 220 nucleotides. In some embodiments, small is less than 200 nucleotides. In some embodiments, small is less than 169 nucleotides. In some embodiments, small is bigger than 1, 2, 3, 4, 5, 6, 7, 8, 9, 10, 12, 15, 17, 20, 25, 30, 35, 40, 45, 50, 60, 70, 75, 80, 90 or 100 nucleotides. Each possibility represents a separate embodiment of the invention. In some embodiments, small is bigger than 50 nucleotides. In some embodiments, small is bigger than 100 nucleotides. In some embodiments, nucleotides are base-pairs. In some embodiments, the sample is enriched for DNA molecules from 50-200 base-pairs in length. In some embodiments, the sample is enriched for DNA molecules from 100-200 base-pairs in length.

As used herein, the term “enriched” refers to a composition with an increased number of molecules as compared to a control composition. In some embodiments, enrichment occurs after end repair of the cfDNA. In some embodiments, enrichment occurs after ligation of an adapter or barcode to the cfDNA. In some embodiments, the control composition is a composition that has undergone no size exclusion. In some embodiments, the control composition is a composition that has undergone size exclusion with SPRI beads at a concentration of at most 1.5×, 1.4×, 1.3×, 1.2×, 1.1×, 1×, 0.9×, 0.8×, 0.7×, 0.6× or 0.5×, where × is the ratio of SPRI bead solution to DNA containing solution by volume. Each possibility represents a separate embodiment of the invention. In some embodiments, the control composition is a composition that has undergone size exclusion with SPRI beads at a concentration of at most 1.5×. In some embodiments, the control composition is a composition that has undergone size exclusion with SPRI beads at a concentration of at most 0.5×. In some embodiments, the control composition is a composition that has undergone size exclusion with SPRI beads at a concentration of at about 0.5×. In some embodiments, enriched is comprising small DNA molecules. In some embodiments, enriched is comprising small DNA molecules as a percentage of the total cfDNA molecules that is at least as high as in the cfDNA sample before enrichment. In some embodiments, enriched is comprising small DNA molecules as a greater percentage of the total cfDNA molecules as compared to the percentage in the cfDNA sample before enrichment. In some embodiments, the control composition is genomic DNA. In some embodiments, the control composition is all cfDNA in a given volume of fluid.

In some embodiments, the method comprises a size selection step. In some embodiments, the sample is size selected. In some embodiments, size selection is selection for small DNAs. In some embodiments, the size selection is selection for all DNAs that are larger than very small DNAs. In some embodiments, the size selection is selection for all DNAs that are larger than 50 nucleotides. In some embodiments, the size selection is selection for all DNAs that are larger than 100 nucleotides. In some embodiments, the size selection is SPRI bead size selection. In some embodiments, SPRI selection is SPRI bead size exclusion. SPRI beads are well known in the art and can be used to isolate DNA. By altering the concentration of SPRI beads one can alter the size of DNA that tends to bind. Increased numbers of beads lead to binding of smaller DNAs and fewer beads lead to preferential binding of larger DNAs. In some embodiments, the concentration of SPRI beads is increased. In some embodiments, increased is as compared to a standard protocol. In some embodiments, the ratio of bead to sample is increased. In some embodiments, the ratio is by volume. In some embodiments, the ratio of bead to sample is at least 1:1, 1.1:1, 1.2:1, 1.25:1, 1.3:1, 1.4:1, 1.5:1, 1.6:1, 1.7:1, 1.75:1, 1.8:1, 1.9:1 or 2:1. Each possibility represents a separate embodiment of the invention. In some embodiments, the ratio of bead to sample is at least 1.8:1. In some embodiments, the ratio of bead to sample is at least 1.6:1. In some embodiments, the ratio of bead to sample is at least 1.5:1. In some embodiments, the ratio of bead to sample is about 1.8:1. In some embodiments, the ratio of bead to sample is at most 1.8:1, 1.9:1, 2:1, 2.1:1, 2.2:1, 2.25:1, 2.3:1, 2.4:1, 2.5:1, 2.6:1, 2.7:1, 2.75:1, 2.8:1, 2.9:1, 3:1, 3.5:1, 4:1, 4.5:1, 5:1. Each possibility represents a separate embodiment of the invention. In some embodiments, the ratio of bead to sample is more than 1.5:1. In some embodiments, the ratio of bead to sample is between 1.5:1 and 1.8:1. In some embodiments, the ratio of bead to sample is between 1.6:1 and 1.8:1. In some embodiments, the ratio of bead to sample is between 1.7:1 and 1.8:1. In some embodiments, the ratio of bead to sample is between 1.5:1 and 1.8:1, 1.5:1 and 1.9:1, 1.5:1 and 2:1, 1.5:1 and 2.1:1, 1.6:1 and 1.8:1, 1.6:1 and 1.9:1, 1.6:1 and 2:1, 1.6:1 and 2.1:1, 1.7:1 and 1.8:1, 1.7:1 and 1.9:1, 1.7:1 and 2:1, 1.7:1 and 2.1:1, 1.8:1 and 1.9:1, 1.8:1 and 2:1, or 1.8:1 and 2.1:1. Each possibility represents a separate embodiment of the invention. In some embodiments, the ratio of bead to sample is between 1.7:1 and 1.9:1. In some embodiments, SPRI bead size exclusion removes very small DNA while retaining small DNA. In some embodiments, SPRI bead size exclusion removes DNA below 50 nucleotides while retaining DNA between 50 and 200 nucleotides. In some embodiments, SPRI bead size exclusion removes DNA below 100 nucleotides while retaining DNA between 100 and 200 nucleotides. It will be understood by a skilled artisan that larger molecules are of course also retained. In some embodiments, the SPRI bead step removes reagents from previous reactions. In some embodiments, the SPRI bead step removes the reagents without affecting the size composition of cfDNA. In some embodiments, size composition is size distribution.

In some embodiments, the sample is from a subject. In some embodiments, the subject is a mammal. In some embodiments, the mammal is a human. In some embodiments, the subject is at risk for developing cancer. In some embodiments, the subject is suspected of having cancer. In some embodiments, the subject is genetically predisposed to cancer. In some embodiments, the subject has a growth of unknown character. In some embodiments, the growth has unknown malignancy. In some embodiments, the growth in not known to be benign. In some embodiments, the subject is a healthy subject. In some embodiments, the subject is providing a routine blood sample. In some embodiments, the subject is already diagnosed with cancer by means other than those of the present invention. In some embodiments, the cancer diagnosed subject has begun cancer treatment. In some embodiments, the subject has cancer. In some embodiments, the subject is undergoing cancer treatment. In some embodiments, the subject has cancer that is in remission. In some embodiments, the subject had cancer that has been cured. In some embodiments, the subject had cancer which is now undetectable. In some embodiments, the subject has completed a regimen of cancer treatment. In some embodiments, the subject is at risk for cancer return. In some embodiments, the subject is at risk for cancer relapse.

As used herein, the term “cancer” refers to any disease characterized by abnormal cell growth. In some embodiments, cancer is further characterized by the potential or ability to invade to other parts of the body beyond the part where the abnormal cell growth originated. In some embodiments, cancer is selected from breast cancer, cervical cancer, endocervical cancer, colon cancer, lymphoma (e.g., Non-Hodgkin Lymphoma), esophageal cancer, brain cancer, head and neck cancer, renal cancer, meningeal cancer, glioma, glioblastoma, Langerhans cell cancer, lung cancer, mesothelioma, ovarian cancer, pancreatic cancer, neuroendocrine cancer, prostate cancer, skin cancer, stomach cancer, tenosynovial cancer, tongue cancer, thyroid cancer, uterine cancer, and testicular cancer. In some embodiments, the cancer is lung cancer. In some embodiments, the cancer is a solid cancer. In some embodiments, the cancer is a blood cancer. In some embodiments, the cancer is Non-Hodgkin Lymphoma. In some embodiments, the cancer is a tumor. In some embodiments, the cancer is a cancer with a known epigenetic pattern of at least one locus. In some embodiments, the cancer is a cancer with a known methylation pattern of at least one locus. In some embodiments, the cancer is a cancer that can be identified by epigenetic analysis. In some embodiments, the cancer is a cancer that can be identified by methylation analysis. In some embodiments, the cancer is a cancer that can be identified by hydroxymethylation analysis. In some embodiments, the cancer is a cancer that can be identified by fragmentation analysis.

In some embodiments of the invention, the cell type is selected from the group consisting of a pancreatic beta cell, a pancreatic exocrine cell, a hepatocyte, a brain cell, a lung cell, a uterus cell, a kidney cell, a breast cell, an adipocyte, a colon cell, a rectum cell, a cardiomyocyte, a skeletal muscle cell, a prostate cell and a thyroid cell. In some embodiments of the invention, the tissue is selected from the group consisting of pancreatic tissue, liver tissue, lung tissue, brain tissue, uterus tissue, renal tissue, breast tissue, fat, colon tissue, rectum tissue, heart tissue, skeletal muscle tissue, prostate tissue and thyroid tissue. It will be appreciated that the method is appropriate for examining if the investigated DNA is derived from a particular cell type or tissue type since the sequences analyzed are specific for particular cell/tissue types. Further, the methylation/hydroxymethylation data and/or methylation/hydroxymethylation pattern may be specific for particular cell/tissue types. Thus, for example if one wishes to determine if the DNA present in a sample is derived from pancreatic beta cells, one needs to analyze sequences which have a methylation/hydroxymethylation pattern characteristic of pancreatic beta cells.

In some embodiments, epigenetic analysis comprises determining epigenetic data. As used herein, the term “epigenetic data” refers to the information of the epigenetic status or modification of a portion of bases in the DNA molecule. In some embodiments, epigenetic data is data on an epigenetic modification. In some embodiments, epigenetic data is data on a DNA modification. In some embodiments, an epigenetic modification is an epigenetically modified base. In some embodiments, epigenetic data is methylation data. In some embodiments, epigenetic analysis is analysis of at least one mark or modification on DNA. In some embodiments, the epigenetic modification is methylation. In some embodiments, the epigenetic modification is hydroxymethylation. In some embodiments, the epigenetic modification is carboxylation. In some embodiments, the epigenetic modification is formylation. In some embodiments, the epigenetic modification is modification of a cytosine. In some embodiments, the 5′ position on the cytosine is modified. In some embodiments, the methylation is adenine methylation. In some embodiments, the epigenetic modification is methylcytosine. In some embodiments, the epigenetic modification is hydroxymethylcytosine. In some embodiments, the epigenetic modification is carboxylcytosine. In some embodiments, the epigenetic modification is formylcytosine. In some embodiments, the epigenetic modification is methyladenine.

As used herein, the term “DNA modification data” refers to methylation data, hydroxymethylation data, or both. As used herein, the term “methylation data” refers to the information of the methylation status of a portion of the bases in a DNA molecule. As used herein, the term “hydroxymethylation data” refers to the information of the hydroxymethylation status of a portion of the bases in a DNA molecule. In some embodiments, a portion is all of the bases. In some embodiments, the bases are cytosines. In some embodiments, a portion is at least 10, 20, 25, 30, 40, 50, 55, 60, 65, 70, 75, 80, 85, 90, 92, 95, 97, 99 or 100% of the bases. As used herein, the term “DNA modification status” refers to the status of a base in a DNA sequence as either methylated, hydroxymethylated or unmodified by methylation or hydroxymethylation. As used herein, the term “methylation status” refers to the status of a base in a DNA sequence as either methylated or unmethylated. As used herein, the term “hydroxymethylation status” refers to the status of a base in a DNA sequence as either hydroxymethylated or unhydroxymethylated (e.g., non-hydroxymethylated). For example, a cytosine may be methylated (and present as 5-methylcytosine), hydroxymethyalted (and present as 5′-hydroxymethylcytosine) or non-methylated and present as cytosine. As used herein, “cytosine methylation”, “methylated cytosine” and “methylcytosine” are used interchangeably and refer to a cytosine base with a methyl group covalently bonded at the 5-carbon position. As used herein, “cytosine hydroxymethylation”, “hydroxymethylated cytosine” and “hydroxymethylcytosine” are used interchangeably and refer to a cytosine base with a hydroxymethyl group covalently bonded at the 5-carbon position. In some embodiments, methylcytosine is 5-methylcytosine. In some embodiments, the cytosine is a cytosine of a CpG dinucleotide. In some embodiments, the cytosine is a cytosine of a CpG island. In some embodiments, hydroxymethylcytosine is 5-hydroxymethylcytosine. In some embodiments, carboxylcytosine is 5-carboxylcytosine. In some embodiments, formylcytosine is 5-formylcytosine. In some embodiments, methyladenine is 6-methylcytosine.

In some embodiments, providing comprises provided a sample. In some embodiments, the sample comprises DNA. In some embodiments, the DNA is cfDNA. In some embodiments, the method comprises extracting DNA from the sample. In some embodiments, extracting is isolating. In some embodiments, the DNA is native DNA. In some embodiments, the DNA is unamplified after it is extracted. In some embodiments, unamplified DNA is passed through the nanopore. In some embodiments, the DNA is unmodified. In some embodiments, the DNA is not bisulfite converted. In some embodiments, the DNA is not concatemerized. In some embodiments, the sample does not comprise concatemerized data. In some embodiments, the cfDNA is not concatemerized. In some embodiments, the passing is passing of non-concatemerized DNA. In some embodiments, the sequencing is sequencing of non-concatemerized DNA. It will be understood by a skilled artisan that native adapter/barcode ligation may result in a small percentage of concatamerization, but the method does not make use of these longer molecules but rather analyzes the short cfDNAs as they are. In some embodiments, sequencing reads from a long DNA are discarded. In some embodiments, sequencing reads from a concatamerized DNA are discarded. In some embodiments, a long DNA is any DNA that is not a short DNA.

In some embodiments, the DNA is modified. In some embodiments, the modification is end repair. Methods of end repair are well known in the art and any such method may be employed. In some embodiments, the modification is an adapter. In some embodiments, the DNA is modified with a 3′ adapter. In some embodiments, modified with is ligated to. In some embodiments, the method further comprises ligating an adapter to the cfDNA. In some embodiments, the DNA is modified with a 5′ adapter. In some embodiments, the adapter is a sequencing adapter. Sequencing adapters are well known in the art and any such adapter may be used. In some embodiments, the adapter is an adapter from the SQK-LSK109 kit. In some embodiments, the adapter is conjugated to a protein. In some embodiments, the protein is a motor protein. In some embodiments, the protein is a protein for interaction with the nanopore. In some embodiments, the protein is a protein for interaction with the helicase at the nanopore. In some embodiments, the adapter is a nanopore adapter. In some embodiments, the adapter is a nanopore specific adapter. In some embodiments, the DNA is modified with a barcode. In some embodiments, the DNA is modified with a unique molecular identifier (UMI). In some embodiments, the barcode is a sample specific barcode. In some embodiments, the method is a multiplex method and comprises passing cfDNA from a plurality of samples through the nanopore sequencer. In some embodiments, cfDNAs from each sample of the plurality of samples comprise the same sample specific barcode.

In some embodiments, the barcode is a nucleic acid barcode. In some embodiments, the barcode is readable by the nanopore sequencer. As used herein, the term “barcode” refers to a moiety that uniquely identifies the DNA molecule either as a specific molecule or as part of a group of molecules (i.e., molecules from a given sample). Barcodes are well known in the art and many commercial kits are available that provide barcodes and specifically barcodes for multiplex sequencing. For example, barcodes are provided in the EXP-NBD104 and EXP-NBD114 kits to be used with SQK-LSK109 kit. The protocol for barcoding and specifically native barcoding is also well known and is provided with these kits. In some embodiments, the barcode is a native barcode. In some embodiments, the adapter is a native adapter. In some embodiments, a native adapter/barcode is an adapter/barcode that is added by ligation. In some embodiments, addition by ligation is not addition by amplification. In some embodiments, addition by ligation is not addition by reverse transcription (RT). In some embodiments, addition by ligation does not comprise amplification or RT.

In some embodiments, the method further comprises end repairing the cfDNA. In some embodiments, the method further comprises performing end repair on the cfDNA. Methods of end repair are well known in the art and any method may be used. Kits for end repair are commercially available from companies such as Thermo Fisher, NEB, Cambio and many more. Any such kit may be employed. In some embodiments, the method further comprises a cleanup step. In some embodiments, the cleanup step is after end repair of the cfDNA. In some embodiments, the cleanup is cleanup of the end repair reaction. In some embodiments, clean up comprises removal of the end repair reagents. In some embodiments, cleanup is with SPRI beads. In some embodiments, clean up comprises SPRI bead size exclusion.

In some embodiments, the cleanup step is to remove unligated adapter or barcode. In some embodiments, the cleanup step is to remove previous reagents. In some embodiments, the previous reagents are reagents for end repair. In some embodiments, the previous reagents are reagents for ligation. In some embodiments, the previous reagent is an enzyme. In some embodiments, the enzyme is a polymerase. In some embodiments, the enzyme is Klenow. In some embodiments, the enzyme is polynucleotide kinase. In some embodiments, the enzyme is a ligase. In some embodiments, the cleanup step separates unligated adapter or barcode from cfDNA ligated to adapter or barcode. In some embodiments, unligated adapter is removed. In some embodiments, unligated barcode is removed. In some embodiments, the cleanup comprises a two-step SPRI bead size exclusion. In some embodiments, the cleanup comprises a first SPRI bead size exclusion and second SPRI bead size exclusion. In some embodiments, the first SPRI bead size exclusion comprises an SPRI bead to sample ratio of about 0.5:1. In some embodiments, the first SPRI bead size exclusion comprises an SPRI bead to sample ratio of between 0.4:1 and 0.6:1. In some embodiments, the first SPRI bead size exclusion comprises an SPRI bead to sample ratio of 0.5:1 or more. In some embodiments, the second SPRI bead size exclusion comprises an SPRI bead to sample ratio of about 1.2:1. In some embodiments, the second SPRI bead size exclusion comprises a higher ratio of bead to sample than the first SPRI bead size exclusion. In some embodiments, higher is at least double. In some embodiments, about 1.2:1 is 1.1:1 to 1.3 to 1. In some embodiments, about 1.2:1 is 1:1 to 1.4 to 1. The second SPRI beads are added to just the isolated DNA in water or a salt buffer. As such, a much higher concentration of SPRI is needed so that the desired ligated DNA is not lost, but not so high that the unligated adapter is still retained.

In some embodiments, the sample is a bodily fluid. In some embodiments, the bodily fluid is selected from: blood, serum, plasma, gastric fluid, intestinal fluid, saliva, bile, tumor fluid, breast milk, urine, interstitial fluid, cerebral spinal fluid and stool.

In some embodiments, the method comprises passing the DNA through a nanopore. In some embodiments, passing is translocating. Methods of nanopore analysis are well known in the art. Briefly, into a first reservoir on a first side of a membrane containing the nanopore or an array of nanopores is deposited the sample for analysis. An electrical current is run from the first reservoir to a second reservoir on a second side of the membrane. As DNA is negatively charged, the positive pole is placed in the second reservoir, and this causes the DNA to translocate to the second reservoir via the nanopore/s. As the DNA molecule passes through the pore its size impedes the electrical current through the pore. A sensor at the pore measures the presence of the DNA and indeed distinguishes between different bases thereby reading the sequence (i.e., sequencing) the DNA. It will be understood by a skilled artisan that nanopore sequencing generally sequences only one strand of the DNA at a time (alpha-hemolysin nanopores for example displace the second strand which is sequenced separately). Although the second strand may eventually be sequenced it cannot be associated with its sister strand. This makes methylation analyses that rely on converting unmethylated or methylated cytosines into another base (e.g., bisulfite conversion) difficult to analyze. Though the sequence becomes changed, without the sister strand to indicate where a cytosine has been converted the sequence cannot always be aligned to the correct location and the methylation data may be lost. Native DNA analysis with a nanopore however suffers from no such difficulty.

In some embodiments, the nanopore is an array of nanopores. In some embodiments, the nanopore is a nanopore sequencer. In some embodiments, the nanopore sequencer comprises a sensor at the nanopore. In some embodiments, the nanopore is a solid state nanopore. In some embodiments, the nanopore is a helicase nanopore. Helicase nanopores are well known in the art and allow the passage of ssDNA for sequencing. Adapters with motor proteins conjugated thereto can be used to contact the helicase and guide the DNA strand through the nanopore for sequencing. In some embodiments, the sensor is an electrical sensor. In some embodiments, the sensor is an optical sensor. In some embodiments, the sensor is configured to detect the DNA as it passes through the nanopore. In some embodiments, the sensor is configured to detect electrical current through the nanopore. In some embodiments, detect is measure. In some embodiments, the sensor is configured to measure changes in electrical current and/or voltage through the nanopore and thereby detect the DNA. In some embodiments, the sensor is configured to measure changes in electrical current and/or voltage through the nanopore and thereby sequence the DNA. In some embodiments, sequencing is detecting the nucleotide sequence in order. In some embodiments, sequencing comprises detecting the unique change in current and/or voltage produced by each nucleotide. In some embodiments, sequencing comprises detecting the unique change in current and/or voltage produced by adenine, thymine, cytosine and guanine bases. In some embodiments, the nanopore sequencer is capable of single base pair sequencing resolution. In some embodiments, the nanopore sequencer is configured for single base pair sequencing resolution.

In some embodiments, the nanopore is a solid-state nanopore. In some embodiments, the nanopore comprises a protein pore. In some embodiments, the nanopore is a protein pore. In some embodiments, the nanopore comprises a protein at the nanopore. In some embodiments, the protein facilitates translocation of the DNA. In some embodiments, the DNA translocates though the protein pore. In some embodiments, the protein facilitates a stepwise passage of the DNA through the nanopore. In some embodiments, stepwise is a nucleotide at a time. In some embodiments, stepwise passage is a slow enough passage to allow the sensor to uniquely identify single bases. Protein nanopores are well known in the art and any such suitable protein may be used from the pore. Examples of such pore proteins include but are not limited to alpha-hemolysin, aerolysin and MspA porin. In some embodiments, the protein pore is an alpha-hemolysin pore. In some embodiments, nanopore sequencer is an Oxford Nanopore sequencer. In some embodiments, the Oxford Nanopore sequencer is a MinION sequencer. It will be understood by a skilled artisan that the exact nanopore sequencer used is not material to the invention, but rather the ability of the nanopore to produce single nucleotide resolution of the DNA as it translocates is essential. For methods that require methylation data in addition to sequencing data it is essential that the nanopore produces methylation level resolution of the nucleotide.

In some embodiments, producing a sequence comprises determining nucleotide identity from an electrical trace. In some embodiments, producing a sequence is sequencing. In some embodiments, the sequencing is whole genome sequencing (WGS). In some embodiments, the sequencing is targeted sequencing. In some embodiments, the target is a sequence of an informative locus. In some embodiments, the target is a plurality of targets. In some embodiments, the sequencing is methylation sequencing. In some embodiments, the electrical trace is produced by the DNA as it translocates through the nanopore. In some embodiments, the electrical trace is the measuring produced by the sensor. In some embodiments, the electrical trace is a current trace. In some embodiments, the electrical trace is a voltage trace. As used herein, the term “trace” refers to a continuous readout or measure of a parameter at the nanopore. In some embodiments, a trace is a readout. In some embodiments, the electrical trace comprises the change in electrical current or voltage at the nanopore as each nucleotide passes through the nanopore.

In some embodiments, the electrical trace is analyzed by applying a trained machine learning model to it. In some embodiments, the producing a sequence comprises applying a trained machine learning model to the electrical trace. In some embodiments, the machine learning model is trained to identify individual bases. In some embodiments, the individual bases are individual bases within an electrical trace. In some embodiments, the machine learning model is trained on known sequences of DNA molecules and the electrical trace they produce as they translocate through the nanopore. In some embodiments, the machine learning model is a convolutional neural network (CNN). In some embodiments, the machine learning model is the DeepSignal machine learning model. In some embodiments, the CNN is DeepSignal. Examples of CNN algorithms that can be employed in the method of the invention include, but are not limited to DeepSignal, Megalodon, DeepMod, mCaller, and Guppy. In some embodiments, the machine learning model is not a CNN. Examples of non-CNN algorithms that can be employed in the method of the invention include, but are not limited to Nanopolish, Tombo, NanoMod, SignalAlign, and methBERT.

Examples of machine learning models are well known and include for example neural networks, and classifiers which may be supervised, semi-supervised, or unsupervised as necessary for performing the method of the invention. In some embodiments, the neural network models employed by the present invention to determine DNA sequence may be selected from the group consisting of Neural Bag-of-Words (NBOW); recurrent neural network (RNN), Recursive Neural Tensor Network (RNTN); Dynamic Convolutional Neural Network (DCNN); Long short-term memory network (LSTM); recursive neural network (RecNN). And Convolutional neural network (CNN).

In some embodiments, the sequence comprises methylation data. In some embodiments, the sequence produce by the nanopore sequencer comprises methylation data. In some embodiments, the nanopore sequencer produces methylation data for the sequence. In some embodiments, the nanopore sequencer when sequencing a cytosine also determines its methylation status. In some embodiments, the method does not comprise bisulfite conversion. In some embodiments, the methyl group is measured directly. It will be understood by a skilled artisan that the addition of a methyl group to a cytosine will alter the nucleotides effect on ion flow through the nanopore. This difference in ion flow (i.e., electrical current) can be measured/detected. In some embodiments, a methylated cytosine and unmethylated cytosine are distinguishable on an electrical trace. In some embodiments, the sensor is configured to detect methylated and unmethylated cytosines. In some embodiments, the sensor comprises a sensitivity sufficient to distinguish between methylated and unmethylated cytosines. In some embodiments, the sensor is configured to detect methylated cytosine nucleotides as they pass through the nanopore. In some embodiments, the sensor is configured to detect the electrical change produced by a methylated cytosine as compared to an unmethylated cytosine as it passes through the nanopore. In some embodiments, the sensor is configured to detect the electrical change produced by a hydroxymethylated cytosine as compared to an unhydroxymethylated cytosine as it passes through the nanopore. In some embodiments, the sensor is configured to detect the electrical change produced by a methylated cytosine as compared to a hydroxymethylated cytosine as it passes through the nanopore. In some embodiments, sequencing comprises detecting the unique change in current and/or voltage produced by each nucleotide and methylated cytosine. In some embodiments, each nucleotide is adenine, thymine, unmethylated cytosine, methylated cytosine and guanine bases. In some embodiments, sequencing comprises detecting the unique change in current and/or voltage produced by adenine, thymine, unmethylated cytosine, methylated cytosine and guanine bases. In some embodiments, the nanopore sequencer is capable of single base pair methylation resolution. In some embodiments, the nanopore sequencer is configured for single base pair methylation sequencing resolution. In some embodiments, the nanopore sequencer is configured for single base pair hydroxymethylation sequencing resolution. In some embodiments, the nanopore sequencer is configured for single base pair DNA modification sequencing resolution.

In some embodiments, producing a sequence further comprises producing methylation data. In some embodiments, producing methylation data comprises determining cytosine methylation status from an electrical trace. In some embodiments, producing a sequence further comprises producing hydroxymethylation data. In some embodiments, producing methylation data comprises determining cytosine hydroxymethylation status from an electrical trace. In some embodiments, the electrical trace comprises the change in electrical current or voltage at the nanopore as a methylated cytosine passes through the nanopore. In some embodiments, the electrical trace comprises the change in electrical current or voltage at the nanopore as an unmethylated cytosine passes through the nanopore. In some embodiments, the electrical trace comprises the change in electrical current or voltage at the nanopore as a hydroxymethylated cytosine passes through the nanopore. In some embodiments, the electrical trace comprises the change in electrical current or voltage at the nanopore as an unhydroxymethylated cytosine passes through the nanopore. In some embodiments, the electrical trace comprises a difference in electrical current or voltage at the nanopore between a methylated cytosine and unmethylated cytosine passing through the nanopore. In some embodiments, the electrical trace comprises a difference in electrical current or voltage at the nanopore between a hydroxymethylated cytosine and unhydroxymethylated cytosine passing through the nanopore.

In some embodiments, producing methylation data comprises applying a trained machine learning model to the electrical trace. In some embodiments, producing DNA modification data comprises applying a trained machine learning model to the electrical trace. In some embodiments, producing hydroxymethylation data comprises applying a trained machine learning model to the electrical trace. In some embodiments, the machine learning model is trained to identify methylated and unmethylated cytosines. In some embodiments, the machine learning model is trained to identify hydroxymethylated and unhydroxymethylated cytosines. In some embodiments, the machine learning model is trained to identify modified and unmodified cytosines. In some embodiments, the machine learning model is trained to distinguish between modified and unmodified cytosines. In some embodiments, the machine learning model is trained to distinguish between methylated and unmethylated cytosines. In some embodiments, the machine learning model is trained to distinguish between hydroxymethylated and unhydroxymethylated cytosines. In some embodiments, the methylated and unmethylated cytosines are within an electrical trace. In some embodiments, the hydroxymethylated and unhydroxymethylated cytosines are within an electrical trace. In some embodiments, the machine learning model is trained on sequences with known methylation status of DNA molecules and the electrical trace they produce as they translocate through the nanopore. In some embodiments, the machine learning model is trained on sequences with known modification status of DNA molecules and the electrical trace they produce as they translocate through the nanopore. In some embodiments, the machine learning model is trained on sequences with known hydroxymethylation status of DNA molecules and the electrical trace they produce as they translocate through the nanopore. In some embodiments, the sequences with known methylation status are sequences with the methylation status of a cytosine given. In some embodiments, the sequences with known hydroxymethylation status are sequences with the hydroxymethylation status of a cytosine given. In some embodiments, a cytosine is a plurality of cytosines. In some embodiments, a cytosine is all cytosines in the sequence. In some embodiments, the DeepSignal machine learning model is as disclosed in Ni et al., “DeepSignal: detecting DNA methylation state from Nanopore sequencing reads using deep-learning”, Bioinformatics, 2019 November 1; 35 (22): 4586-4595, herein incorporated by reference in its entirety.

In some embodiments, the tissue of origin is determined based on the DNA modification data. In some embodiments, the cell type of origin is determined based on the DNA modification data. In some embodiments, origination from a cancerous cell is determined based on the DNA modification data. In some embodiments, the tissue of origin is determined based on the sequence and the DNA modification data. In some embodiments, the cell type of origin is determined based on the sequence and the DNA modification data. In some embodiments, origination from a cancerous cell is determined based on the sequence and the DNA modification data. In some embodiments, the sequence and the DNA modification data is a combination of the sequence and the DNA modification data.

In some embodiments, the tissue of origin is determined based on the methylation data. In some embodiments, the cell type of origin is determined based on the methylation data. In some embodiments, origination from a cancerous cell is determined based on the methylation data. In some embodiments, the tissue of origin is determined based on the sequence and the methylation data. In some embodiments, the cell type of origin is determined based on the sequence and the methylation data. In some embodiments, origination from a cancerous cell is determined based on the sequence and the methylation data. In some embodiments, the sequence and the methylation data is a combination of the sequence and the methylation data.

In some embodiments, the tissue of origin is determined based on the hydroxymethylation data. In some embodiments, the cell type of origin is determined based on the hydroxymethylation data. In some embodiments, origination from a cancerous cell is determined based on the hydroxymethylation data. In some embodiments, the tissue of origin is determined based on the sequence and the hydroxymethylation data. In some embodiments, the cell type of origin is determined based on the sequence and the hydroxymethylation data. In some embodiments, origination from a cancerous cell is determined based on the sequence and the hydroxymethylation data. In some embodiments, the sequence and the hydroxymethylation data is a combination of the sequence and the hydroxymethylation data.

In some embodiments, the DNA is from an informative genomic location. In some embodiments, the genomic location is a genomic locus. As used herein, the term “informative location” or “informative locus” refers to a DNA sequence whose methylation/hydroxymethylation status is informative with respect to at least one of tissue of origin, cell type of origin or origination from a cancerous cell. Although, most locations are not informative about the tissue/cell of origin or origination from cancer, there are locations well known in the art that are informative. In some embodiments, epigenetic modification at an informative genomic locus indicates the DNA is from a given tissue/cell/cancer/not cancer. In some embodiments, methylation at an informative genomic locus indicates the DNA is from a given tissue/cell/cancer/not cancer. In some embodiments, the epigenetic data at an informative genomic location is a cancer-specific epigenetic change. In some embodiments, the methylation data at an informative genomic location is a cancer-specific methylation change. In some embodiments, a genomic locus is a plurality of genomic loci. In some embodiments, a genomic locus is a combination of genomic loci. In some embodiments, the genomic locus is at least 1, 2, 3, 4, 5, 6, 7, 8, 9 or 10 loci. Each possibility represents a separate embodiment of the invention. In some embodiments, methylation is hypermethylation. In some embodiments, hypermethylation comprises at least 50, 55, 60, 65, 70, 75, 80, 85, 90, 95, 97, 99 or 100% methylation of CpGs in the informative locus. Each possibility represents a separate embodiment of the invention. In some embodiments, unmethylation at an informative genomic locus indicates the DNA is from a given tissue/cell/cancer/not cancer. In some embodiments, unmethylation is hypomethylation. In some embodiments, hypomethylation comprises at most 1, 3, 5, 7, 10, 15, 20, 25, 30, 35, 40, 45 or 50% methylation of CpGs in the informative locus. Each possibility represents a separate embodiment of the invention.

In some embodiments, methylation or unmethylation of the informative genetic locus is tissue or cell type specific. In some embodiments, methylation or unmethylation of the informative genetic locus is cancer specific. In some embodiments, methylation or unmethylation of the informative genetic locus is non-cancer specific. In some embodiments, hydroxymethylation or unhydroxymethylation of the informative genetic locus is tissue or cell type specific. In some embodiments, hydroxymethylation or unhydroxymethylation of the informative genetic locus is cancer specific. In some embodiments, hydroxymethylation or unhydroxymethylation of the informative genetic locus is non-cancer specific. In some embodiments, it is informative of the tissue or cell type in which the methylation/unmethylation occurs. In some embodiments, it is informative of the cancer state of the cell in which the methylation/unmethylation occurs. In some embodiments, it is informative of both the tissue of origin and/or cell type and the cancer state of the cell in which the methylation/unmethylation occurs. In some embodiments, it is informative of the tissue or cell type in which the hydroxymethylation/unhydroxymethylation occurs. In some embodiments, it is informative of the cancer state of the cell in which the hydroxymethylation/unhydroxymethylation occurs. In some embodiments, it is informative of both the tissue of origin and/or cell type and the cancer state of the cell in which the hydroxymethylation/unhydroxymethylation occurs. Informative loci for numerous tissues, cell types and cancer are known in the art and any such loci may be used. Informative loci may be found, for example, in International Patent Applications WO2019012542, WO2019012543, and WO2020212992 herein incorporated by reference in their entirety.

In some embodiments, identification of DNA modification at an informative genetic locus indicates the tissue of origin of the DNA. In some embodiments, identification of DNA modification at an informative genetic locus indicates the cell type of origin of the DNA. In some embodiments, identification of DNA modification at an informative genetic locus indicates the DNA originated from a cancerous cell. In some embodiments, identification of DNA modification at an informative genetic locus indicates the DNA originated from a non-cancerous cell. In some embodiments, identification of unmodification at an informative genetic locus indicates the tissue of origin of the DNA. In some embodiments, identification of unmodification at an informative genetic locus indicates the cell type of origin of the DNA. In some embodiments, identification of unmodification at an informative genetic locus indicates the DNA originated from a cancerous cell. In some embodiments, identification of unmodification at an informative genetic locus indicates the DNA originated from a non-cancerous cell. In some embodiments, DNA modification is methylation. In some embodiments, DNA modification is hydroxymethylation. n some embodiments, DNA modification is methylation and hydroxymethylation. In some embodiments, unmodification is unmethylation. In some embodiments, unmodification is unhydroxymethylation. In some embodiments, unmodification is neither methylation nor hydroxymethylation.

In some embodiments, the locus is between 2 and 20, 2 and 16, 2 and 12, 2 and 10, 2 and 8, 2 and 6, 2 and 4, 4 and 20, 4 and 16, 4 and 12, 4 and 10, 4 and 8 or 4 and 6 base pairs. Each possibility represents a separate embodiment of the invention. In some embodiments, the locus is a nucleosome, or a nucleosome length of DNA (˜170 bp). In some embodiments, the genetic locus is between 150 and 190, or 160 and 180 bp. In some embodiments, hypomethylation in the informative locus indicates the cfDNA is from cancer.

In some embodiments, a plurality of DNA molecules from the same source is provided. In some embodiments, the same source is the same sample. In some embodiments, the same source is the same subject. In some embodiments, the plurality of DNA molecules are passed through the nanopore. In some embodiments, passing comprises inducing an electrical current from one side of the nanopore to the other. In some embodiments, the electrical current is from a negative pole in a first reservoir containing the sample to a positive pole in a second reservoir on the opposite side of the nanopore.

In some embodiments, identification of hypomethylation on the DNA molecules indicates the hypomethylated DNA is from a cancerous cell. In some embodiments, the DNA molecules are the plurality of DNA molecules. In some embodiments, hypomethylation of the DNA molecules is an average hypomethylation on the plurality of molecules. In some embodiments, hypomethylation is as compared to control DNA molecules. In some embodiments, the control DNA molecules are control cfDNA molecules. In some embodiments, the control molecules are from a subject that does not suffer from cancer. In some embodiments, the control molecules are from a sample from a subject that does not suffer from cancer.

In some embodiments, the sequencing depth of the nanopore sequencer is at least a 0.2× sequencing depth. In some embodiments, the sequencing depth of the nanopore sequencer is at least a 2× sequencing depth. In some embodiments, the sequencing depth across the plurality of DNA molecules is at least a 0.2× sequencing depth. In some embodiments, the sequencing depth across the plurality of DNA molecules is at least a 2× sequencing depth. In some embodiments, the sequences produced from the plurality of molecules comprise at least a 0.2× sequencing depth. In some embodiments, the sequences produced from the plurality of molecules comprise at least a 2× sequencing depth. In some embodiments, at least a 0.2× sequencing depth is at least a 0.2×, 0.4×, 0.5×, 0.6×, 0.75×, 0.8×, 1×, 1.5×, 2×, 2.5×, 3×, 3.5×, 4×, 4.5×, 5×, 6×, 7×, 8×, 9× or 10× sequencing depth. Each possibility represents a separate embodiment of the invention. In some embodiments, at least a 0.2× sequencing depth is about 0.2× sequencing depth. In some embodiments, the produced sequences have an average of at least 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.25, 0.30, 0.35, 0.40, 0.45, or 0.50 uniquely aligned reads covering each base. Each possibility represents a separate embodiment of the invention. In some embodiments, the produced sequences have an average of at least 0.15 uniquely aligned reads covering each base. In some embodiments, each base is each base of the sample. In some embodiments, each base is each base of the DNA. In some embodiments, each base is each base of the genome. In some embodiments, each base is each base of all the produced sequences. In some embodiments, the produced sequences comprise at least 0.1, 0.25, 0.5, 0.75, 1, 1.25, 1.5, 1.75, 2, 2.25, 2.5, 2.75, 3, 3.25, 3.5, 3.75, 4, 4.5 or 5 million reads. Each possibility represents a separate embodiment of the invention. In some embodiments, the produced sequences comprise at least 2 million reads. In some embodiments, reads are unique reads. In some embodiments, unique reads are uniquely alignable reads. In some embodiments, alignable reads are reads that can be aligned with a target genome. In some embodiments, the genome is the genome of a subject. In some embodiments, an alignable read is an aligned read.

In some embodiments, the method further comprises performing an additional analysis on the DNA. In some embodiments, the additional analysis is fragmentation analysis. In some embodiments, the additional analysis is copy number analysis. In some embodiments, the copy number analysis is performed on the DNA after passing. In some embodiments, the copy number analysis is performed on the DNA after sequencing. In some embodiments, the copy number analysis produced copy number data. In some embodiments, a DNA with a known sequence undergoes copy number analysis. In some embodiments, a DNA with known modification data undergoes copy number analysis. In some embodiments, a DNA with known methylation data undergoes copy number analysis. In some embodiments, a DNA with known hydroxymethylation data undergoes copy number analysis. In some embodiments, a DNA with known fragmentation data undergoes copy number analysis.

In some embodiments, the method further comprises performing a fragmentation analysis on the DNA. In some embodiments, the fragmentation analysis is performed on the DNA after passing. In some embodiments, the fragmentation analysis is performed on the DNA after sequencing. In some embodiments, the fragmentation analysis is performed on the DNA before passing. In some embodiments, the fragmentation analysis is performed on the DNA before sequencing. In some embodiments, the DNA is fragmentated before performing passing and analyzed after passing. In some embodiments, the DNA is fragmentated before performing sequencing and analyzed after sequencing. In some embodiments, the fragmentation analysis produces fragmentation data. In some embodiments, a DNA with a known sequence undergoes fragmentation analysis. In some embodiments, a DNA with known modification data undergoes fragmentation analysis. In some embodiments, a DNA with known methylation data undergoes fragmentation analysis. In some embodiments, a DNA with known hydroxymethylation data undergoes fragmentation analysis. In some embodiments, a DNA with known copy number data undergoes fragmentation analysis.

As used herein, the term “fragmentation analysis” refers to an assay in which the results of DNA fragmentation provide information as to the tissue or cell type of origin or origination from a cancerous or non-cancerous cell. Examples of fragmentation analysis include analysis of fragment length, fragment location, distribution of fragment length (i.e., average length), fragmentation-based nucleosome detection, fragment pattern analysis, analysis of fragment end sequences, evaluating effects of fragmentation with specific nucleases and binding of DNA-binding proteins. In some embodiments, the fragmentation analysis is fragment length analysis. In some embodiments, fragment length is average fragment length. In some embodiments, fragment length is the distribution of fragment lengths in a plurality of fragments. In some embodiments, the fragmentation analysis is fragmentation locational analysis. In some embodiments, the fragmentation analysis analyzes the location of the fragments in the genome. In some embodiments, the fragmentation analysis analyzes the location of the fragment point in a sequence. In some embodiments, fragmentation analysis comprises fragment end sequence analysis. In some embodiments, a fragment end sequence is a fragment end motif. In some embodiments, the fragment end is a fragment jagged end. In some embodiments, fragmentation analysis comprises analysis of a fragmentation pattern. In some embodiments, fragmentation analysis comprises analysis of DNA binding protein binding. In some embodiments, fragmentation analysis is fragmentation-based DNA-binding protein binding analysis. In some embodiments, fragment analysis comprises actively fragmenting the DNA. In some embodiments, the DNA binding protein is a transcription factor. In some embodiments, the DNA binding protein is an insulator. In some embodiments, the insulator is CTCF. In some embodiments, the transcription factor is an NKX transcription factor. In some embodiments, the active fragment is with a nuclease. It will be understood by a skilled artisan that fragmentation analysis cannot be properly performed with bisulfite converted DNA. This is because bisulfite conversion changes the sequence of the DNA.

In some embodiments, the identifying is based on the sequence and the copy number analysis. In some embodiments, the identifying is based on the DNA modification data and the copy number analysis. In some embodiments, the identifying is based on the sequence, DNA modification data and copy number analysis. In some embodiments, the identifying is based on the sequence, fragmentation analysis and the copy number analysis. In some embodiments, the identifying is based on the DNA modification data, fragmentation analysis and the copy number analysis. In some embodiments, the identifying is based on the sequence, DNA modification data, fragmentation analysis and copy number analysis. In some embodiments, the copy number analysis is performed with the sequence determined from sequencing a plurality of DNAs. In some embodiments, the presence of an abnormal copy number indicates the DNA is from a cancer cell. In some embodiments, an abnormal copy number is any number other than 2.

In some embodiments, the identifying is based on the sequence and the fragmentation analysis. In some embodiments, the identifying is based on the DNA modification data and the fragmentation analysis. In some embodiments, the identifying is based on the sequence DNA modification data and fragmentation analysis. In some embodiments, the fragment end sequence analysis is performed with the sequence determined from sequencing a plurality of DNAs. In some embodiments, the presence of a specific end fragment sequence indicates the DNA is from a cancer cell. In some embodiments, an enrichment of a specific end fragment sequence indicates the sample is from a subject that has cancer. In some embodiments, the end sequence is an end 4 nucleotides. In some embodiments, the end sequences are the sequences provided in Chan, 2020. In some embodiments, the end sequence is selected from CCCA, CCAG, CCTG, CCAA, CCCT, CCTT, CCAT, CAAA, CCTC. CCAC, TGAA, TAAA, CCTA, CCCC, TGAG, TGTT, CAAG, CTTT, AAAA, TGTG, CATT, CACA, CAGA, TATT, AND CAGG. In some embodiments, the end sequence is CCCA. In some embodiments, the end sequence is CCTG. In some embodiments, the end sequence is AAAA. In some embodiments, the presence of a specific end fragment sequence indicates the DNA is from a specific tissue. In some embodiments, the presence of a specific end fragment sequence indicates the DNA is from a specific cell type.

By another aspect, there is provided a method of producing an adapter ligated cfDNA library, the method comprising:
a. providing a sample comprising cfDNA;
b. ligating an adapter to the cfDNA to produce adapter ligated cfDNA;
c. removing unligated adapter from the adapter ligated cfDNA by a cleanup step comprising a first SPRI bead size exclusion and a second SPRI bead size exclusion;
   thereby producing an adapter ligated cfDNA library.
   In some embodiments, the adapter ligated cfDNA library is for use with a nanopore apparatus. In some embodiments, the adapter ligated cfDNA library is for use in nanopore sequencing. In some embodiments, sequencing is sequencing of the library. In some embodiments, the adapter ligated cfDNA library is for use in a method of the invention. In some embodiments, the adapter ligated cfDNA library is the sample provided for step (a). In some embodiments, the adapter ligated cfDNA library is the sample. In some embodiments, the method further comprises passing the adapter ligated cfDNA library through a nanopore apparatus. In some embodiments, the passing comprises sequencing the cfDNA. In some embodiments, the passing comprises sequencing the library. In some embodiments, the method further comprises using the produced adapter ligated cfDNA library in a method of the invention.
   In some embodiments, the adapter is a short adapter. In some embodiments, the adapter is a very short adapter. In some embodiments, the adapter comprises at most 50 nucleotides. In some embodiments, the adapter comprises at most 61 nucleotides. In some embodiments, the adapter comprises at most 65 nucleotides. In some embodiments, the adapter comprises at most 70 nucleotides. In some embodiments, the adapter comprises at most 75 nucleotides. In some embodiments, the adapter comprises at most 100 nucleotides. In some embodiments, the adapter comprises about 50 nucleotides. In some embodiments, the adapter comprises about 61 nucleotides. In some embodiments, ligating is ligating to the 5′ end. In some embodiments, ligating is ligating to the 3′ end. In some embodiments, ligating is ligating to bot the 5′ and 3′ end. In some embodiments, an end is an end of a cfDNA. In some embodiments, the library is enriched with cfDNA molecules of a size below 200. In some embodiments, the library is enriched with cfDNA molecules of a size between 50 and 200. In some embodiments, the library is enriched with cfDNA molecules of a size between 100 and 200. In some embodiments, the library is enriched with small cfDNA molecules. In some embodiments, the sample is enriched with cfDNA molecules of a size below 200. In some embodiments, the sample is enriched with cfDNA molecules of a size between 50 and 200. In some embodiments, the sample is enriched with cfDNA molecules of a size between 100 and 200. In some embodiments, the sample is enriched with small cfDNA molecules. In some embodiments, the sample is depleted of very small DNA molecules.

In some embodiments, the first SPRI bead size exclusion comprises an SPRI bead to sample ratio of about 0.5:1. In some embodiments, the first SPRI bead size exclusion comprises an SPRI bead to sample ratio of between 0.4:1 and 0.6:1. In some embodiments, the first SPRI bead size exclusion comprises an SPRI bead to sample ratio of 0.5:1 or more. It will be understood that higher concentrations result in greater retention of small DNA. Thus, the first binding can be done even in 1.6×SPRI because the second step will successfully remove the unligated adapter. In some embodiments, the second SPRI bead size exclusion comprises an SPRI bead to sample ratio of about 1.2:1. In some embodiments, the second SPRI bead size exclusion comprises a higher ratio of bead to sample than the first SPRI bead size exclusion. In some embodiments, higher is at least double. In some embodiments, about 1.2:1 is 1.1:1 to 1.3 to 1. In some embodiments, about 1.2:1 is 1:1 to 1.4 to 1. In some embodiments, the second SPRI bead size exclusion comprises an SPRI bead to sample ratio of at least 1.2:1. In some embodiments, the first SPRI bead size exclusion is performed before the second SPRI bead size exclusion.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an.” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”. John Wiley & Sons. New York (1988); Watson et al., “Recombinant DNA”, Scientific American Books. New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds). “Basic and Clinical Immunology” (8th Edition), Appleton & Lange, Norwalk, CT (1994); Mishell and Shiigi (eds). “Strategies for Protein Purification and Characterization-A Laboratory Course Manual” CSHL Press (1996); all of which are incorporated by reference. Other general references are provided throughout this document.

### Materials and Methods

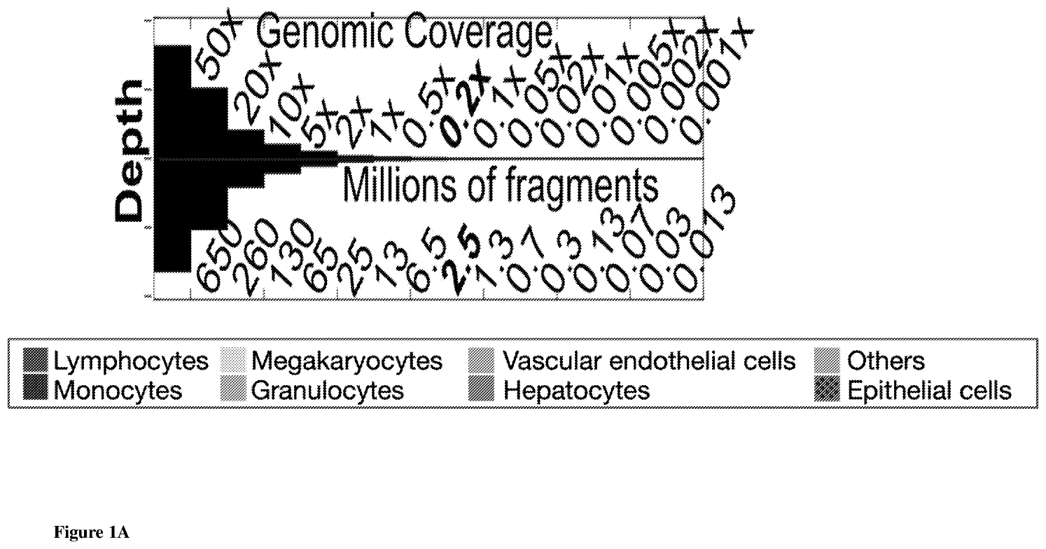

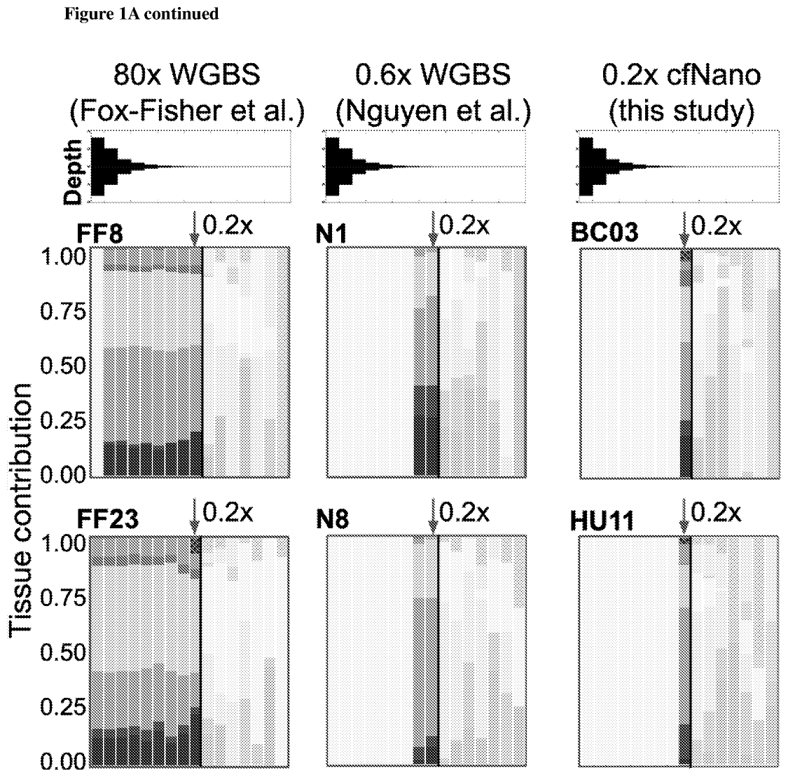

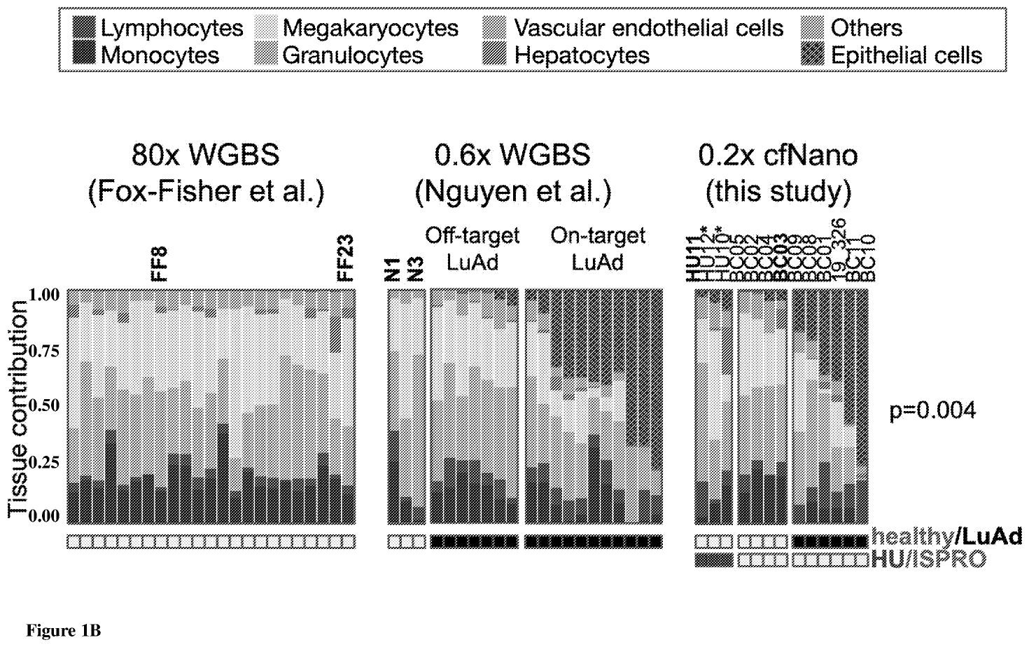

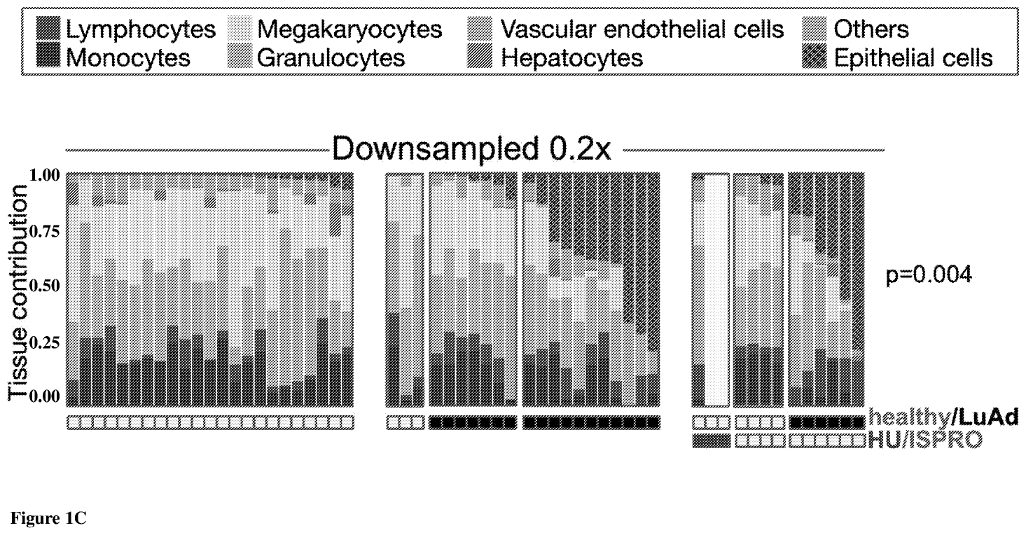

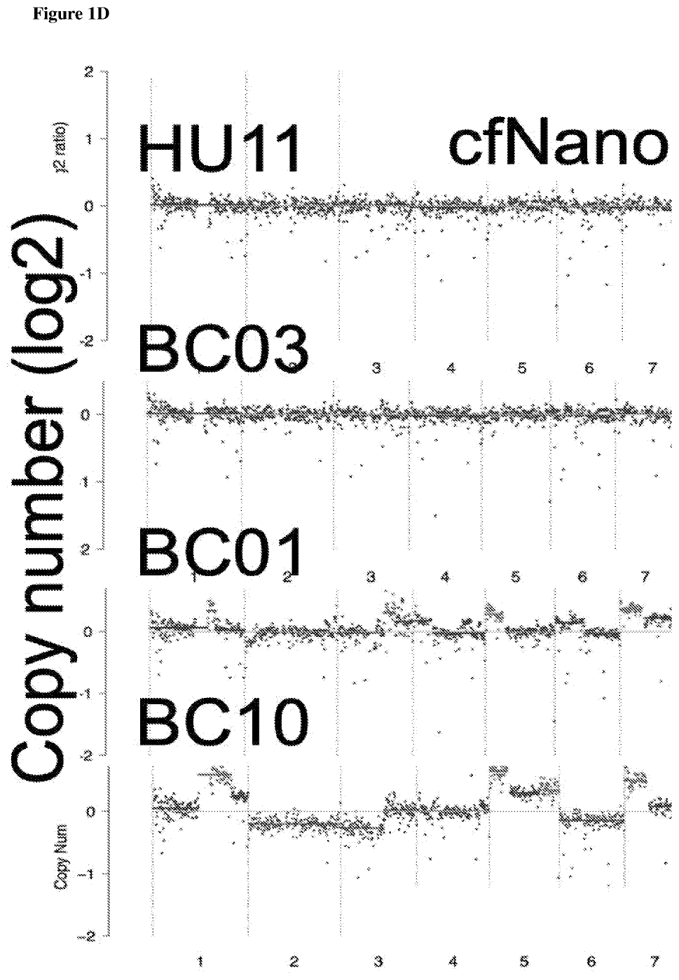

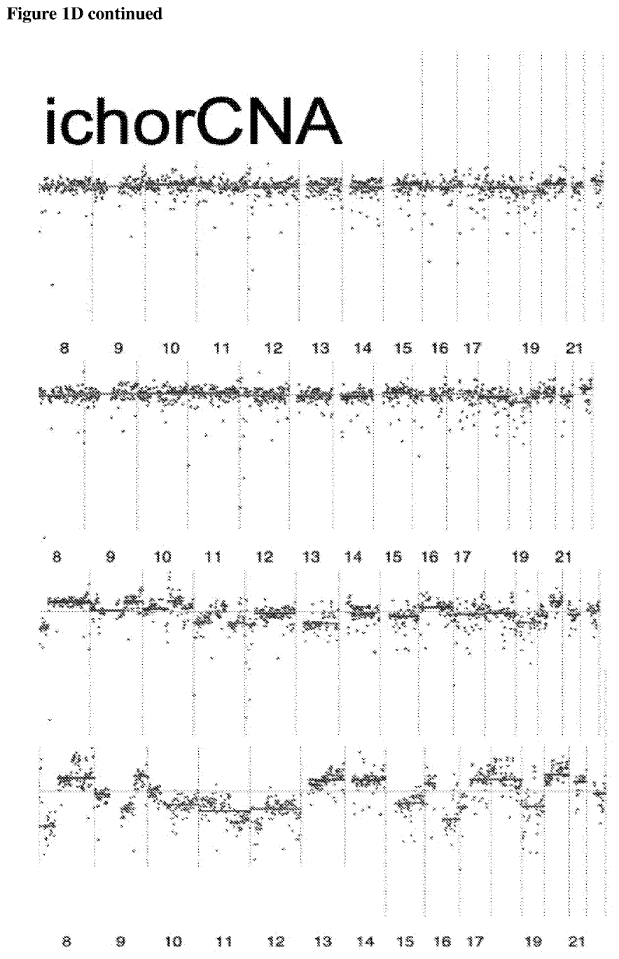

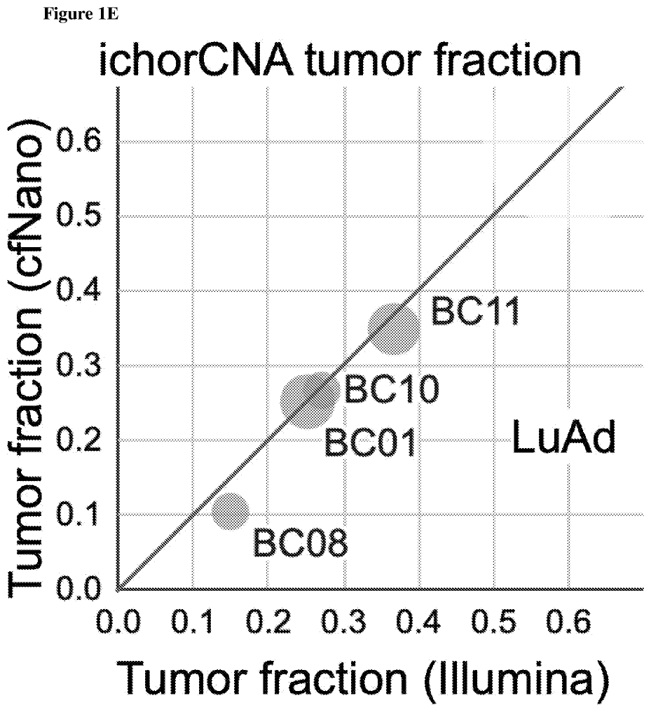

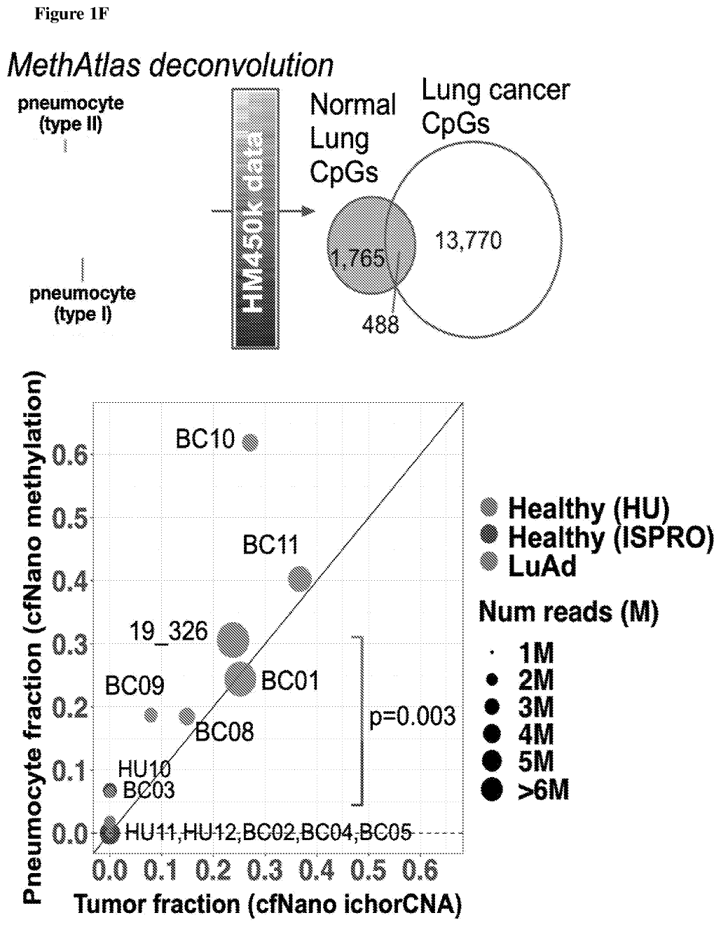

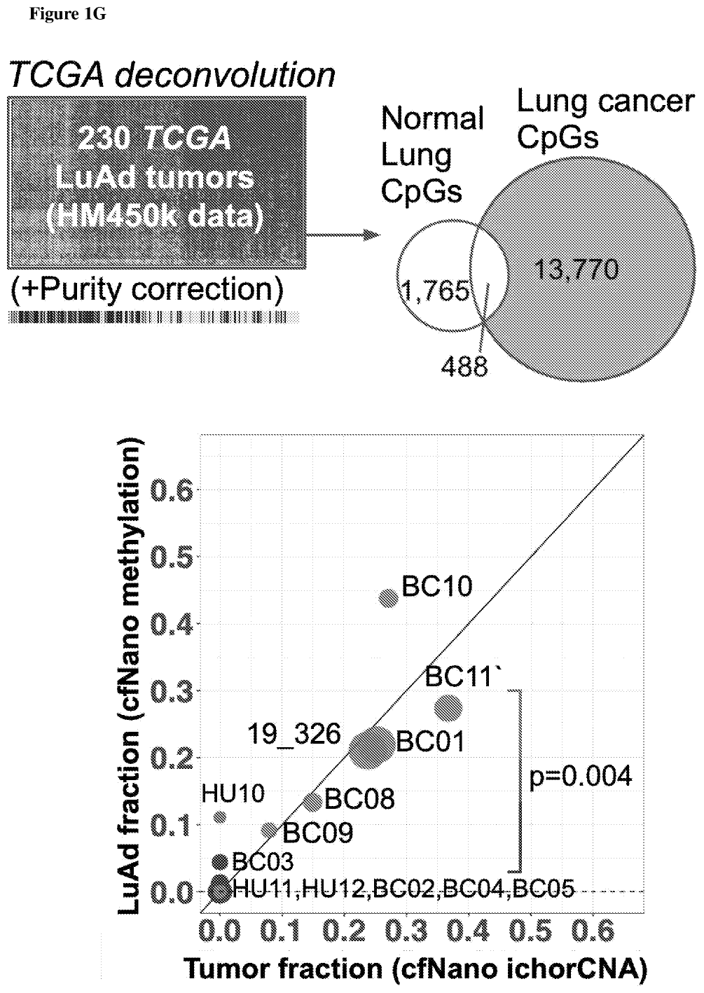

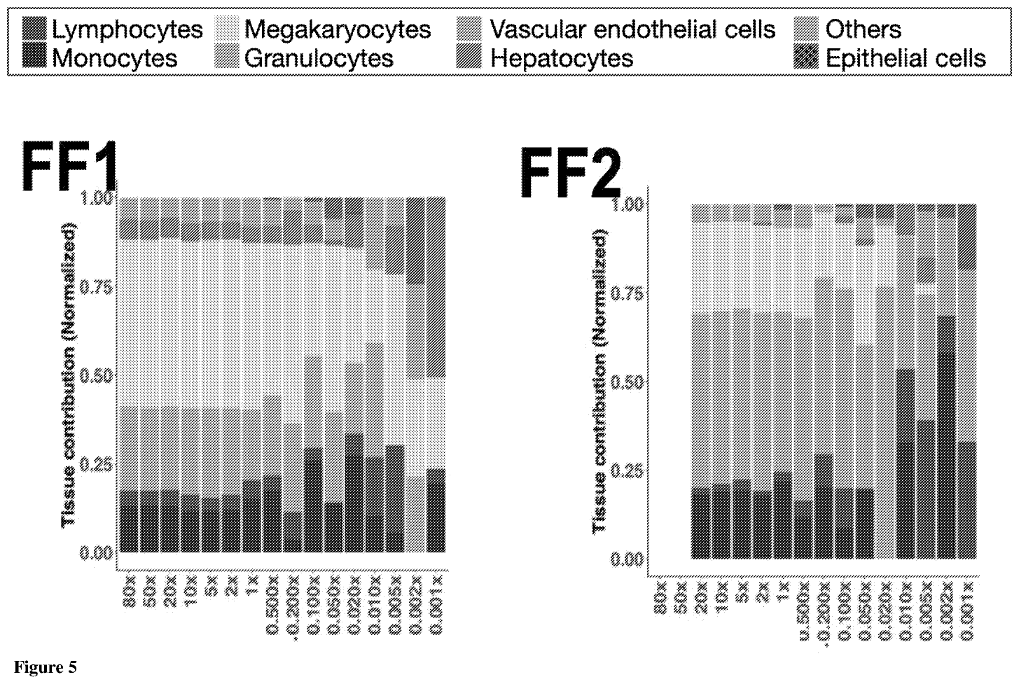

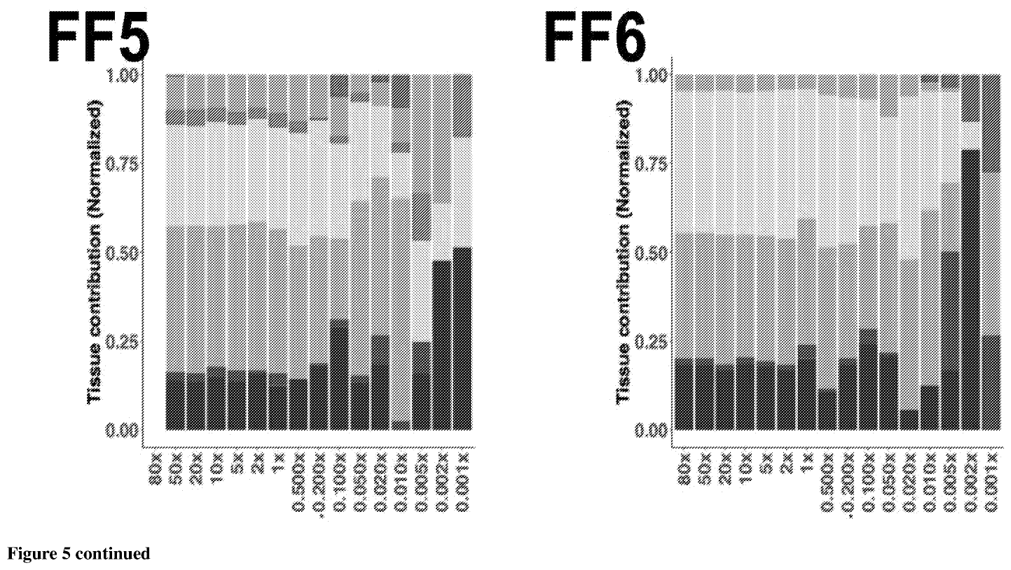

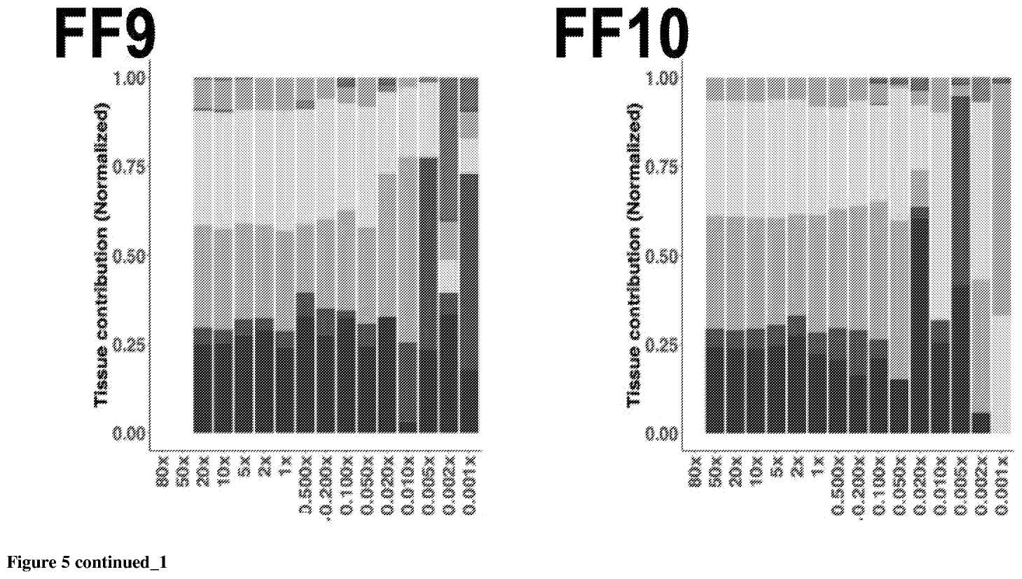

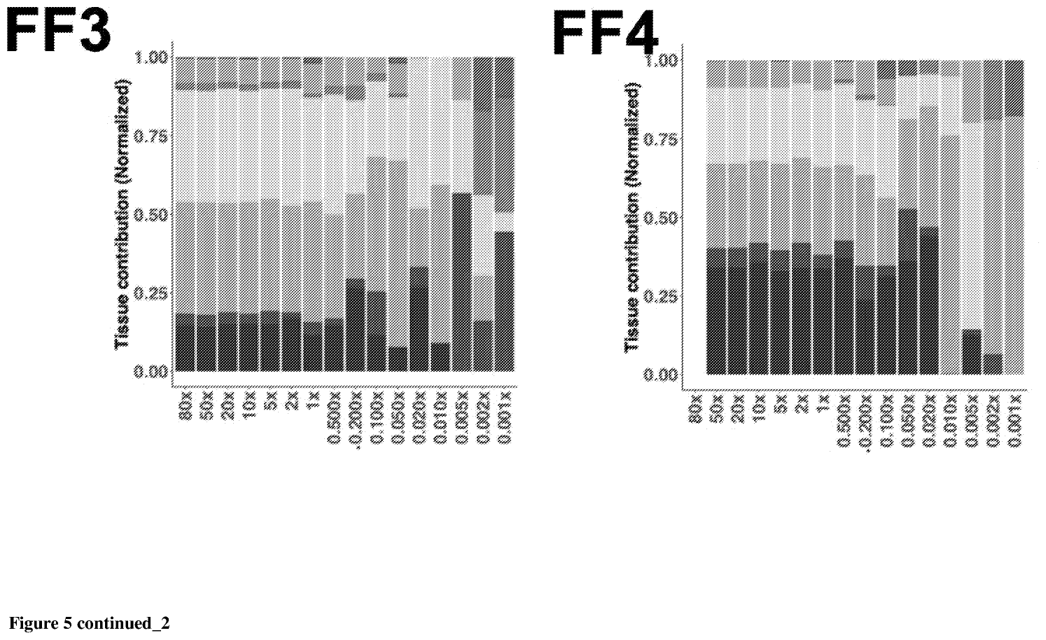

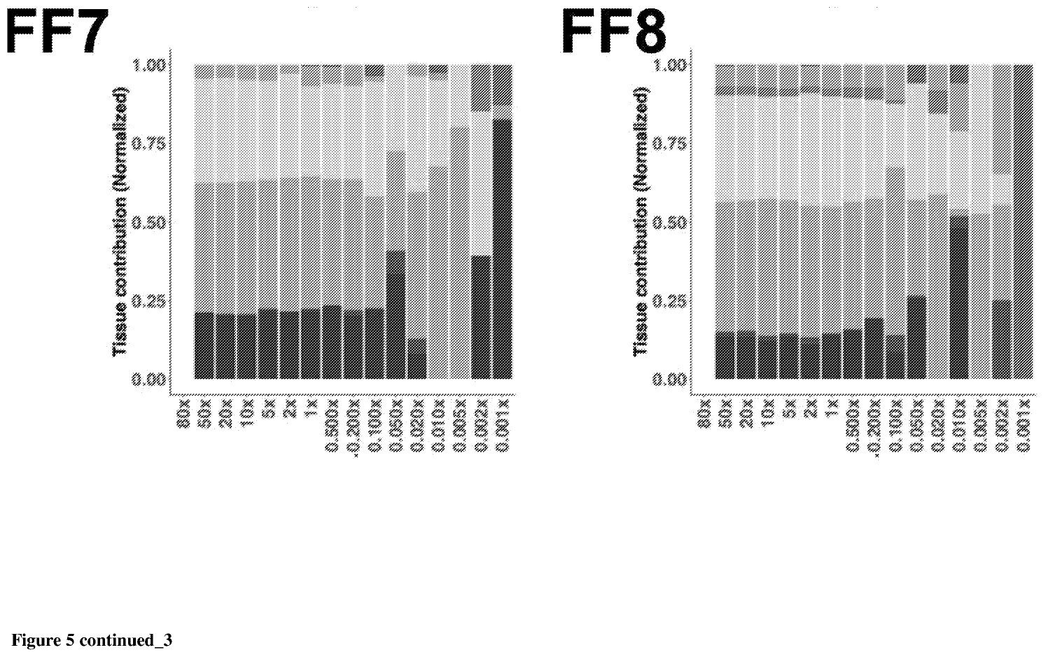

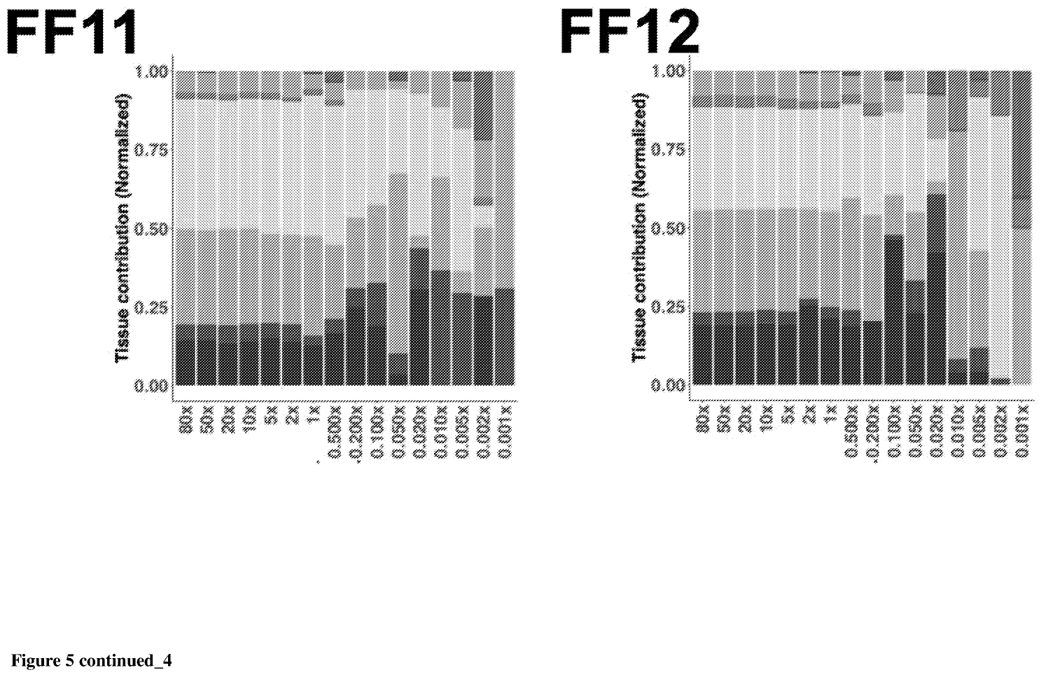

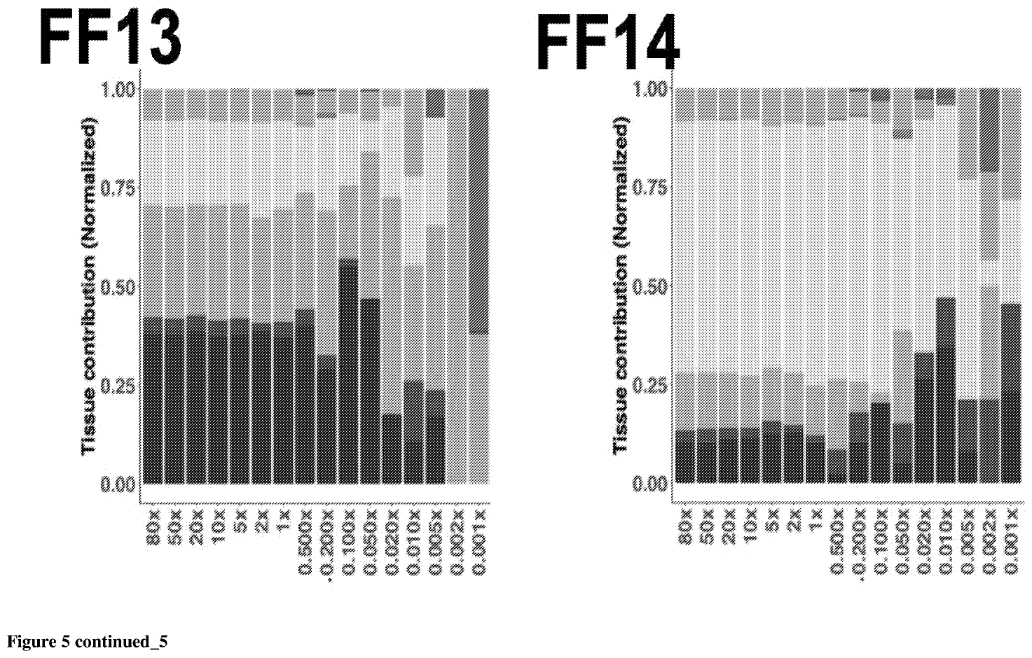

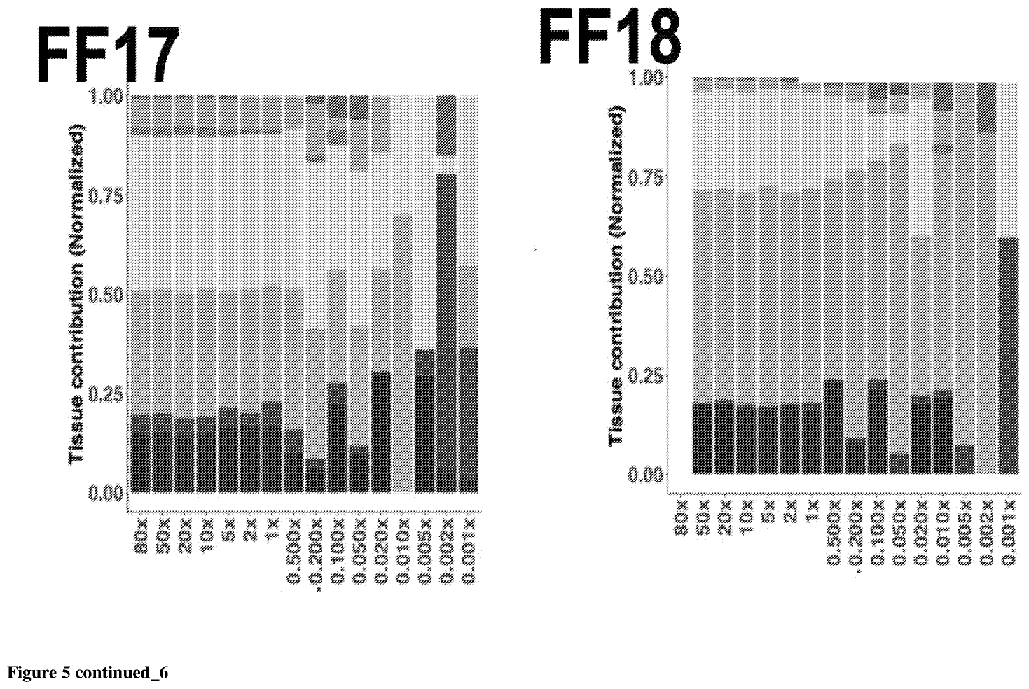

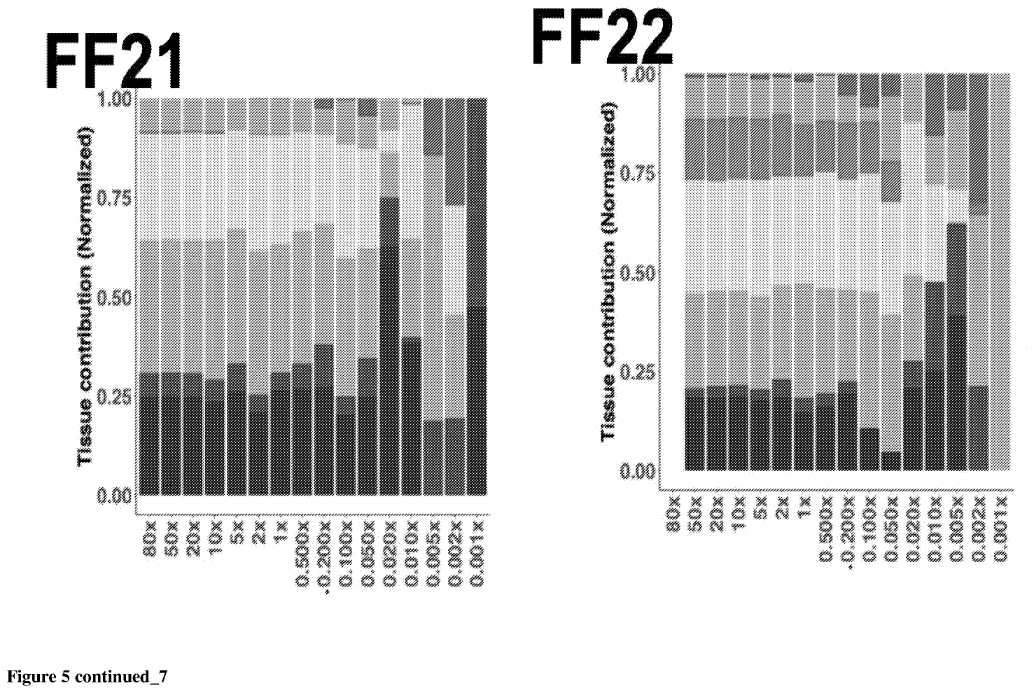

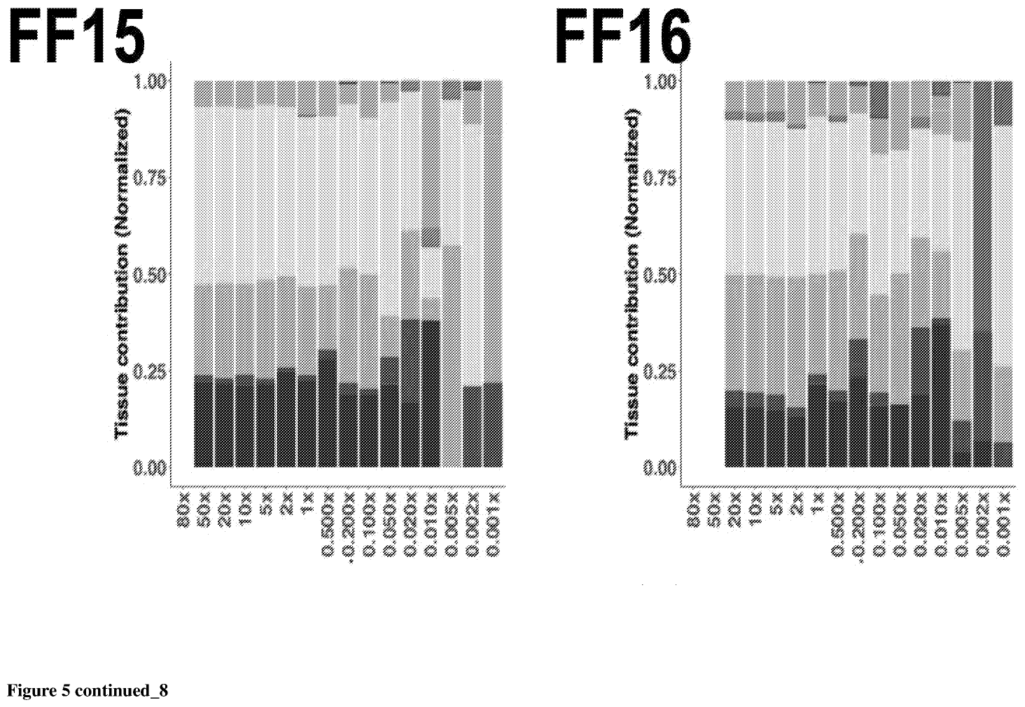

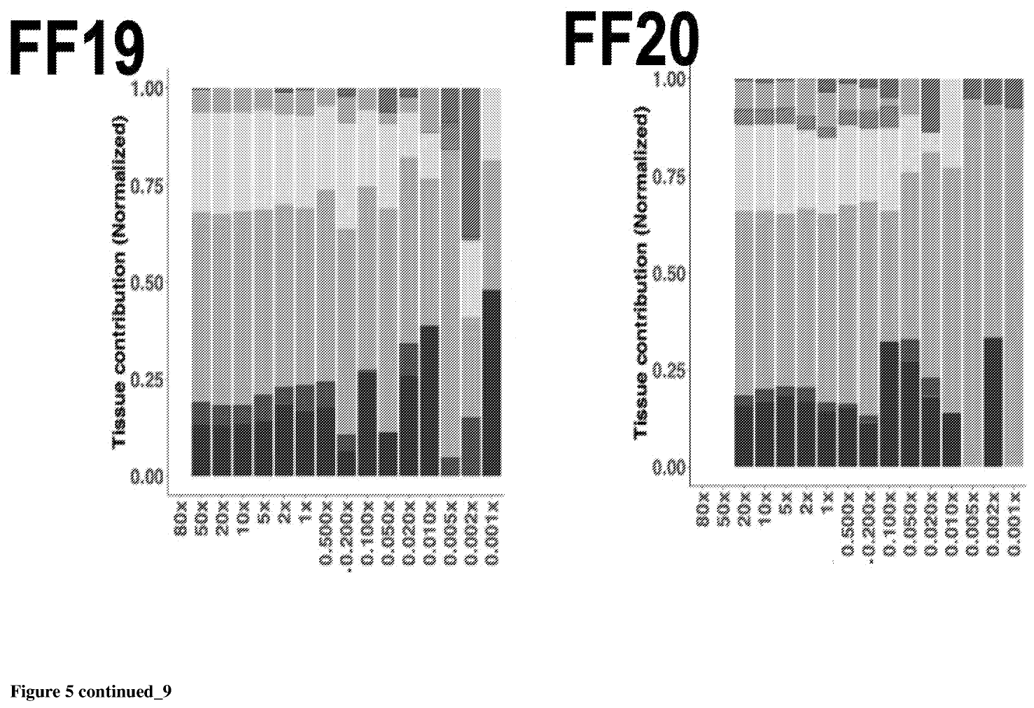

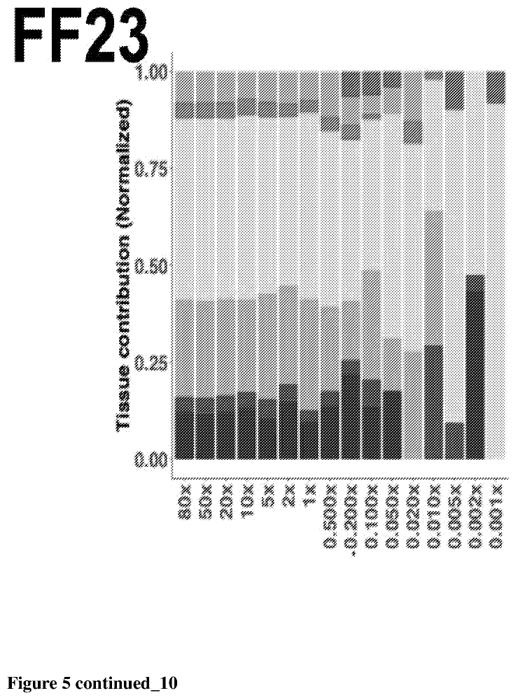

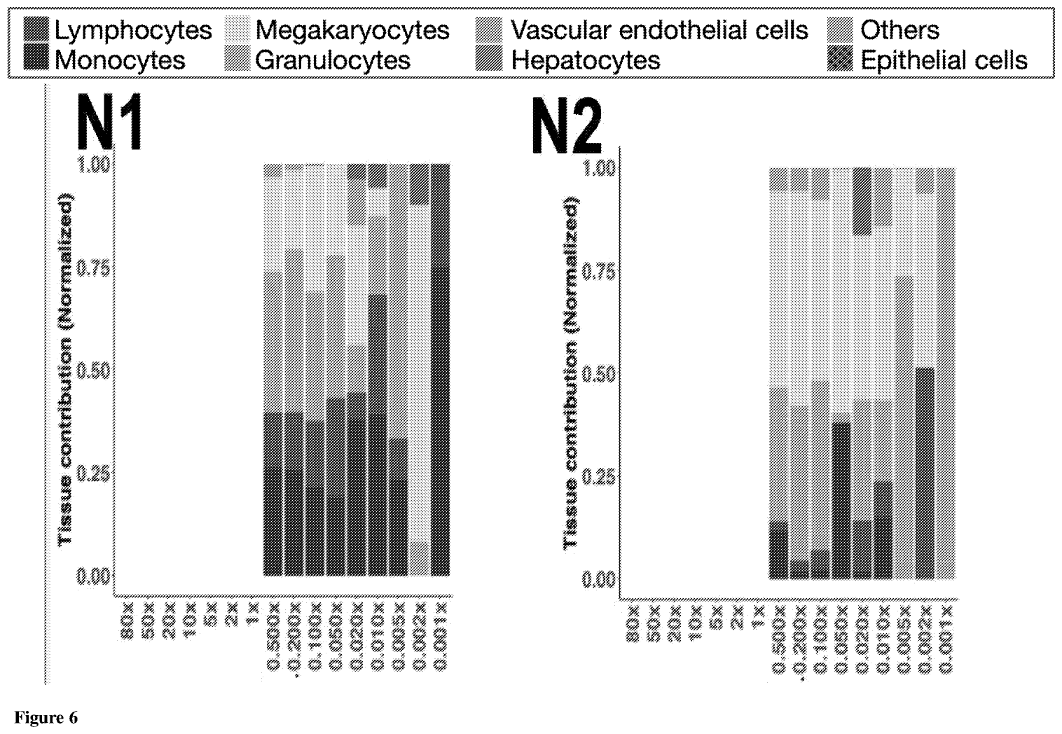

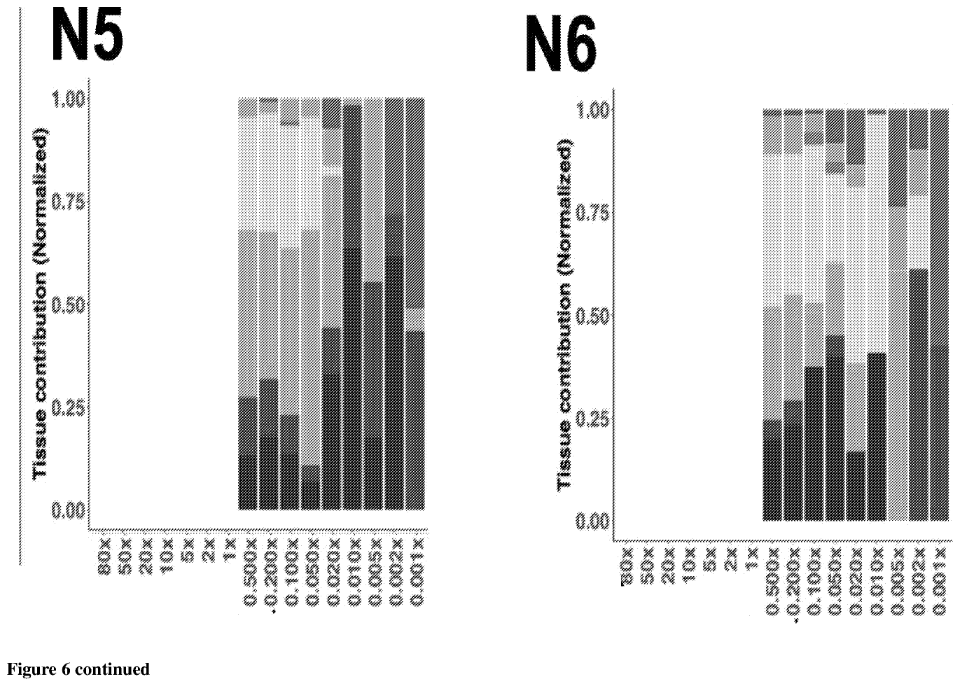

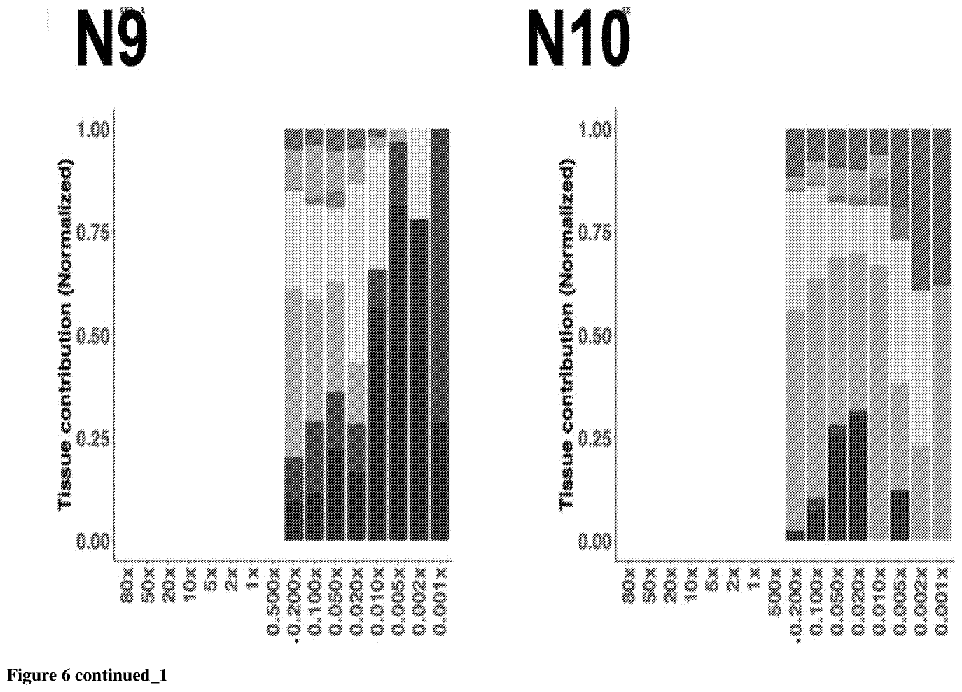

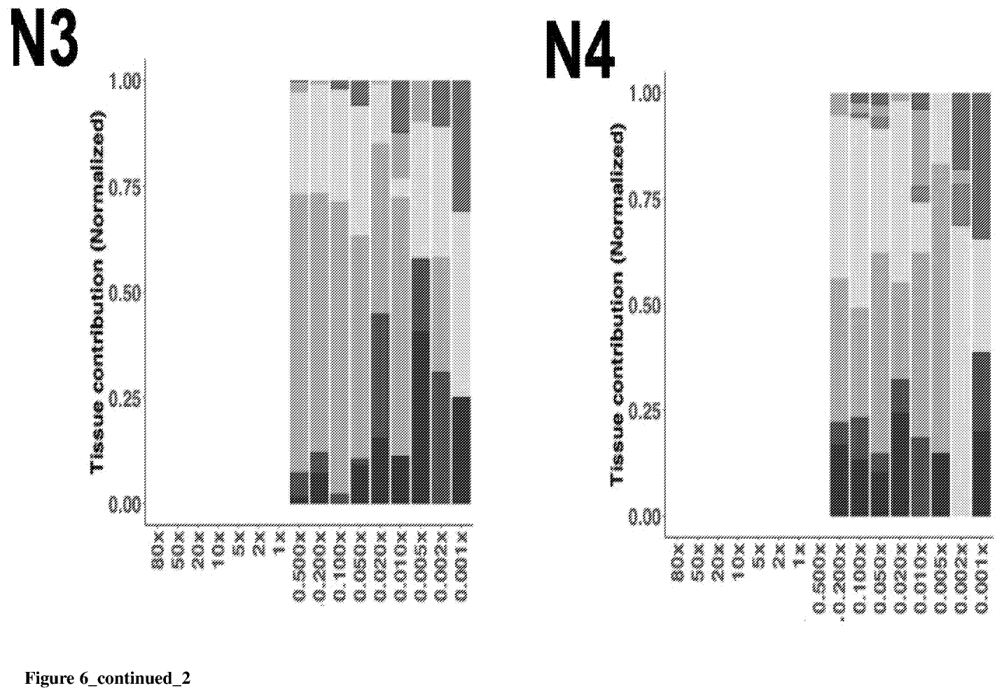

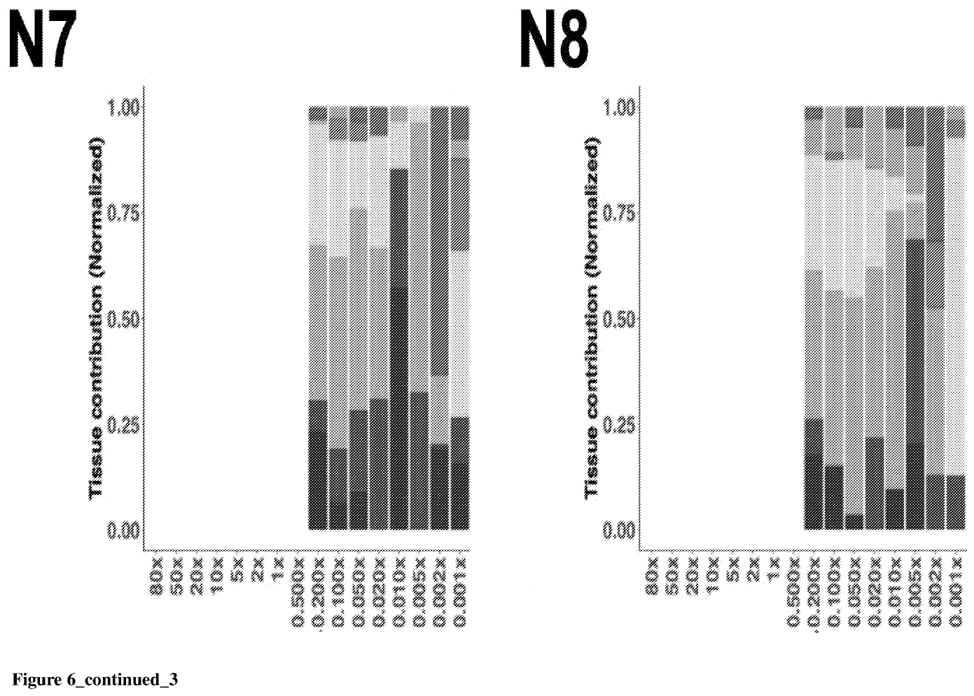

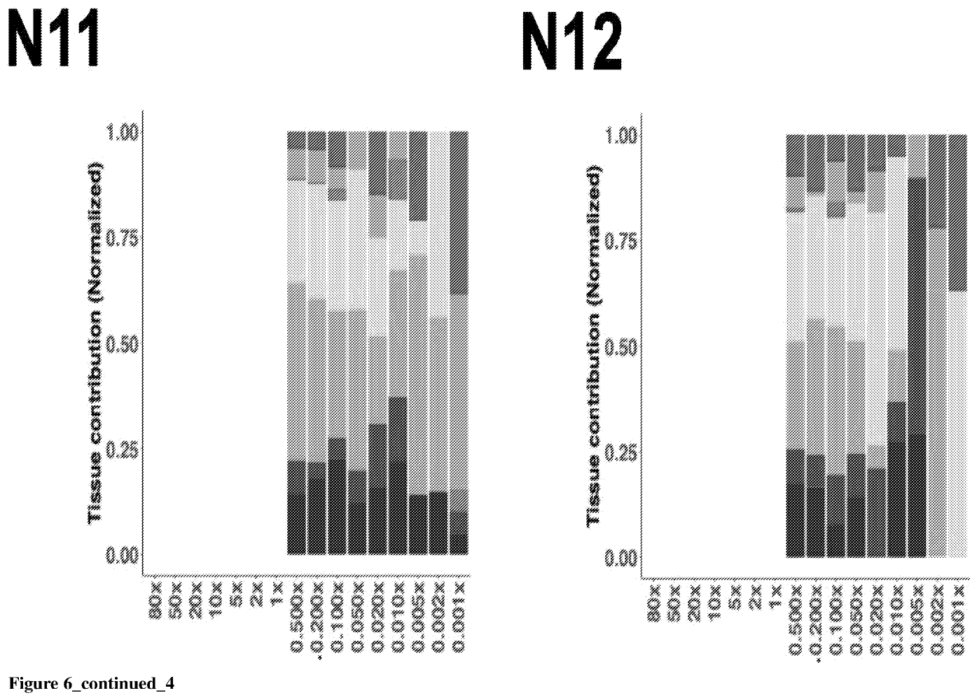

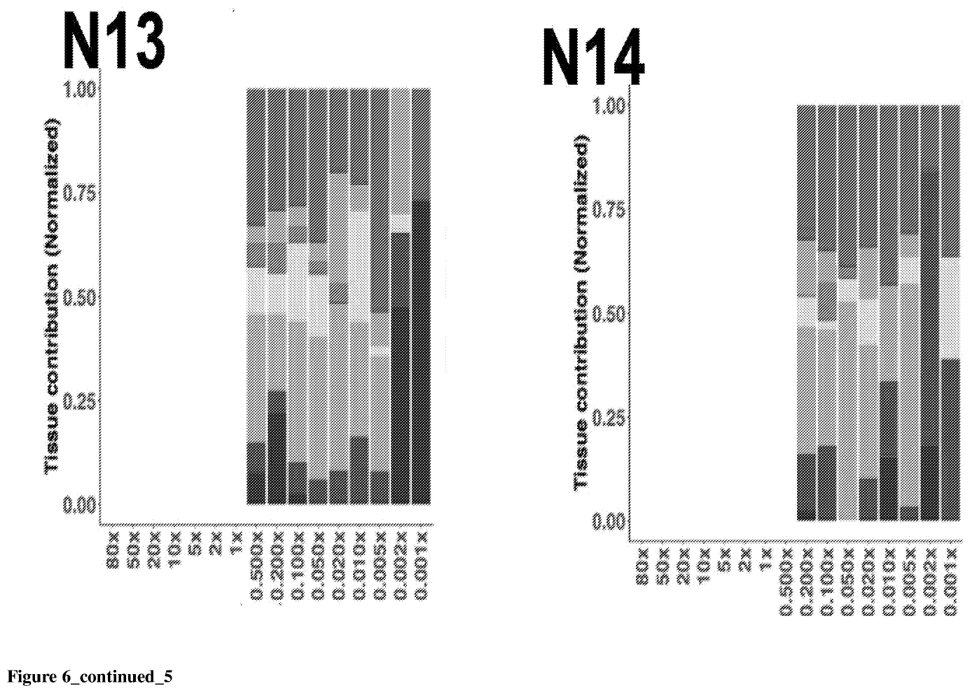

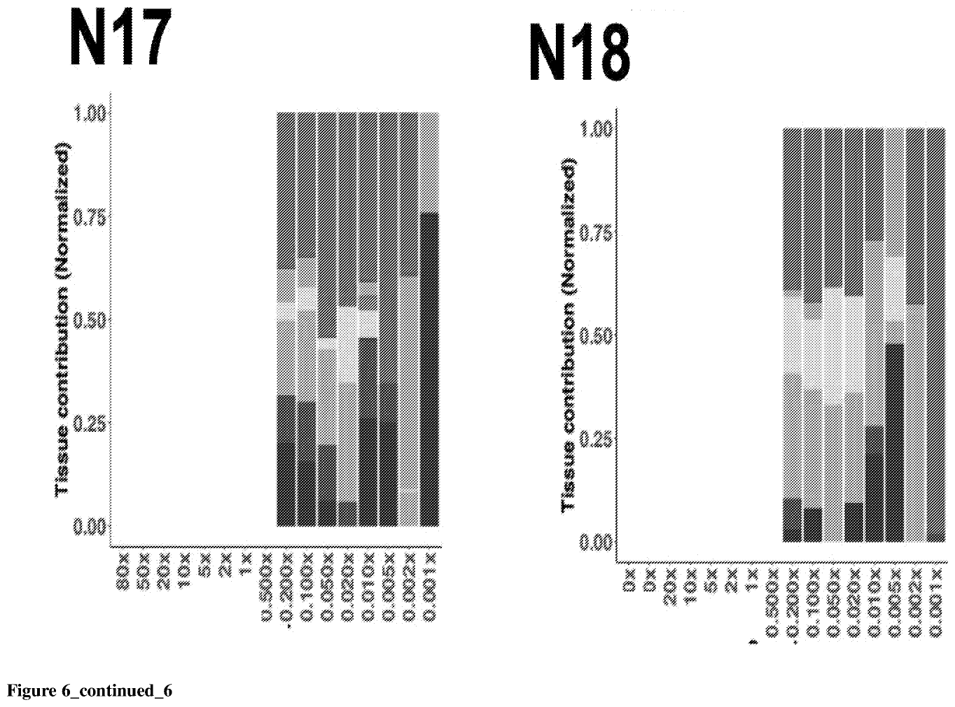

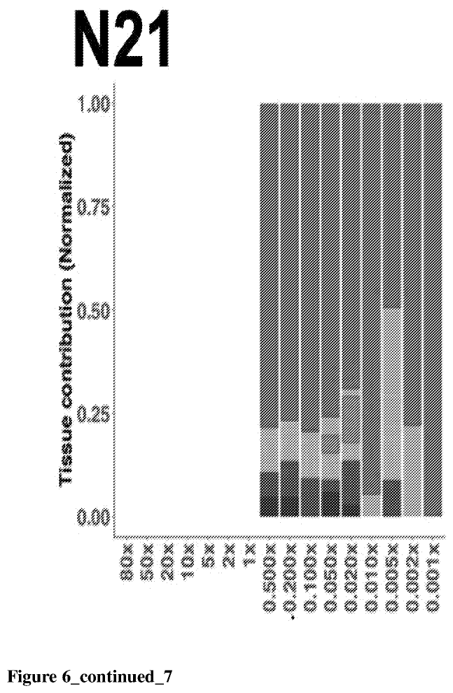

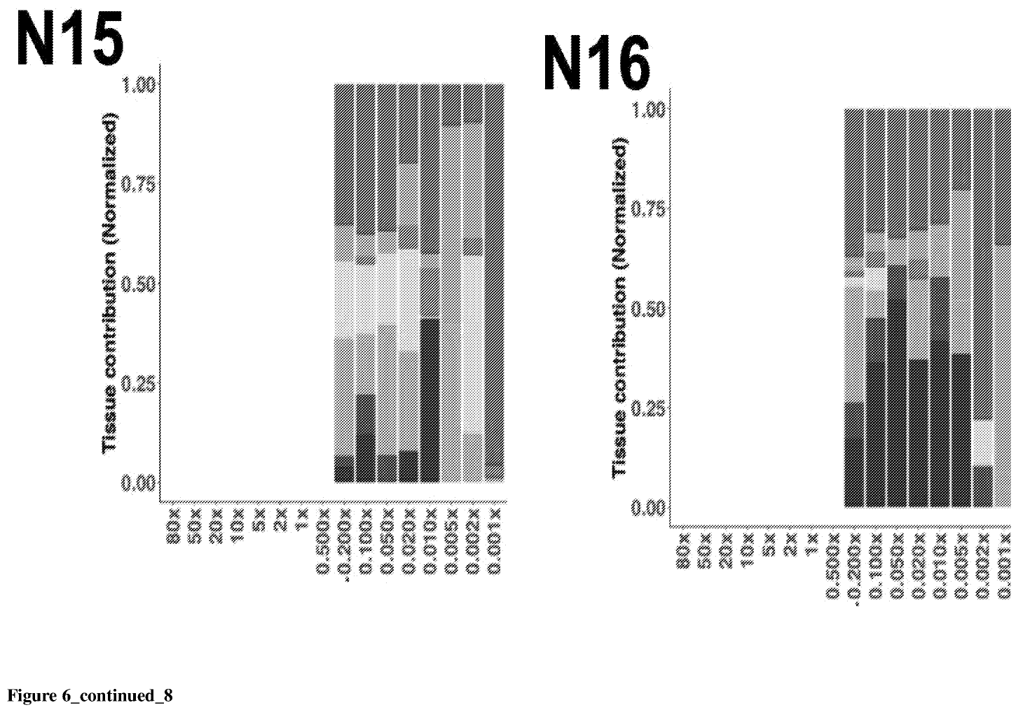

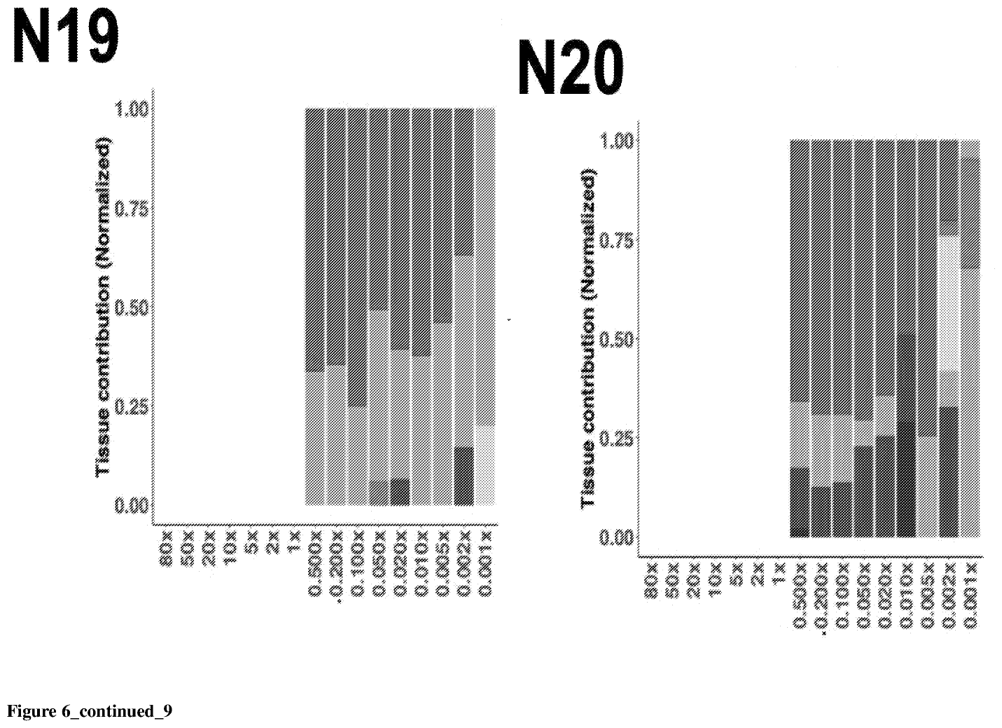

ISPRO Plasma cfDNA samples, library construction, and sequencing. For ISPRO samples in FIGS. 1-14, library construction and sequencing comprised a modified version of the method described previously in Filippo Martignano et al., “Nanopore Sequencing from Liquid Biopsy: Analysis of Copy Number Variations from Cell-Free DNA of Lung Cancer Patients”, Molecular Cancer 20, no. 1 (2021), hereby incorporated by reference in its entirety. Briefly. Blood samples were centrifuged at 1600 g×10″, and plasma was carefully collected with a pipet without disturbing sedimented blood cells. cfDNA was extracted from 4 ml of plasma using QIAamp Circulating Nucleic Acid Kit (QIAGEN, 55114). Library preparation was performed using kits NBD-EXP104 and SQK-LSK109 in order to obtain barcoded libraries. In contrast to the suggested protocol, the bead/sample ratio of SPRI beads (AMPure XP beads, Beckman Coulter, A63880) was increased to 1.8× (from 1× of FFPE/end repair step, 1× of barcode ligation step and 0.5× of adapter ligation step) in all clean-up steps to make sure none of the smaller cfDNA fragments are lost. A higher ratio of SPRI beads was not advisable as it greatly increased the contamination by unligated adapter. An additional adapter cleanup/removal protocol was also tested. In this protocol the 0.5× SPRI separation was followed by a second 1.2×SPRI cleanup.

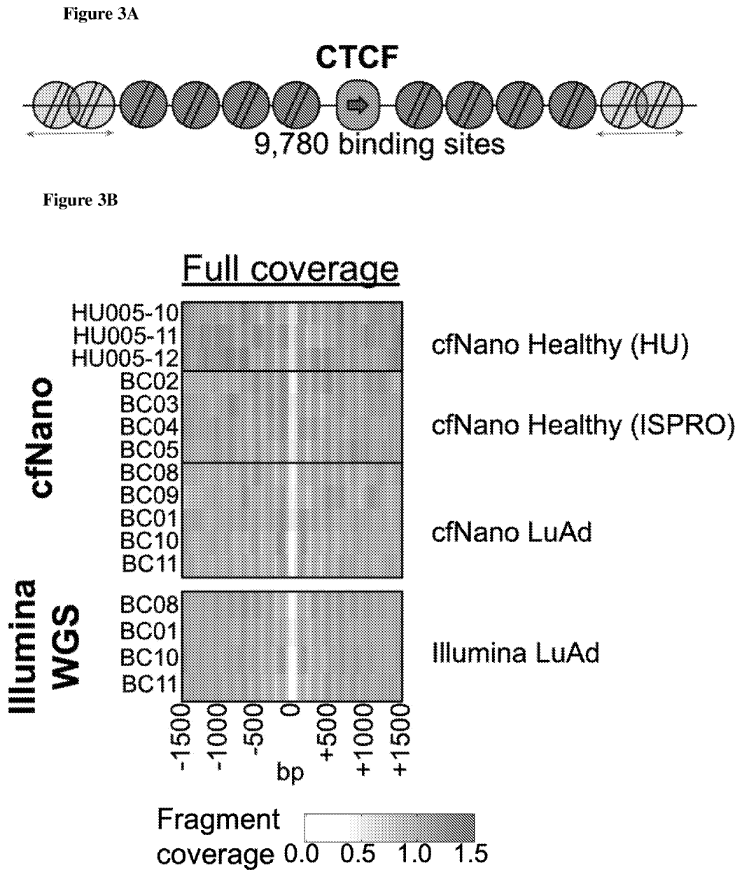

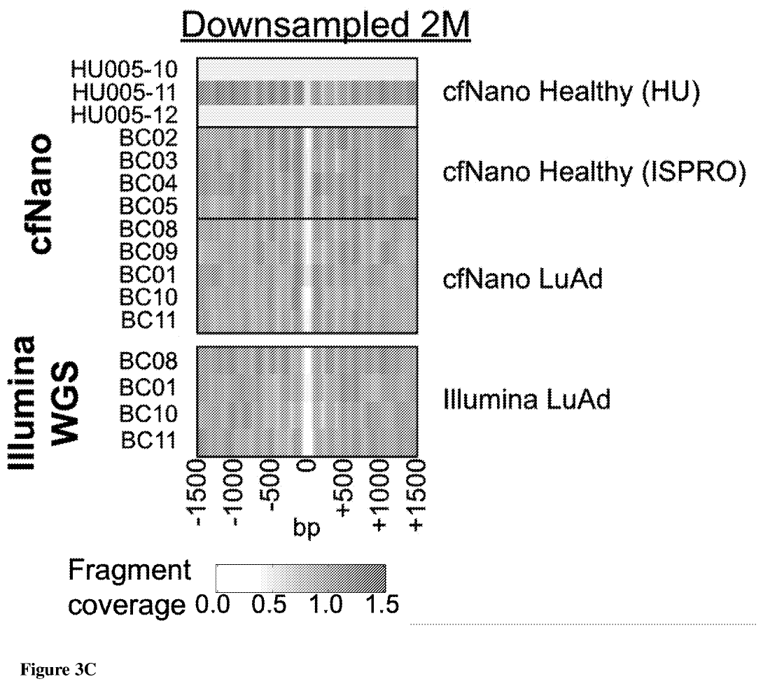

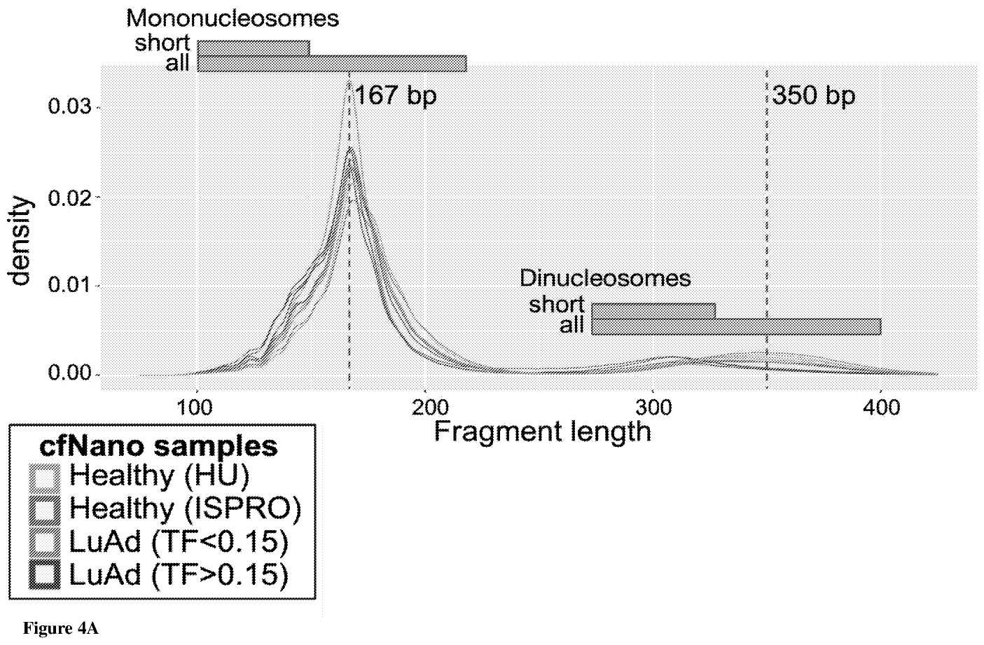

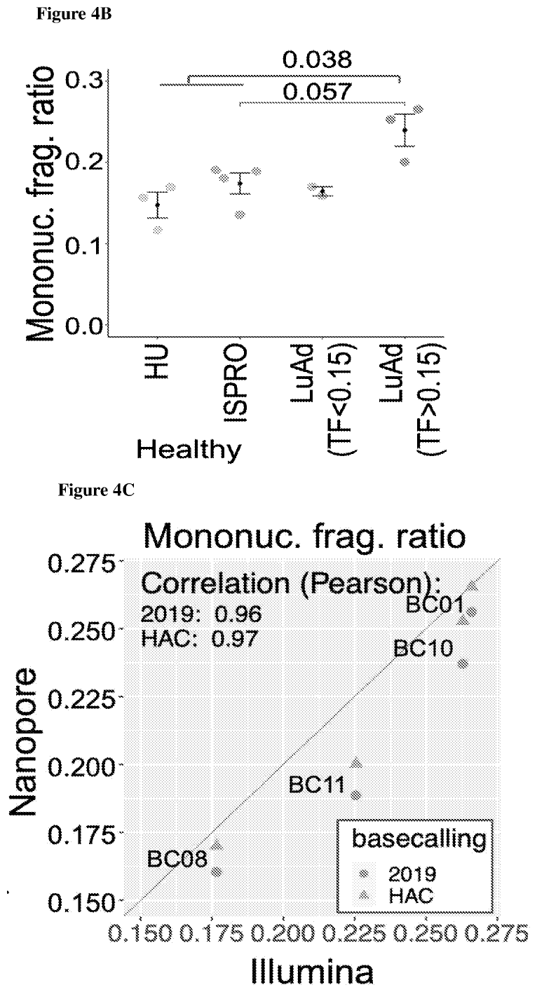

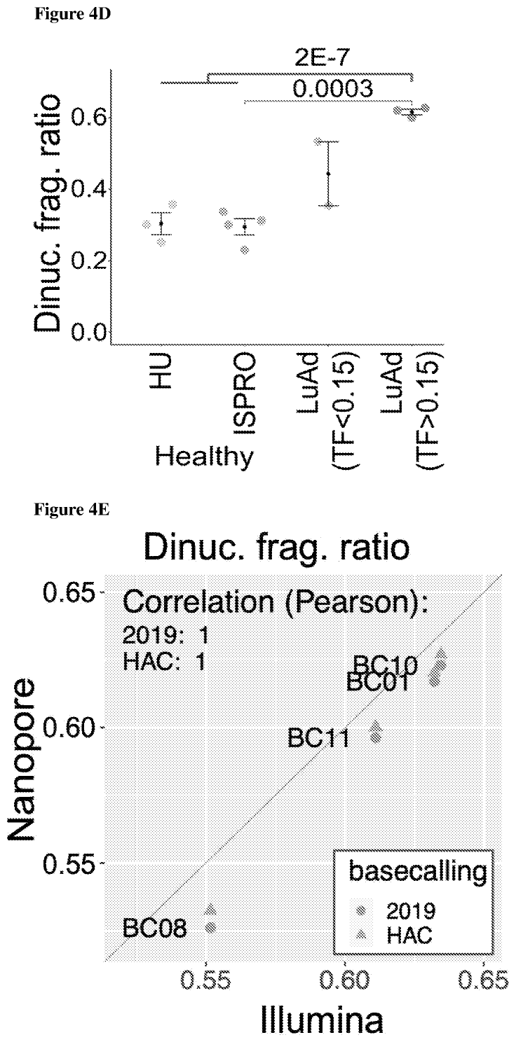

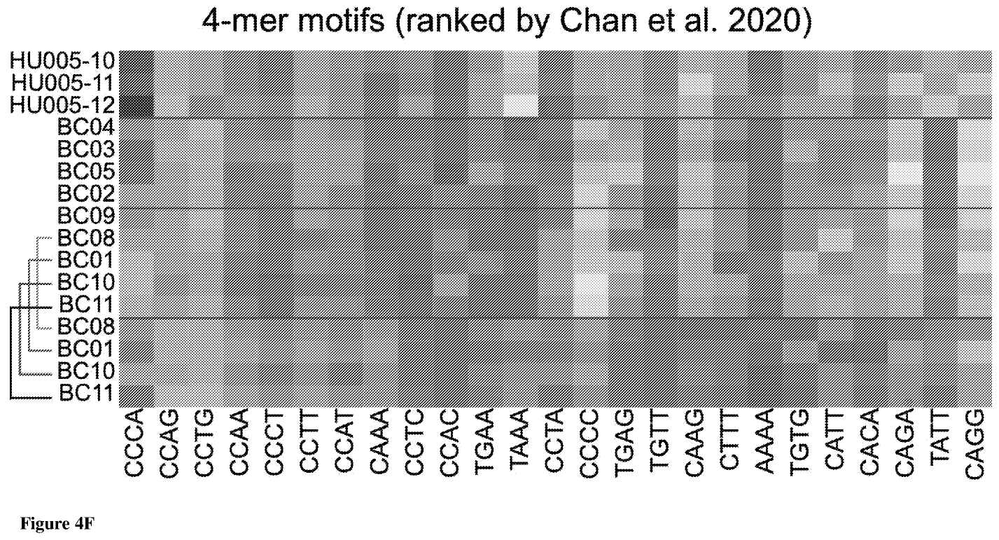

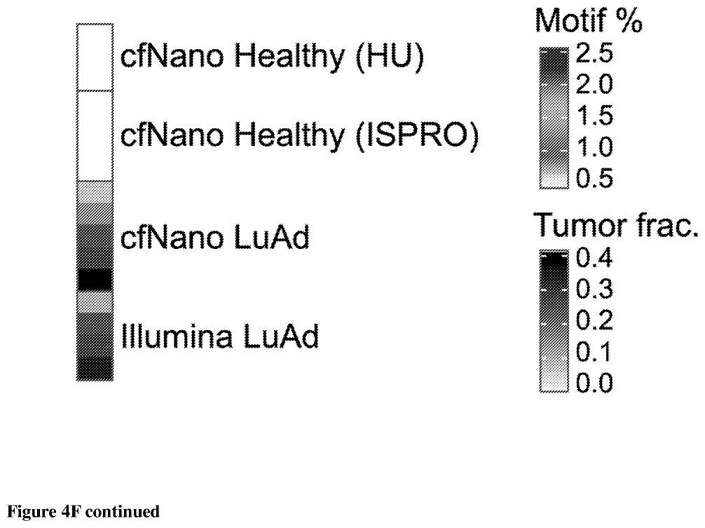

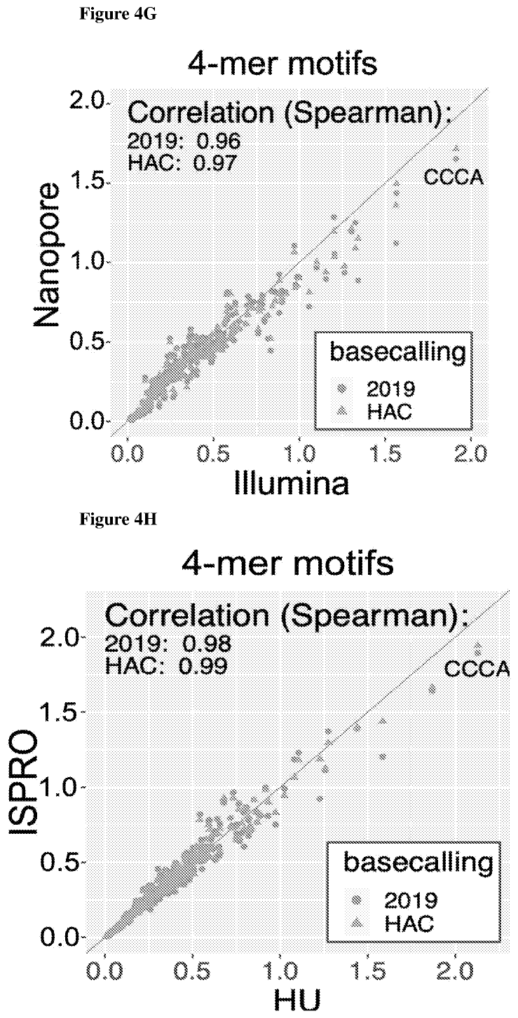

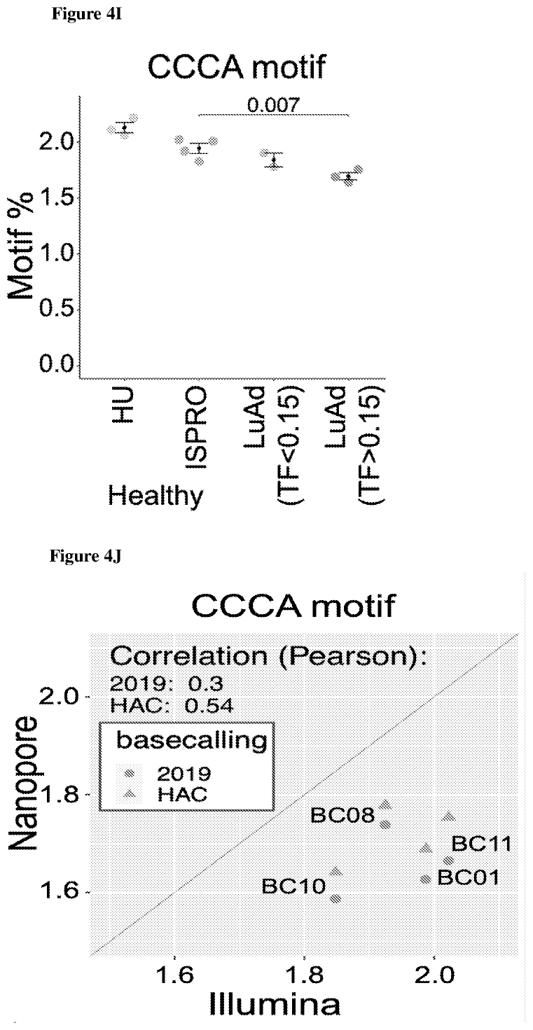

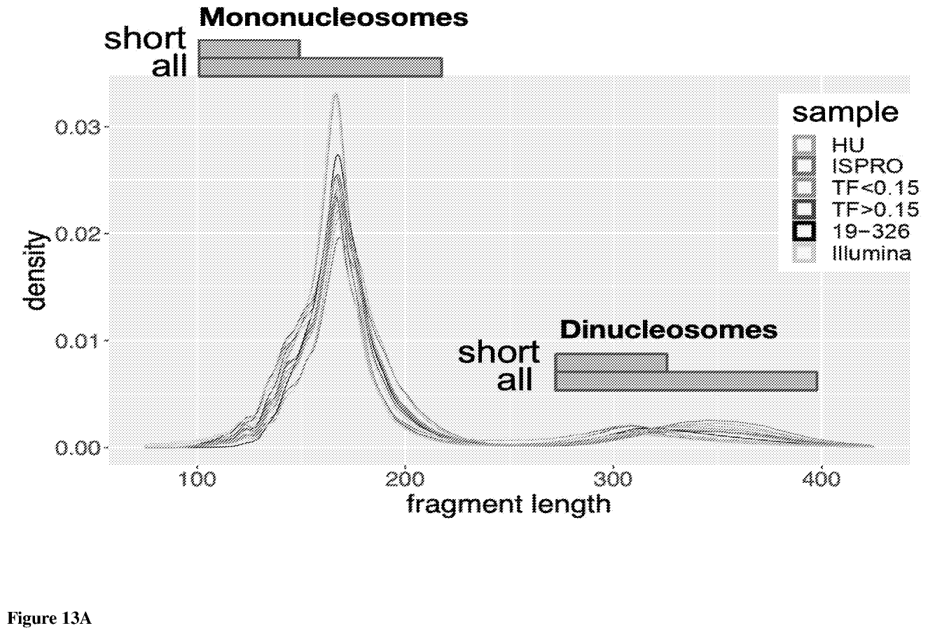

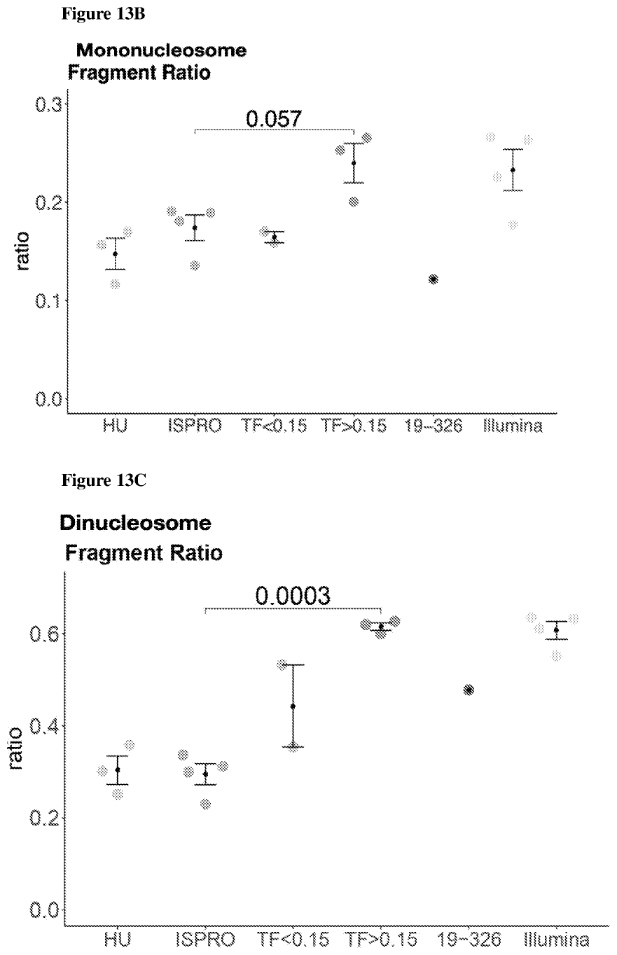

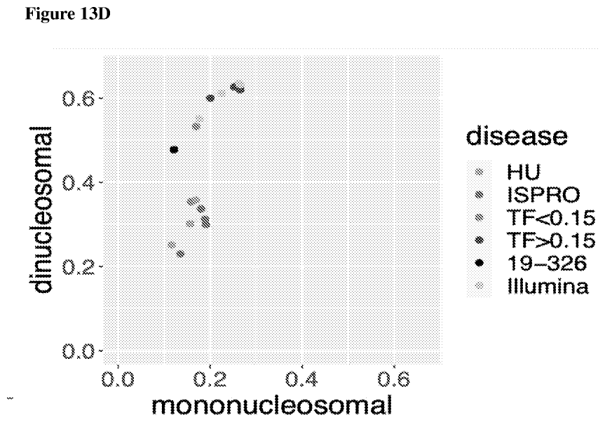

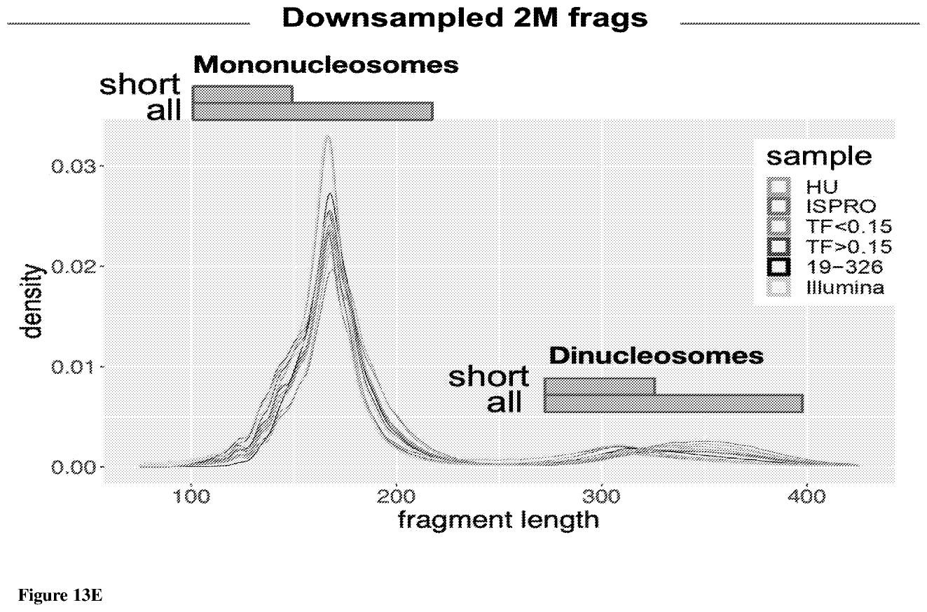

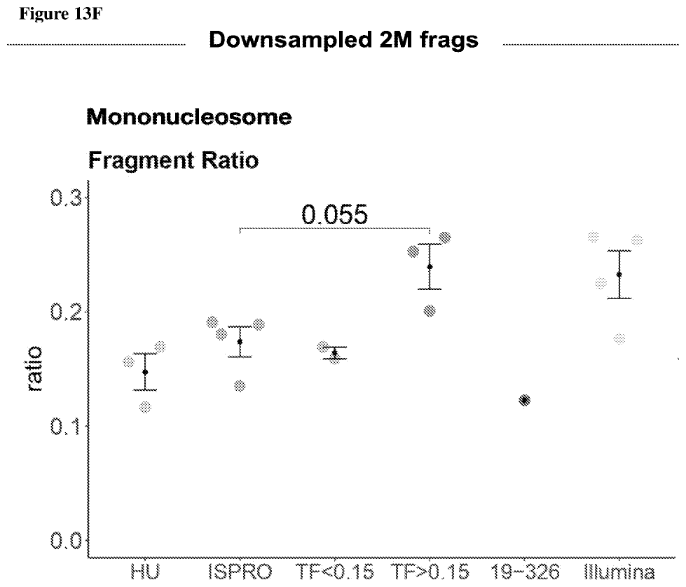

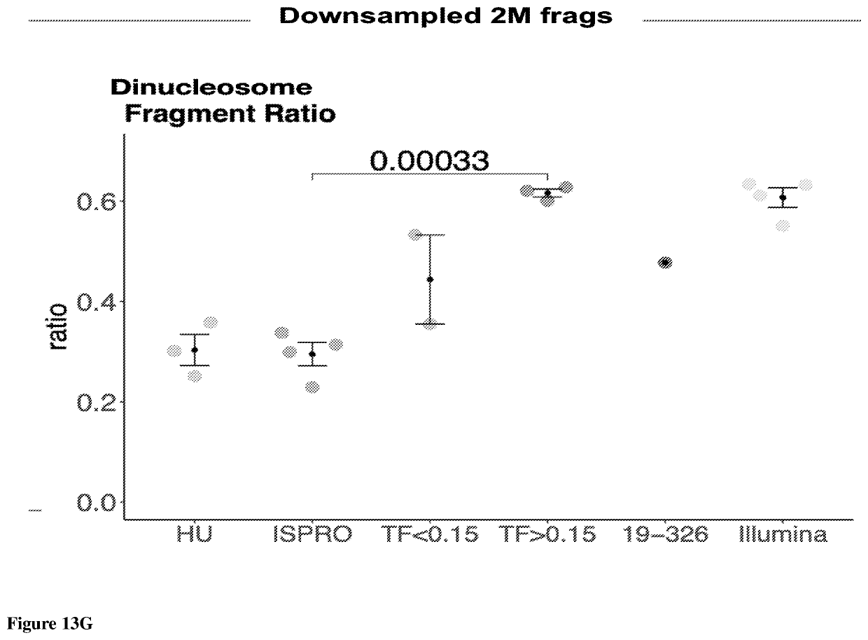

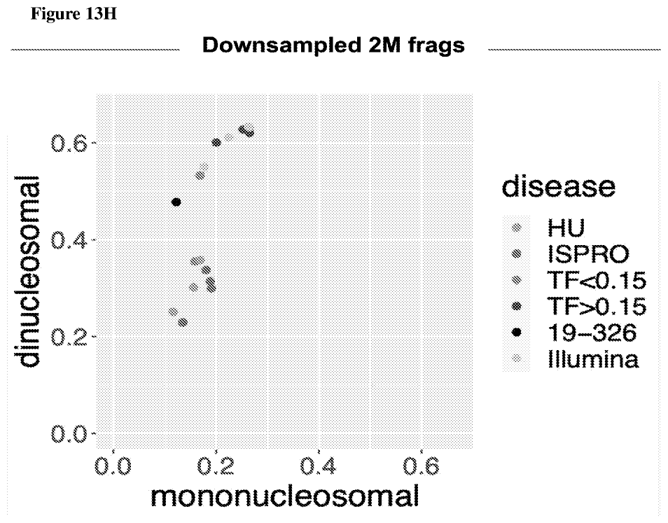

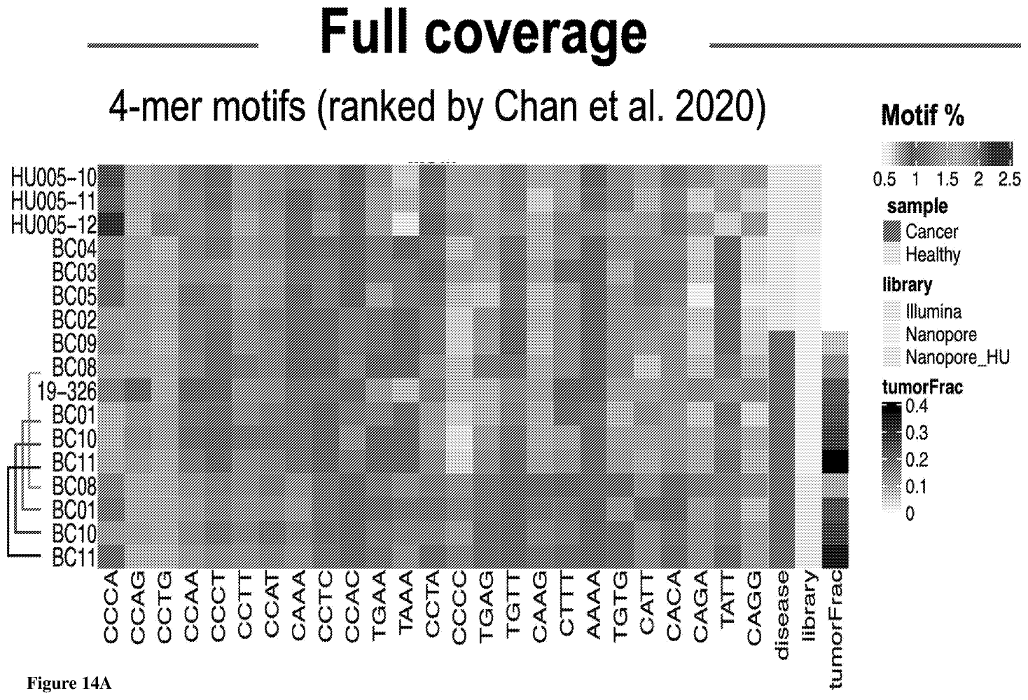

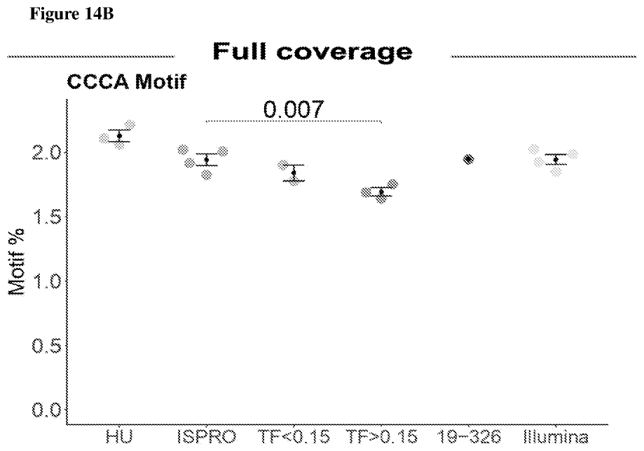

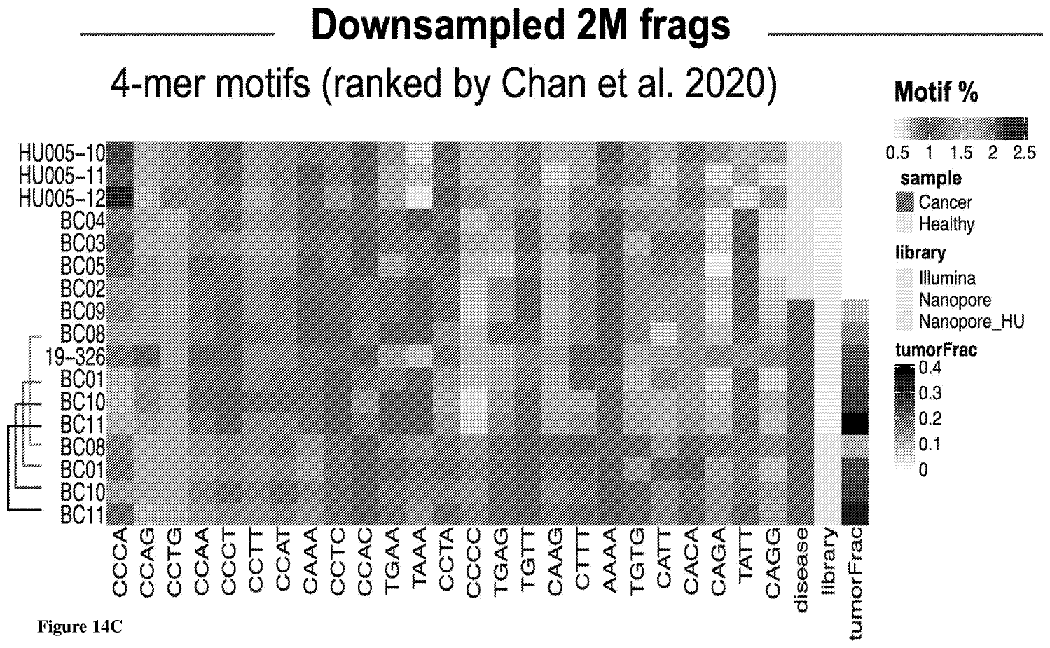

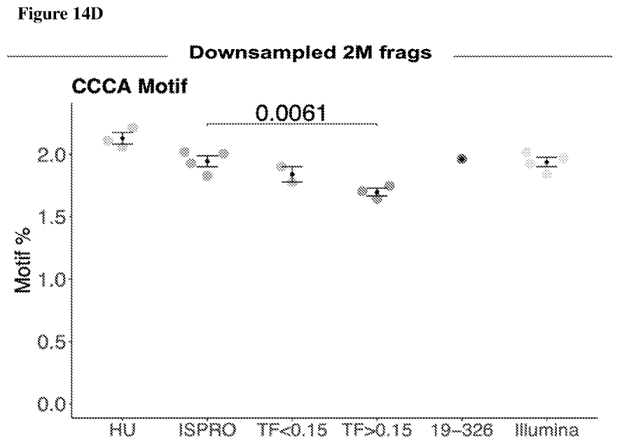

Notably, one sample (S1/19_326) was produced using a different library kit (SQK-LSK109 vs. NBD-EXP104+SQK-LSK109 for all other samples). This is the singleplex library kit, which results in shorter adapter-ligated templates overall (due to the lack of barcodes) and thus responds differently to the equivalent clean up bead concentration and sequencing software settings. Also, adapter trimming is performed differently in 19_326 due to the library kit differences. For these reasons, fragmentomic properties are not directly comparable between 19_326 and other samples. We thus omitted sample 19_326 for the primary fragmentomic analyses (FIG. 3-4) but included it in all primary figures when analyzing methylation and copy number alterations, where small differences in fragment length are not expected to make a difference. We include separate fragmentomic figures that contain sample 19_326 (FIG. 13-14). Standard MinKNOW runtime control was used without modification (S1 using distribution version 18, and all others using version 19).

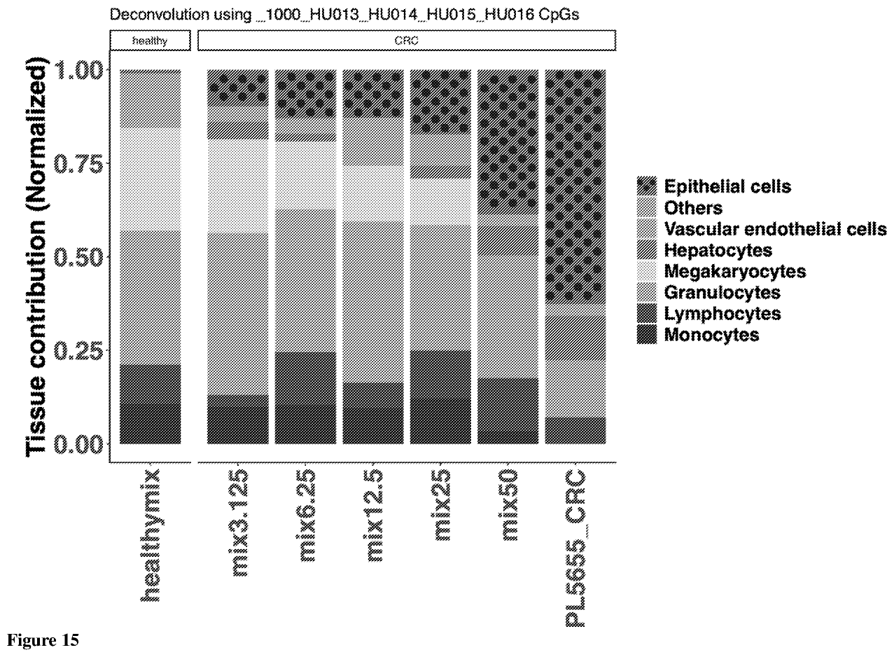

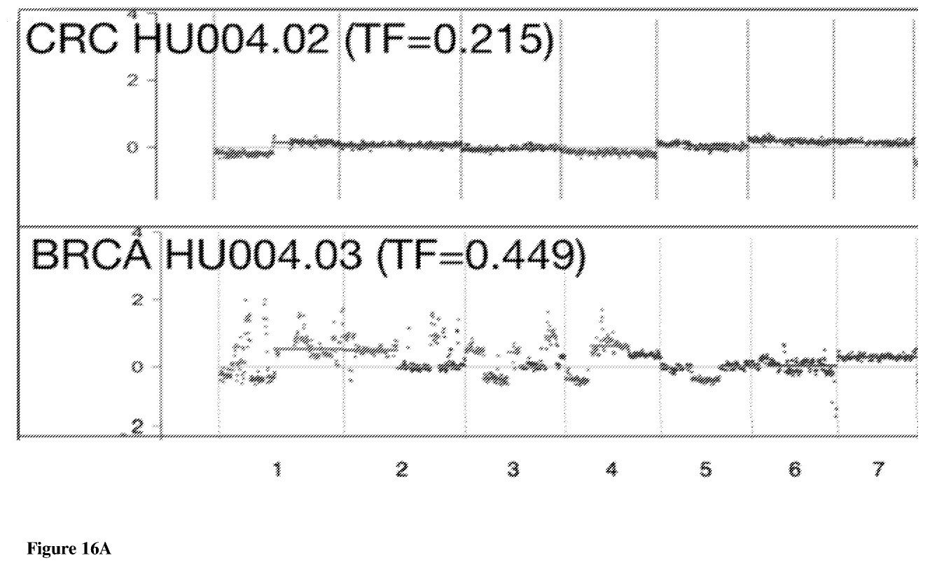

HU Plasma cfDNA samples, library construction, and sequencing. For HU (Hebrew University) healthy samples in FIGS. 1-19, cfDNA extracted from 4 mL plasma as described in Fox-Fisher et al. These samples are listed in Table 1 under production site “HU”. Barcoded libraries were created using the NBD-EXP104 and SQK-LSK109 kits as for ISPRO samples. They were sequenced on a single MinION flow cell, using standard MinKNOW runtime control (distribution v.21.11.7) without modification. For Hadassah Hebrew University samples in FIGS. 15-19, these were collected from 4 mL plasma as described in cfDNA extracted from 4 mL plasma as described in Fox-Fisher et al. Barcoded libraries were created using the NBD-EXP104 and SQK-LSK109 kits. They were sequenced on MinION flowcells, and processed using standard MinKNOW runtime control (distribution v.21.11.7). These Hadassah Hebrew University samples are listed in Table 4.

2019 real-time basecalling and alignment of Nanopore fast5 files. Basecalling was done using “high-accuracy real-time” mode during the run using MinKNOW distribution v.18 for run S1, and v.19.06.9 Guppy version 3.0.6+9999d81 for the others. For multiplex runs, demultiplexing was performed with guppy (Version 5.0.16+b9fcd7b5b) using “--trim_barcodes--barcode_kits EXP-NBD104”. For the one singleplex sample (S1/19_326), adapters were trimmed using Porechop with parameters: “--discard_middle--extra_end_trim 0”. Minimap2 alignments were performed to GCF_000001405.39_GRCh38.p13 with minimap2 (Version 2.13-r850), using the parameters “-ax map-ont--MD”. The resulting BAM files were used for fragment length and fragment end motif analysis, below.

2022 High Accuracy Calling (HAC) basecalling and alignment of Nanopore fast5 files. HU and ISPRO Fast5 files were basecalled and demultiplexed with Guppy (Version 5.0.16+b9fcd7b5b) using “--flowcell FLO-MIN106--kit SQK-LSK109--trim_barcodes--barcode_kits EXP-NBD104”, model r9.4.1_450bps_hac. The resulting demultiplexed Fast5 files were used as input for Megalodon methylation analysis. Adapter trimming for singleplex sample and alignment was performed as for 2019 real-time Fast5 processing above, and the resulting BAM files were used for ichorCNA, nucleosome (CTCF), fragment length, and fragment end motif analysis, below.

Megalodon modification mapping to produce mod_mappings.bam files: Demultiplexed Fast5 files from the 2022 High Accuracy basecalling section above were processed using Megalodon v. 2.4.2 with the following command-line parameters “--edge-buffer 0--mod-min-prob 0--guppy-params ‘-d/usr/local/hurcs/guppy/6.0.1/data--barcode_kits EXP-NBD104--trim_barcodes’--remora-modified-bases dna_r9.4.1_e8 hac 0.0.0 5mc CG 0--guppy-config dna_r9.4.1_450bps_hac.cfg”. Internally, Megalodon used Guppy server version 6.0.1+652ffd1, and basecalling model r9.4.1_450bps_hac. By default, Megalodon filters out multi-mapping (supplementary) reads and uses the minimap2 “map-ont” mode to filter low quality mappings. Each individual Fast5 tile was run individually, and the resulting mod_mapping.bam files were merged into a single mod_mappings.bam file using samtools merge (v1.14). Samtools/HTSlib versions before v.1.14 can not handle the Mm/Ml modification tages. Because Megalodon reports only the reference sequence in the BAM records, and does not report any base substitutions, these are anonymous BAM files which do not contain any genetic information, and thus contain no personally identifiable information and can be shared publicly. These are the primary files used for all methylation analysis, described in more detail below, and are available from GEO GSE185307 and at Zenodo DOI: 10.5281/zenodo.6642503.

DeepSignal methylation calling and processing. We used DeepSignal Version 0.1.8 (4), with model “model.CpG.R9.4_1D.human_hx1.bn17 sn360.v0.1.7+/bn_17.sn_360.epoch_9.ckpt”, which was downloaded from the DeepSignal Google Drive (drive.google.com/open?id=1zkK8Q1gyfviWWnXUBMclwEDw3SocJg7P). For ISPRO samples. Fast5 were annotated with fastq from 2019 real-time basecalling; for HU samples. Fast5 were annotated with fastq from 2022 HAC basecalling. We used the DeepSignal call_mods (modification_call) output tsv file, extracting the (strand-specific) methylation calls for each CpG from column 9 (called_label field), and calculated a methylation beta value by taking the number of methylated reads (value 1) divided by the total number of reads (value 0 or value 1). These were collapsed into a bedgraph file with a value between 0-1 for every CpG covered. These are available as file “grouped-beta-value_bedgraph.zip” in GEO accession GSE185307 and at Zenodo DOI: 10.5281/zenodo.6642503. All genomic coordinates are in GRCh38 and are zero-based.

Extracting methylation beta values from Megalodon mod_mapping.bam files. Modification mapping by Megalodon to produce mod_mapping.bam files is described above. To extract (stranded) methylation information from the mod_mapping.bam files, we used modbam2bed (github.com/epi2me-labs/modbam2bed) v.0.4.5, specifying a minimum probability threshold of 0.667, and filtering out positions with 0 confident reads using awk. The full command line was “modbam2bed--cpg-t 4-a 0.333-b 0.667|awk ‘($5>0) {print} >out.bed”. All coordinates are in GRCh38 and are 0-based. These files are named “*0.5mC.cut0.667.hg38.bed.gz”. Column 11 corresponds to the percent of reads methylated. Modbam2bed does not provide a column for the actual number of reads that this percentage is based on, but it can be calculated from the other columns. readCount=(col5*col10)/1000. We also provide a simple bedgraph with just the methylation fraction (beta) values in files named “*cut0.667.hg38.sorted.bedgraph.gz”. These can be loaded into any genome browser. Both file types are available in GEO accession GSE185307 and at Zenodo DOI: 10.5281/zenodo.6642503

Mapping of cfNano methylation data to HM450k probes. Using the zero-based stranded bed files from modbam2bed (“5mC.cut0.667.hg38.bed.gz” files), we mapped each CpG covering either the forward or reverse strand of each CpG on the Infinium 450 k array. For each modbam2bed stranded column, we first got the readCount as (col5*col10)/1000. We then multiplied the methylation percentage by the read count for each strand to get the number of methylated reads. Then we divided the sum of the methylated read counts by the sum of the total read counts to get the unstranded percent methylation (beta value).

Methylation calling from external WGBS datasets. For Fisher-Fox et al., methylation “beta.gz” files were obtained from GSE186888, and processed as recommended using wgbs_tools (github.com/nloyfer/wgbs_tools) beta2bed function to obtain fraction methylated and read count for each CpG. For Nguyen et al., bed files with methylation fractions and read counts were obtained from Figshare 10.6084/m9.figshare.16817941.v1.

For Sun et al., we obtained fastq files from EGAD00001001602 and aligned using Biscuit (github.com/huishenlab/biscuit) v.0.3.15.20200318 using the command line “biscuit align-t 16 hg38.fa CTR153.fq.gz-b 1” piped into samblaster Gregory G. Faust and Ira M. Hall, “SAMBLASTER: Fast Duplicate Marking and Structural Variant Read Extraction”, Bioinformatics 30, no. 17 (Oxford Academic, 2014) hereby incorporated by reference in its entirety. v.0.1.24 to mark and remove duplicates with the command line “samblaster-i stdin -o stdout-M--exclude Dups--addMateTags--ignoreUnmated-d CTR153.hg38_discordant.sam-$ CTR153.hg38_split.sam--maxSplitCount 2--maxUnmappedBases 50--minIndelSize 50--minNonOverlap 20-u CTR153.hg38_.fastq--minClipSize 20”.

Methylation coverage downsampling. To downsample methylation coverage from bed files with read count and fraction methylated columns, we used a custom Perl script in the github.com/methylgrammarlab/cfdna-ont repository called downsampleMethylBed.pl. This script treats each read at each CpG as an independent observation, and then randomly samples from these until it has enough observations to reach the average genomic coverage requested. To obtain the coverage levels shown in FIG. 1, it was run with the command line “downsampleMethylBed.pl--coverageLevels 1E-3,2E-3,5E-3.1E-2,2E-2.5E-2,1E-1,2E-1,5E-1.1E0.2E0,5E0,1E1,2E1,5E1,8E1--fracTotalFieldsFrom0-3,4--ncpgsGenome 28217005”.

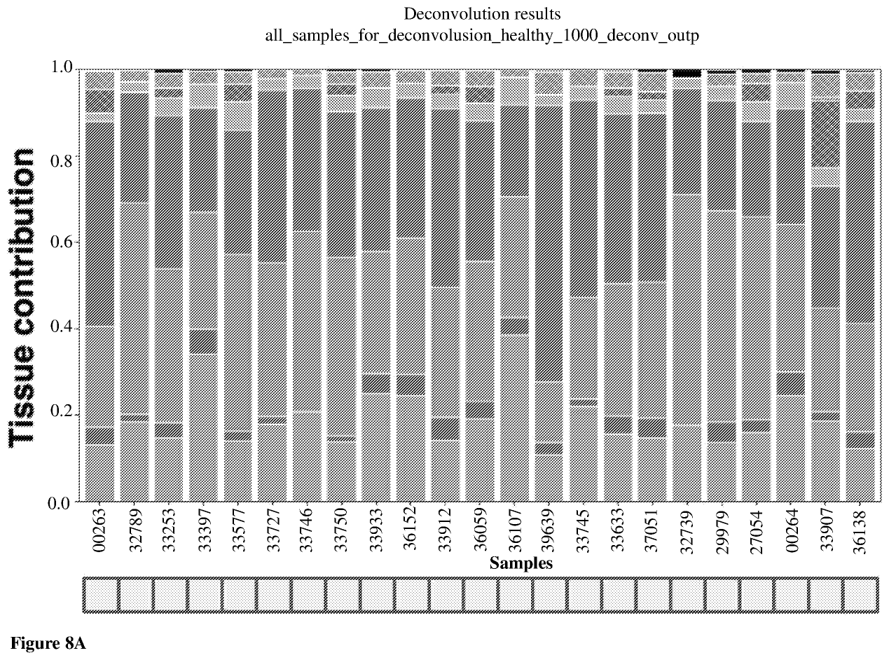

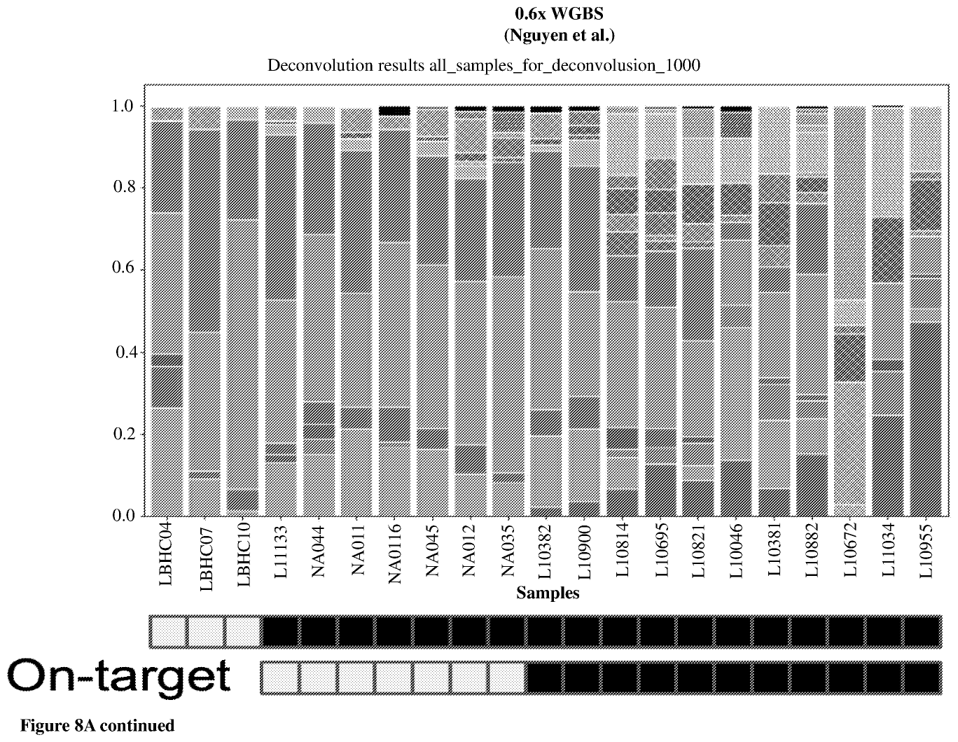

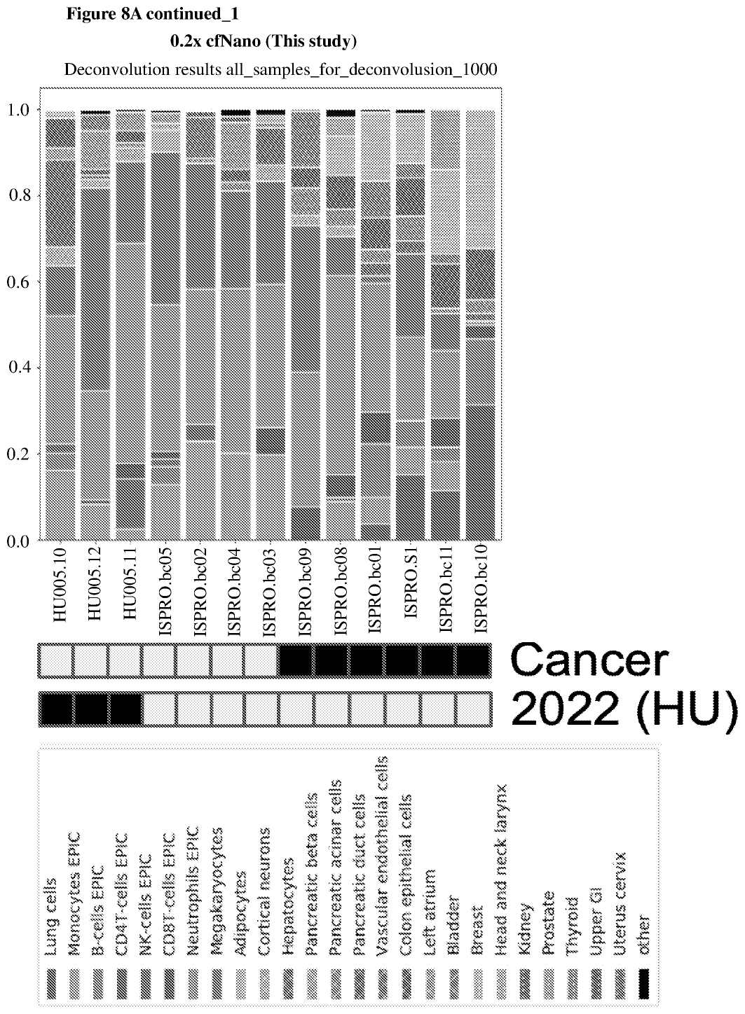

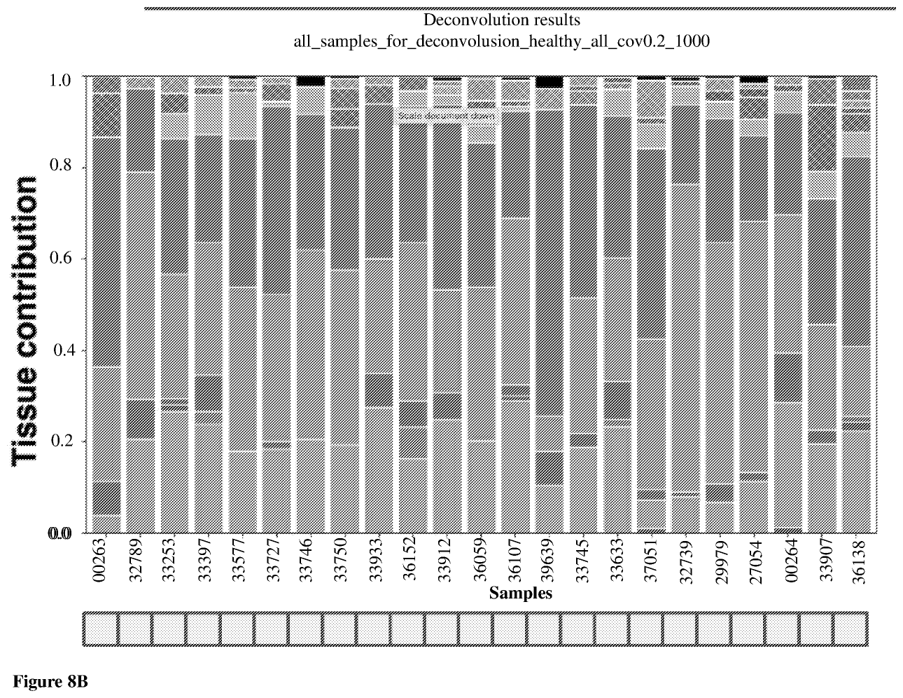

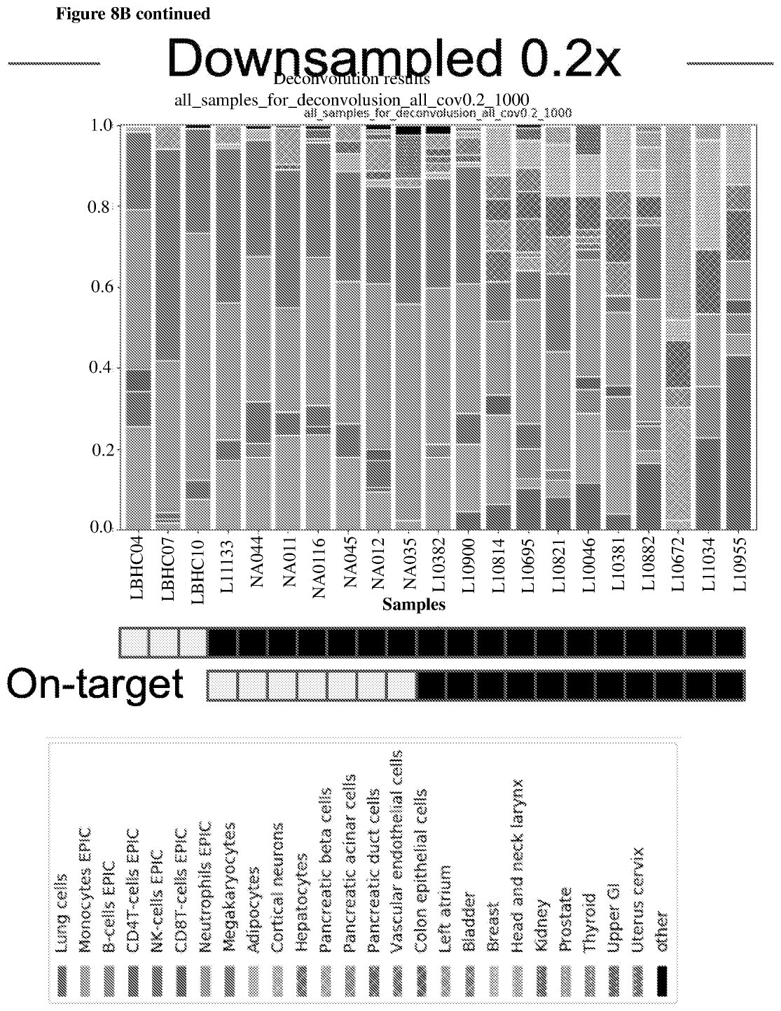

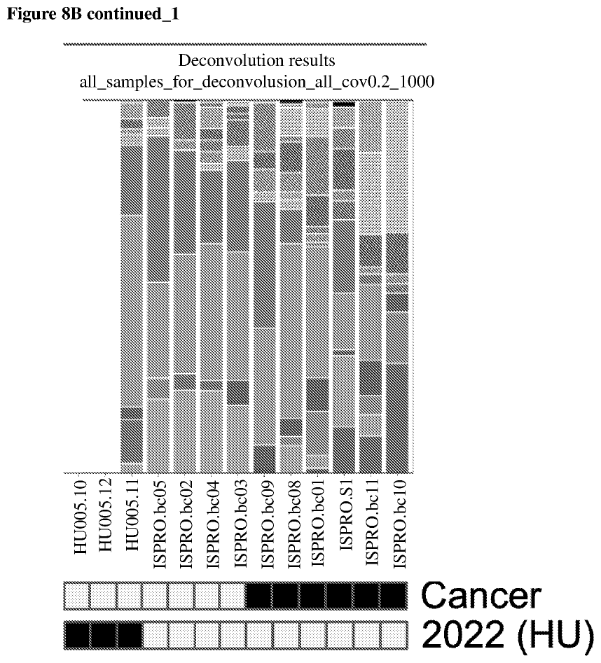

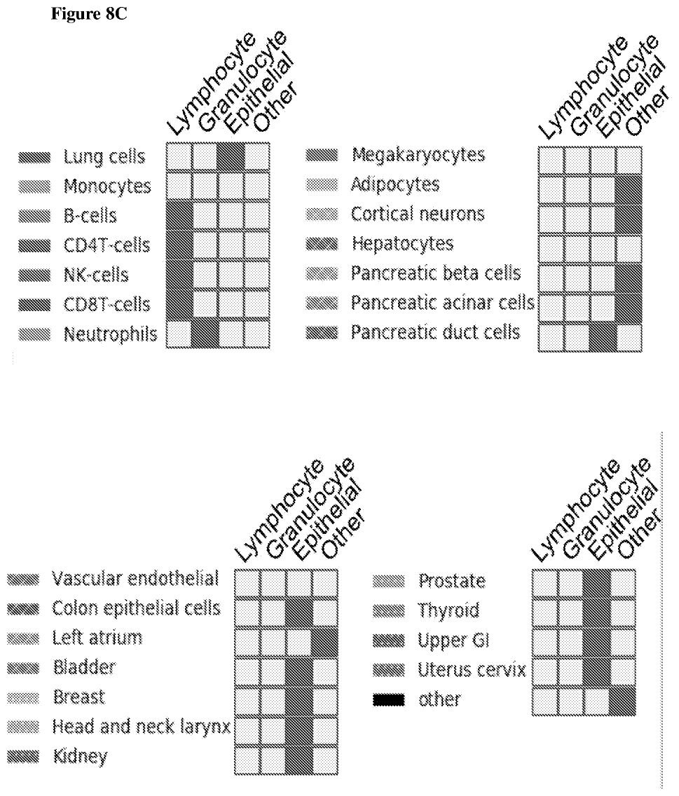

Full cell type methylation deconvolution. For the full cell type deconvolution in FIGS. 1A-C, we used the non-negative least squares regression (NNLS) method from Joshua Moss et al., “Comprehensive Human Cell-Type Methylation Atlas Reveals Origins of Circulating Cell-Free DNA in Health and Disease”, Nature Communications 9, no. 1 (2018), hereby incorporated by reference in its entirety. Specifically, we used the code from github.com/nloyfer/meth_atlas/blob/master/deconvolve.py. We used an input set of methylation markers that included the top 1,000 hypermethylated and 1,000 hypomethylated CpGs for each of the 25 cell types provided. To generate the reference atlas, we used the script github.com/methylgrammarlab/cfdna-ont/blob/main/deconvolution_code/cell_type_probes/creating_reference_atlas/feature_sele ction_function.m, with the input of “1000” as number of CpGs. Full results were plotted using a modified version of the original deconvolve.py which we have deposited here: github.com/methylgrammarlab/cfdna-ont/blob/main/deconvolution_code/deconvolution_moss/plot_deconv.py. These are shown in FIG. 8A-8B.

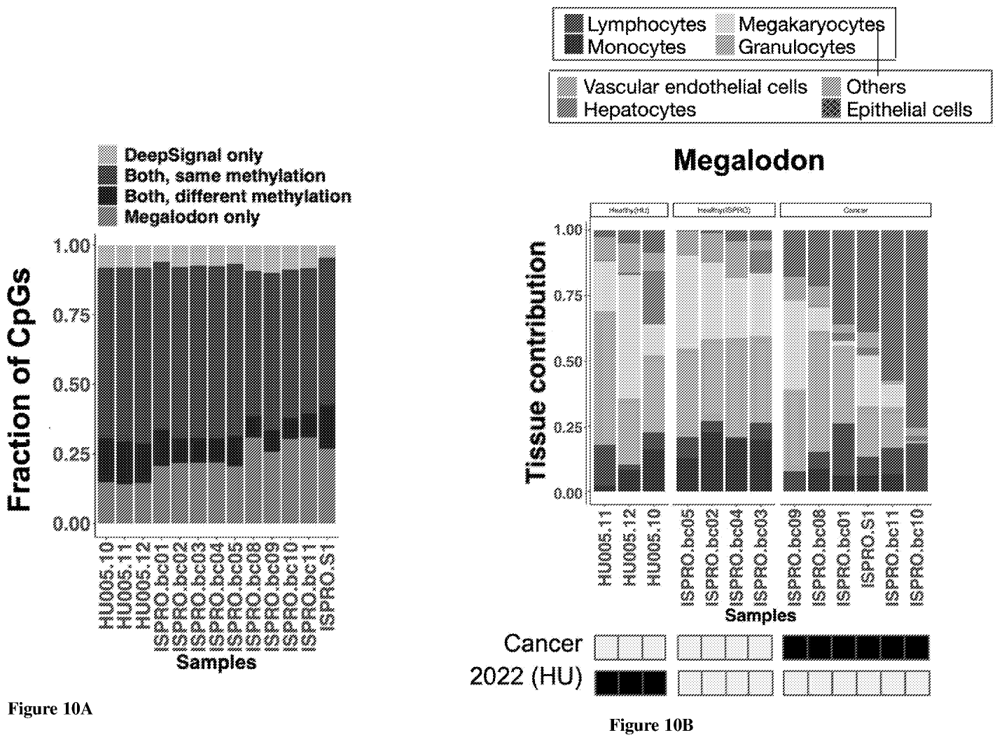

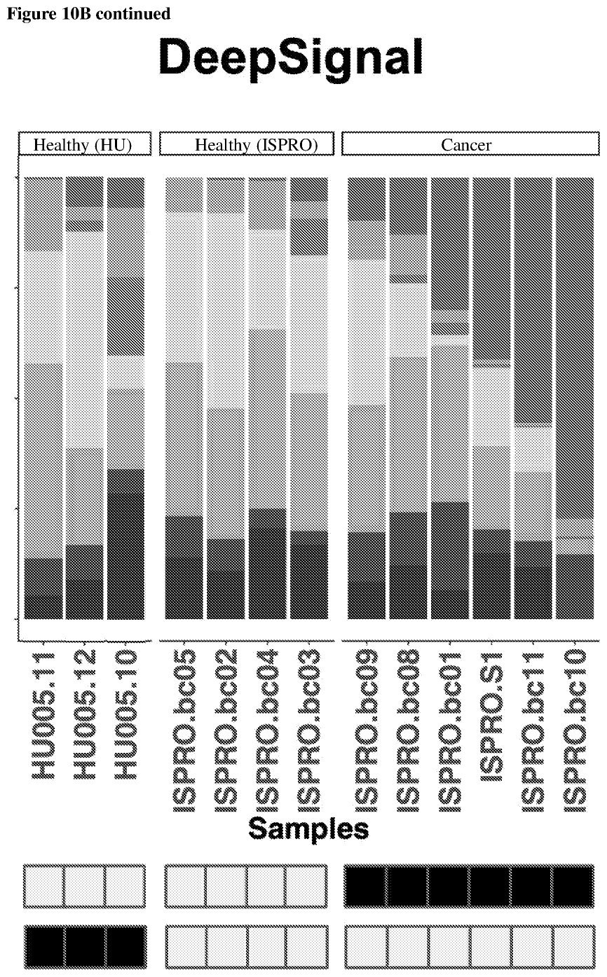

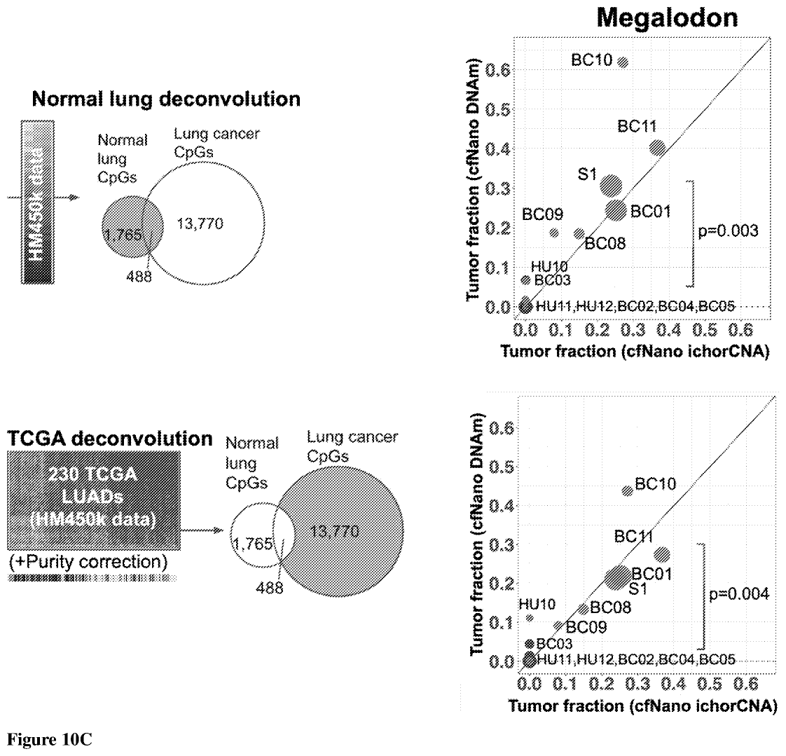

For FIGS. 1A-1C, we collapsed cell types into 8 groups using the file github.com/methylgrammarlab/cfdna-ont/blob/main/deconvolution_code/deconvolution_moss/group_file_for_plot_green_epithil ial.csv (shown visually in FIG. 8C). We plotted results using code in github.com/methylgrammarlab/cfdna-ont/blob/main/deconvolution_code/deconvolution_moss/deconvolution_plot.R. For DeepSignal methylation data, the procedure was the same except we used the top 2,000 hypermethylated and top 2.000 hypomethylated CpGs, to account for the significantly smaller number of CpGs called in the DeepSignal data (shown in FIG. 10A).

ichorCNA analysis. BAM files from the 2022 HAC basecalling and alignment step above were used as input. Samtools (Version 1.9) was used to filter BAM alignments, unmapped reads, secondary and supplementary reads, reads with mapping quality less than 20 as in Timour Baslan et al., “High Resolution Copy Number Inference in Cancer Using Short-Molecule Nanopore Sequencing”, BioRxiv, Dec. 29, 2020, hereby incorporated by reference in its entirety, and reads longer than 700 bp. For Illumina alignments we trimmed all ‘N’ nucleotides from the 3′ ends of fastq data, alignments were performed to GCF_000001405.39_GRCh38.p13 with BWA mem (43), duplicates were marked using picard MarkDuplicates and removed with samtools; read pairs without the properly-paired flag were removed.Pipelines used for preprocessing and filtering of both Nanopore and Illumina data are available at github.com/Puputnik/Fragmentomics_GenomBiol. Somatic copy number analysis was performed using the ichorCNA package v.0.3.2 Viktor A. Adalsteinsson et al., “Scalable Whole-Exome Sequencing of Cell-Free DNA Reveals High Concordance with Metastatic Tumors”, Nature Communications 8, no. 1 (2017), hereby incorporated by reference in its entirety.

We used ichorCNA to determine copy number alterations and tumor fraction for each cancer sample. If the percentage of genome covered by CN alterations was less than 15%, then the tumor fraction was determined to be unstable and set to 0. The ichorCNA parameters were (available within submitted source code) is “--ploidy c(2)--normal c(0.5)--maxCN 7 --includeHOMD False--estimateNormal True--estimatePloidy True--estimateScPrevalence True--altFracThreshold 0.001--rmCentromereFlankLength 1000000”.

Two-component cell type methylation deconvolution using healthy lung epithelia. To determine lung fraction specifically from different datasets, we devised a “two-component” version of the NNLS regression model described above. To compose the atlas of differentially-methylated probes in 25 human tissues and cell types, we used the data collected and tissue-specific feature selection method from the MethAtlas package (github.com/nloyfer/meth_atlas) of Moss et al., The script feature_selection.m was used to select Lung_cell epithelial specific CpGs. For the Megalodon version, the cutoff was set to select the top 1,000 hypermethylated and the top 1,000 hypomethylated probes, for the three Lung_cell epithelia samples vs. the four healthy plasma cfDNA samples from Moss et al., For DeepSignal methylation data, the procedure was the same except we used the top 2,000 hypermethylated and top 2.000 hypomethylated CpGs, to account for the significantly smaller number of CpGs called in the DeepSignal data (shown in FIG. 10A). We removed any probe that did not have valid (non-NA) values for 2 or more of the Lung_cell samples and 2 or more of the healthy plasma samples.

For each probe, the 450 k beta values were averaged to produce a single Lung-specific beta value X₁. The same was done for the four plasma cfDNA samples from to yield a healthy cfDNA beta value X₂. We used the Lawson-Hanson algorithm for non-negative least squares (NNLS) (cran.r-project.org/web/packages/nnls) to perform non-negative least squares regression as in Moss et al., Specifically, we identified non-negative coefficients β₁ and β₂, representing the fraction of Lung cells and normal blood cells in the Nanopore cfDNA mixture, respectively, subject to the constraints argminβ∥Xβ-Y∥₂ and β≥0. Then a single Lung fraction B was determined by having B1 and 62 sum to 1, with the equation

$$β = \frac{{β}_{1}}{( {β}_{1} + {β}_{2} )} .$$

Two-component cell type methylation deconvolution using TCGA lung tumors. We downloaded the Infinium 450 k beta value files for TCGA Lung Adenocarcinoma (LUAD) tumors using the ELMER packaged in Bioconductor Tiago C. Silva et al., “ELMER v.2: An R/Bioconductor Package to Reconstruct Gene Regulatory Networks from DNA Methylation and Transcriptome Profiles”, Bioinformatics 35, no. 11 (2019), hereby incorporated by reference in its entirety. We removed any probe that did not have valid (non-NA) values for 2 or more of the LUAD samples and 2 or more of the healthy plasma samples. In order to make this analysis completely independent from the healthy lung epithelia deconvolution analysis, we excluded 488 that were in the 2,000 probe set for the Megalodon healthy lung analysis, and an additional 396 that were in the 4,000 probe set for the DeepSignal healthy lung analysis (described above). We then performed a t-test to compare the methylation beta values of these LUAD specific probes to the four plasma cfDNA samples from the MethAtlas paper Moss et al., requiring a Benjamini-Hochberg corrected FDR of <0.001 and an absolute beta value difference of 0.3 or greater.

Correcting TCGA methylation model for cancer cell purity. NNLS was performed as above for the TCGA lung tumor deconvolution, with the following adaptation. The deconvolution assumes that each of the reference cell types is a representation of the purified cell type, but this is not the case for bulk TCGA tumors which have a median of leukocyte fraction of 30%. For each probe in each TCGA cancer sample, we corrected for this by solving for the equation Mₘ=M_{c}β+Mₗ(1−β), where Mₘ is the methylation of the mixture. M_{c} is (unknown) methylation of the cancer cells, Mₗ is the (known) methylation of the leukocytes, and β is the (known) percentage of cancer cells in the mixture. Mₗ was taken as the average of white blood cell samples from the MethAtlas of Moss et al. and β was taken as the “tumor purity” estimate based on somatic copy number alterations from the TCGA PanCan Atlas project using the ABSOLUTE program downloaded from the PanCan Atlas website (TCGA_mastercalls.abs_tables_JSedit.fixed.txt, gdc.cancer.gov/about-data/publications/pancanatlas). We used the pure cancer cell estimates Me, and performed NNLS regression as described above.

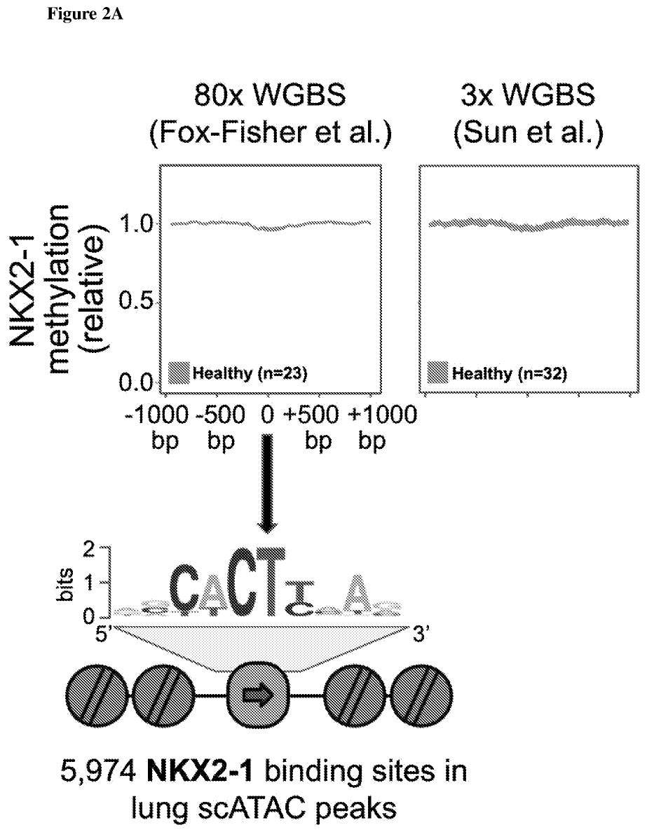

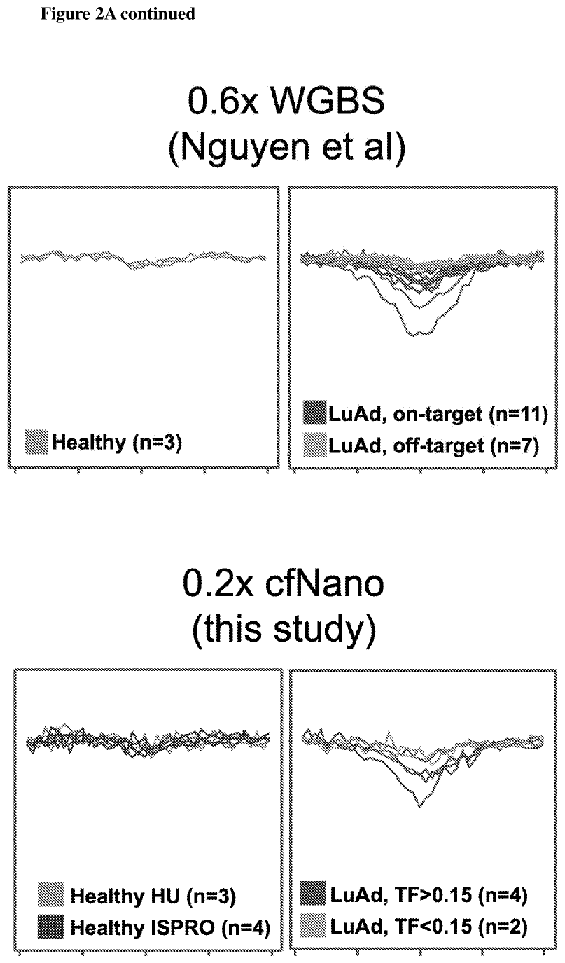

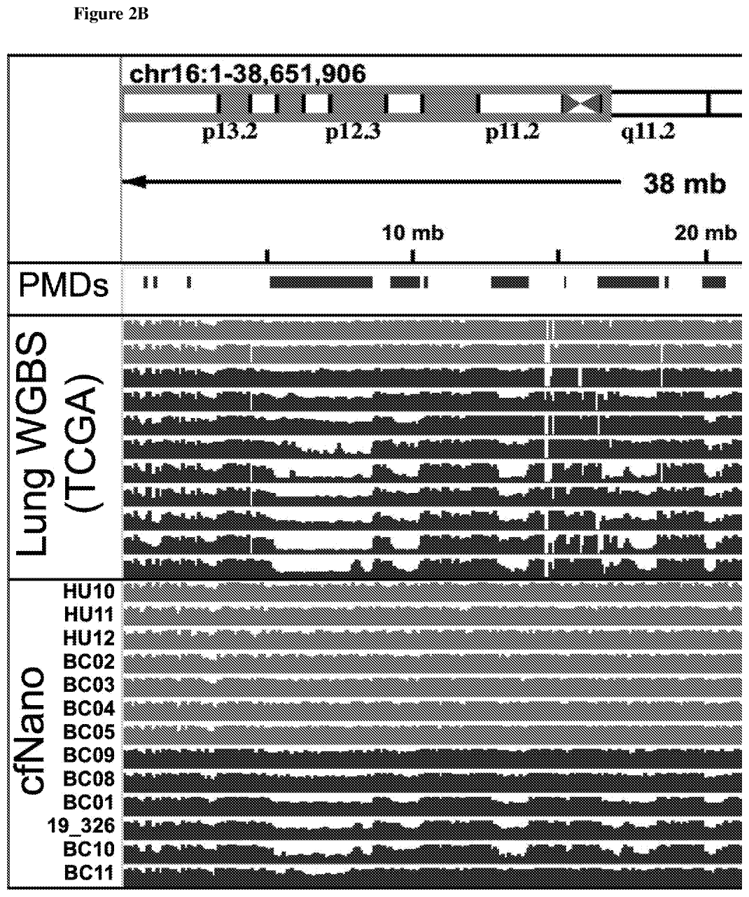

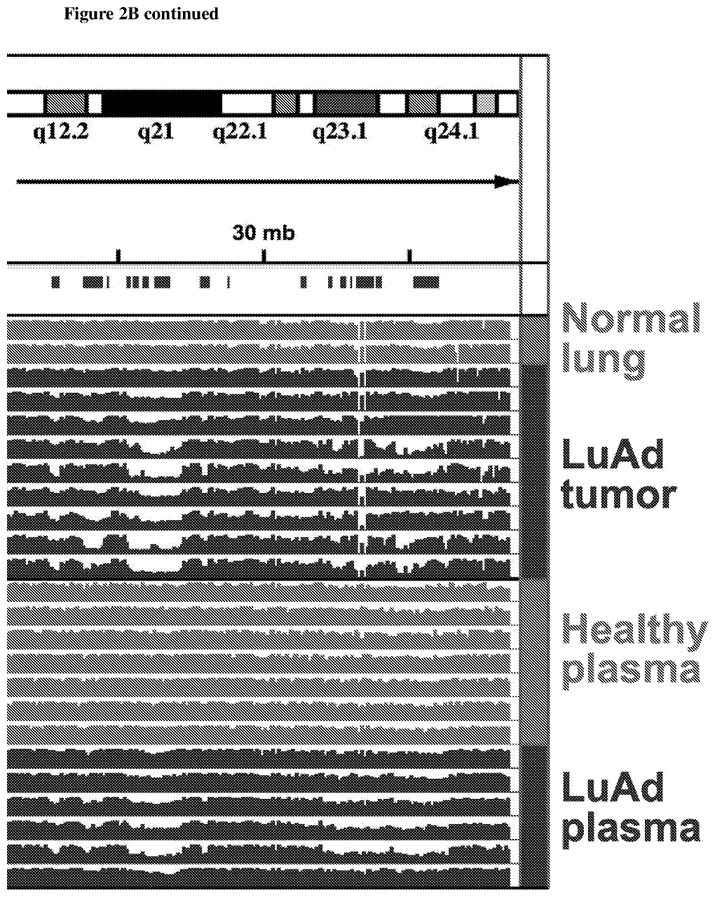

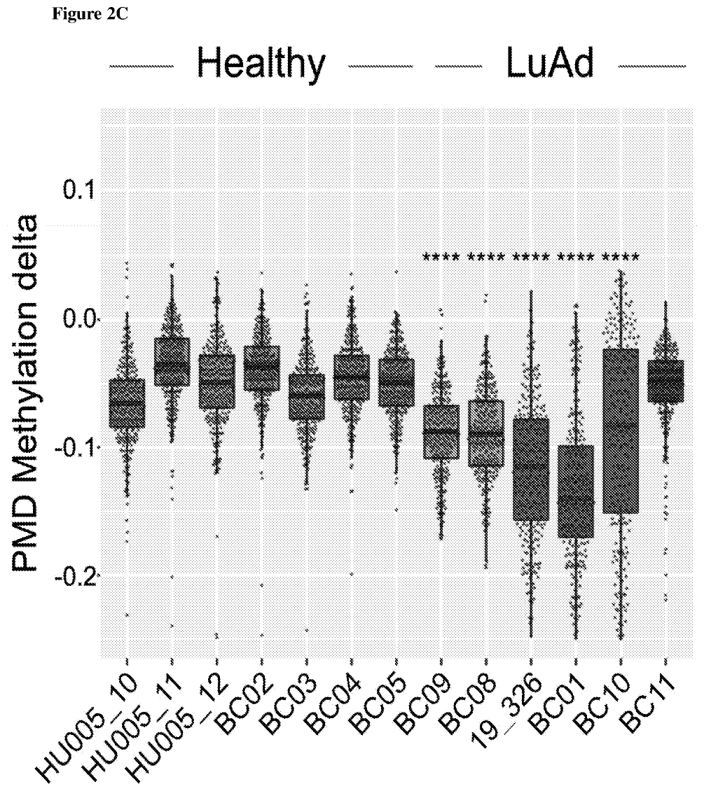

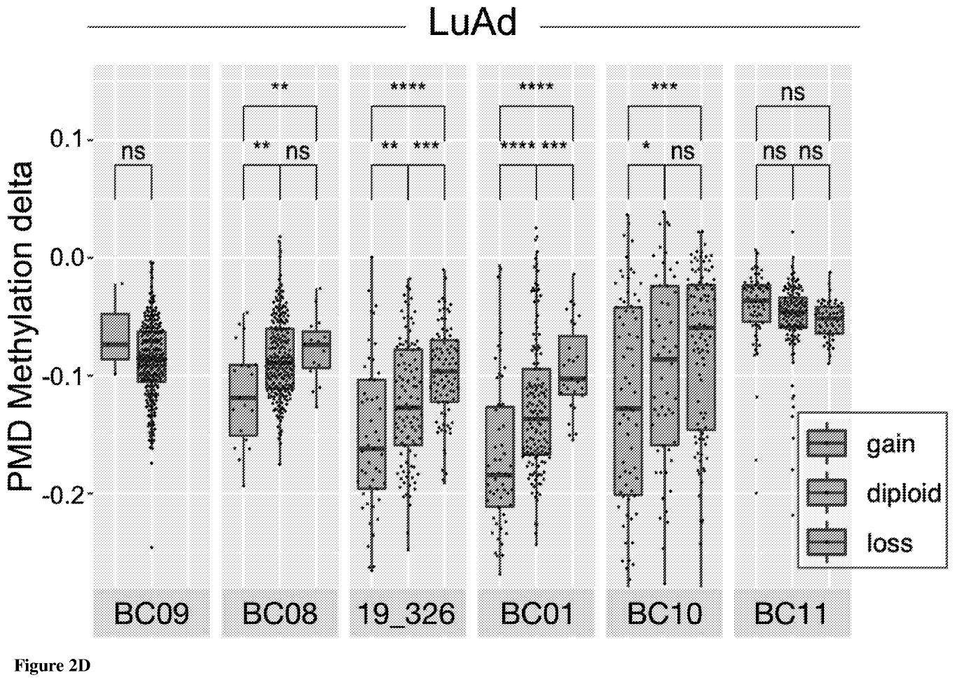

DNA methylation in 10 Mbp PMD bins. To generate FIGS. 2D-E. GRCh19 segmentation results from Martignano et al. were divided into non-overlapping 10 Mb bins. Copy number status of each bin was determined by log 2 ratio segment mean >0.10 and <-0.10 for Gain and Loss respectively. For the healthy samples, 10 Mb bins were generated from the whole genome. GRCh38 Methylation files were converted to GRCh37 using liftover R package. We selected only the bins overlapping one or more common Partially Methylated Domains (PMDs) from Zhou et al. Within these PMD bins, we took the average of all “solo-WCGW” CpGs overlapping a PMDs, with “solo-WCGW” annotation also from Zhou et al. We calculated the bin average from these CpGs as sum (frac_methylation_each_CpG)/CpG_count. We then subtracted this bin average and subtracted it from the average of all CpGs in the genome, to get the Methylation Delta shown in FIG. 2C-D. Common PMD and solo-WCGW annotations were taken from file zwdzwd.s3.amazonaws.com/pmd/solo_WCGW_inCommonPMDs_hg38.bed.gz. Statistical significance between each cancer sample's bins and all pooled healthy sample bins in FIG. 2D was calculated by one-sided Wilcoxon test (because we decided a priori to look only for hypomethylation in the cancer samples). For copy number analysis in FIG. 2D, each pair of copy number groups was compared using a one-sided Wilcoxon test, to test the hypotheses that diploid should have higher methylation than amplified regions, and deleted regions should have the highest methylation. The files and pipeline used for this analysis are available at github.com/Puputnik/CNV_Methylation_Genome_Biol_2022.

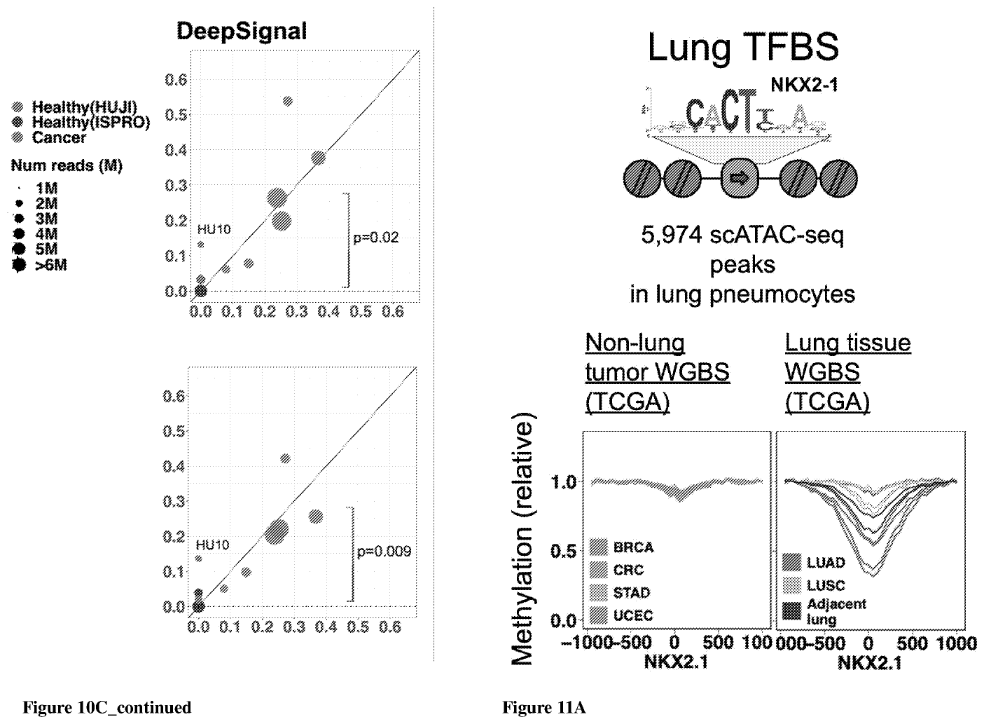

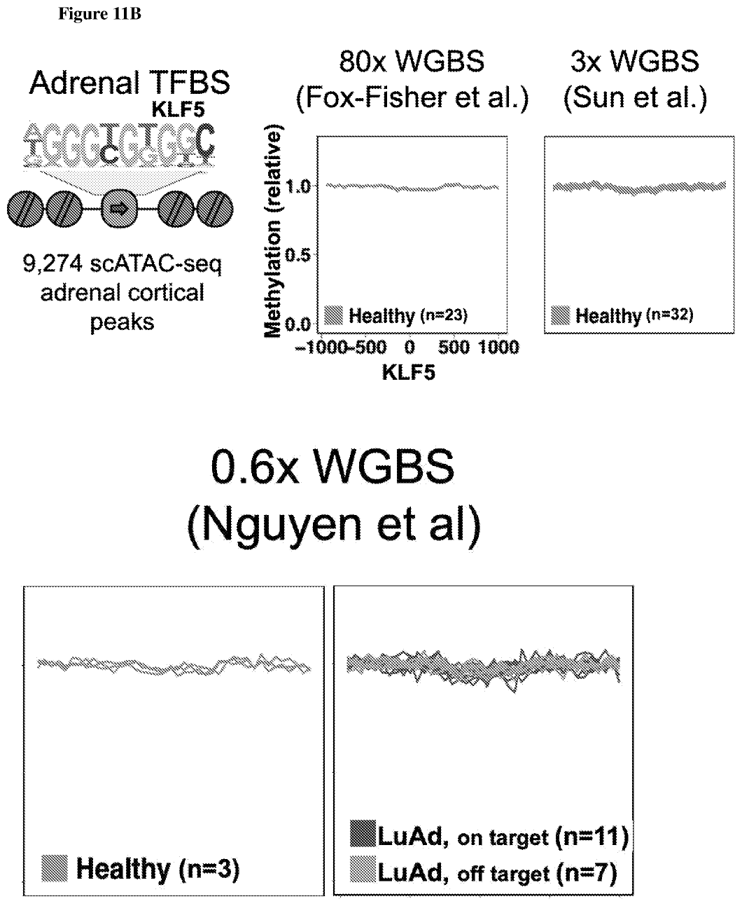

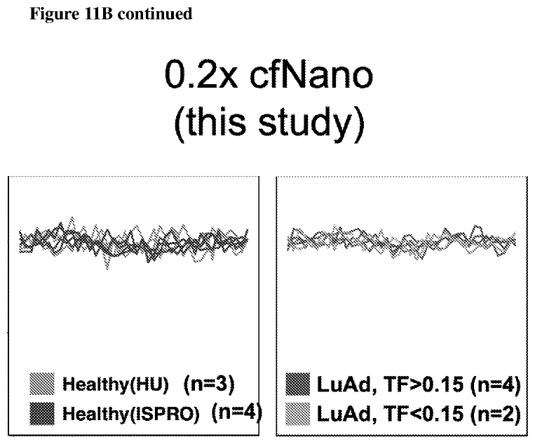

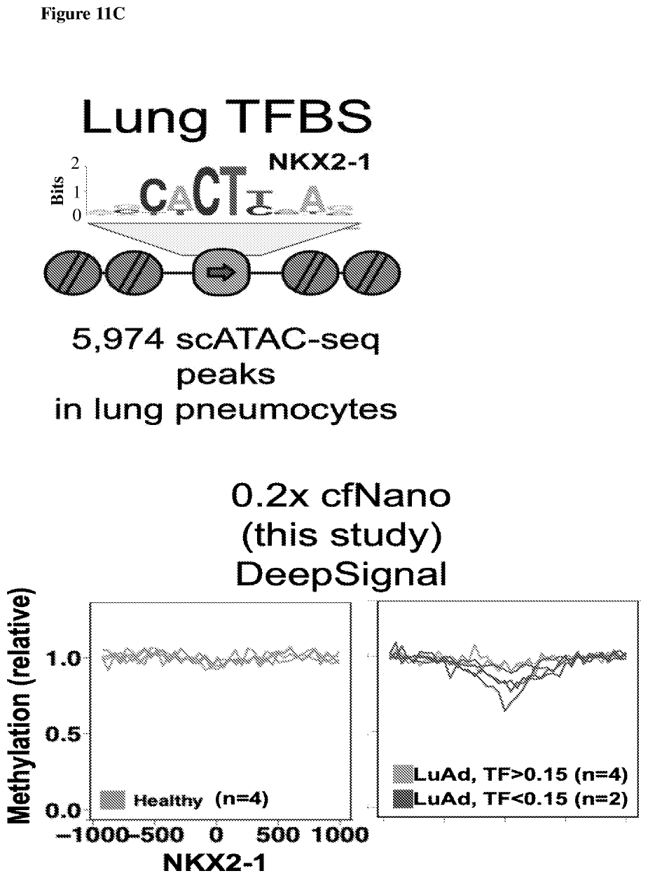

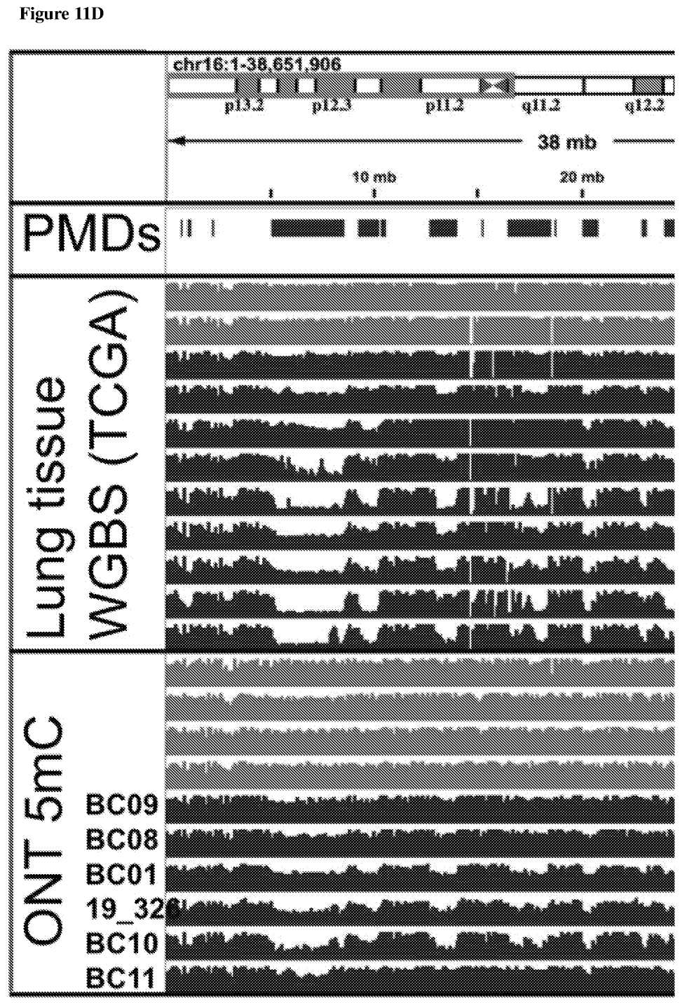

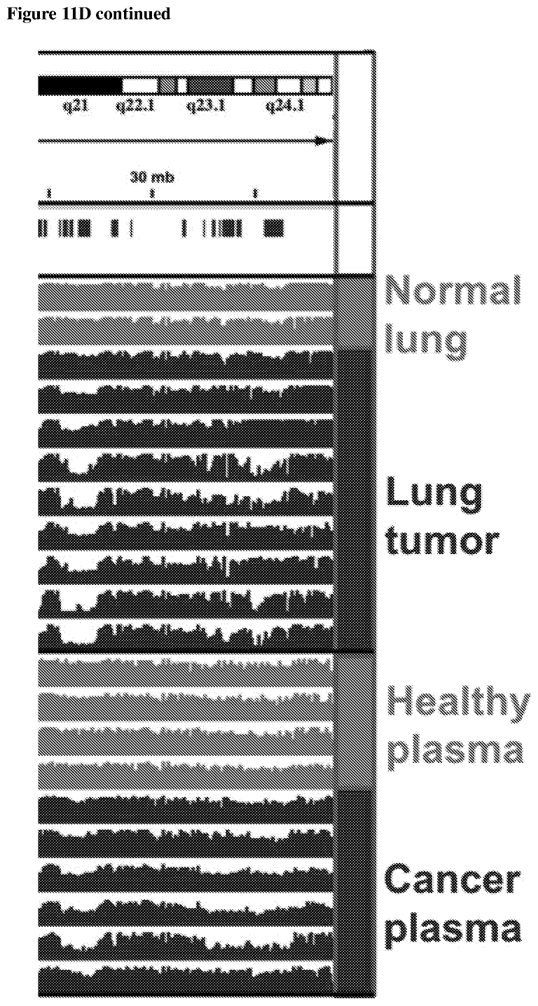

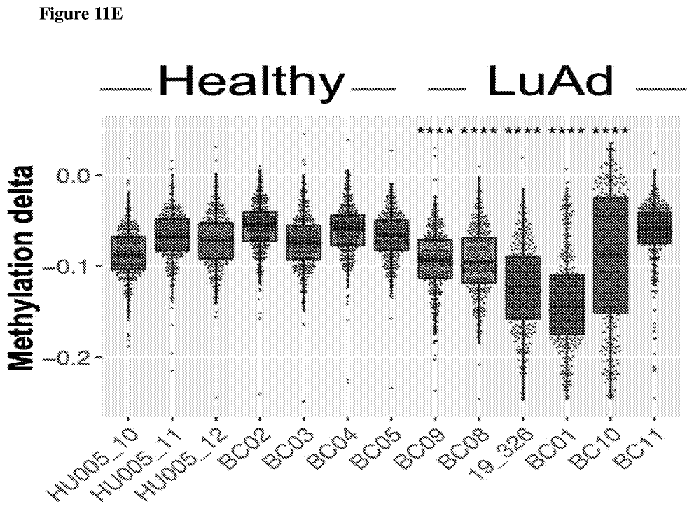

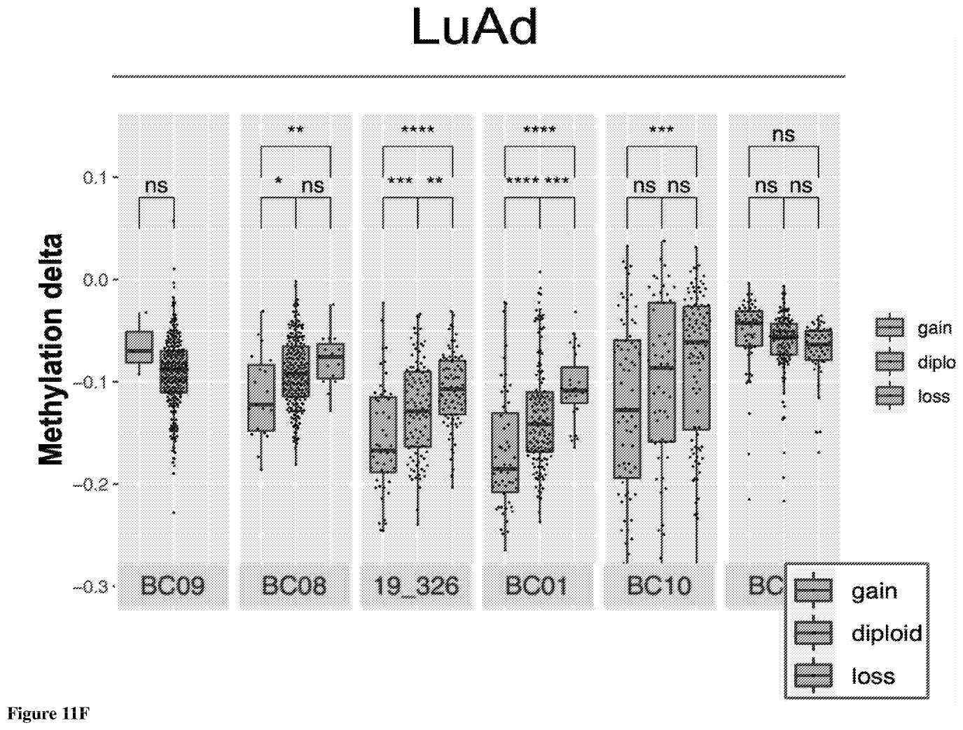

Transcription factor binding site (TFBS) analysis. First, we used HOMER to identify predicted NKX2-1 binding sites (using the HOMER built in matrix “nkx2.1.motif”) across the GRCh38 genome, and removed any site within the ENCODE blacklist. For normal lung cell analysis, we intersected this list with 6,754 ATAC-seq peaks identified in the pneumocyte (PAL) cluster 13 CREs from Kai Zhang et al., “A Cell Atlas of Chromatin Accessibility across 25 Adult Human Tissues”, BioRxiv (Cold Spring Harbor Laboratory, 2021), hereby incorporated by reference in its entirety (downloaded from supplemental table 6 of that paper “Table_S6_Union_set_of_cCREs.xlsx”). We then selected 5,974 peaks that overlapped a predicted NKX2-1 TFBS, and centered each on the predicted NXK2-1 TFBS. If multiple TFBS were present in the peak, we took the motif with the highest HOMER log-odds match score. This TFBS set is available as file “nkx2.1.incluster 13_distalPeaks_PAL.bed.highestScoreMotifs.hg38.bed” in GEO accession GSE185307 and at Zenodo DOI: 10.5281/zenodo.6642503. To calculate relative methylation levels, raw methylation levels in each bin were divided by the mean methylation within all bins from −1000 to −800 and +800 to +1000 across all NKX2-1 sites. For FIG. 11A, we used all WGBS cancer types that were represented by normal tissues in the scATAC-seq atlas, as this was the atlas used to define pneumocyte specific (PAL) peaks. For TCGA lung and non-lung samples in FIG. 11A, we downloaded TCGA WGBS bedgraph files from zwdzwd.github.io/pmd from Zhou et al., We used all WGBS cancer types that were represented by normal tissues in the scATAC-seq atlas, as this was the atlas used to define pneumocyte specific (PAL) peaks. These TGCA types included LUAD and LUSC (Lung tissue from atlas). CRC (Transverse colon tissue from atlas), BRCA (Breast tissue from atlas), STAD (Stomach tissue from atlas), and UCEC (Uterus tissue from atlas).

KLF5 transcription factor binding site (TFBS) analysis (FIG. 11B). As with NKX.2 above, we used HOMER to identify predicted KI.F5 binding sites (using the HOMER built in matrix “klf5.motif’) across the GRCh38 genome, and removed any site within the ENCODE blacklist. As a control, we intersected this list with 9,274 ATAC-seq peaks identified in the cluster 43 CREs from Zhang et al. (downloaded from supplemental table 6 of that paper “Table_S6_Union_set_of_cCREs.xlsx”). We then selected 1,762 peaks that overlapped a predicted KLF5 TFBS, and centered each on the predicted KLF5 TFBS. If multiple TFBS were present in the peak, we took the motif with the highest HOMER log-odds match score. This TFBS set is available as file “klf5.incluster43Distal.txt.highestScoreMotifs.bed” in GEO accession GSE185307.

CTCF nucleosome positioning analysis. We used 9,780 evolutionarily conserved CTCF motifs occurring in distal ChIP-seq peaks, which were taken from Kelly et al., Nanopore or Illumina fragments within the size range of 130-155 bp were used for fragment coverage analysis, with reads being extracted from BAMs as described above. These shorter mononucleosomal fragments showed similar nucleosomal patterns but gave higher spatial resolution than 156-180 bp fragments. Deeptools (Version 3.5.0) bamCoverage was used with the parameters “--ignoreDuplicates--binSize-bl ENCODE_blacklist-of bedgraph--effectiveGenomeSize 2913022398--normalizeUsing RPGC”. For Illumina WGS, we used the additional parameter “--extendedReads 145”. The bedgraph was converted to a bigwig file using big WigToBedGraph downloaded from UCSC Genome Browser. This bigwig file was passed to Deeptools computeMatrix with the command line parameters “reference-point --referencePoint center-out table.out”, and the table.out file was imported into R to create fragment coverage heatmap.

Fragment length analysis. BAM files from either the 2019 real-time basecalling and alignment, or 2022 HAC basecalling and alignment, above, were used as input. Samtools (Version 1.9) was used to filter BAM alignments, unmapped reads, secondary and supplementary reads, reads with mapping quality less than 20 as in Baslan et al., “High Resolution Copy Number Inference in Cancer Using Short-Molecule Nanopore Sequencing”, and reads longer than 700 bp. For Illumina alignments we trimmed all ‘N’ nucleotides from the 3′ ends of fastq data, alignments were performed to GCF_000001405.39_GRCh38.p13 with BWA mem (43), duplicates were marked using picard MarkDuplicates and removed with samtools.Pipelines used for preprocessing and filtering of both Nanopore and Illumina data, and analyzed data are available at github.com/Puputnik/Fragmentomics_GenomBiol. In addition, only reads with barcodes at both ends (obtained using the -require_barcodes_both_ends flag while demultiplexing) were used for fragment length analysis of the multiplexed samples (all except 19_326). Read identifiers of double-barcoded reads are available in the “doubleBarcodeIds” file in Zenodo DOI: 10.5281/zenodo.6642503. Reads with soft clipping at either the 5′ or 3′ ends were removed. Fragment length was calculated from the Minimap2 BAM CIGAR column by summing all counts. Short mononucleosome ratio was calculated as

$$\frac{{numfrags}_{100 - 150 bp}}{{numfrags}_{100 - 220 bp}}$$

(150 bp is the same cutoff for short fragments used in Mouliere et al.). Short dinucleosome ratio was calculated as

$$\frac{{numfrags}_{275 - 3250 bp}}{{numfrags}_{275 - 400 bp}}$$

(this was determined visually from FIG. 2D of publication Mouliere et al.).

End motif analysis. BAM files from 2019 real-time basecalling and alignment, or 2022 HAC basecalling and alignment above were used as input. Fragments and reads were processed and filtered as in fragment length analysis. For cfNano, we only used read end1 because end2 could occasionally not represent the actual end of the fragment. To avoid biases that would affect end motif analysis, we also removed reads with any soft clipping at end 1. The first 4 bases of each fragment were extracted and used for 4-mer analysis. To avoid errors in Nanopore base calling, these 4 bases were extracted from the reference genome. Motif frequency was calculated as

$$\frac{{numfrags}_{4 mer}}{{numfrags}_{total}} .$$

For top 25 motifs and ranking order in FIG. 4 and FIG. 14, we used R. W. Y. Chan et al., “Plasma DNA Profile Associated with DNASE1L3 Gene Mutations: Clinical Observations, Relationships to Nuclease Substrate Preference, and In Vivo Correction”. Files and pipelines used for Fragment length and end motif analyses are available at github.com/Puputnik/Fragmentomics_GenomBiol.

Statistical tests. Student's t-test for all sample comparisons where at least one test group had less than five samples, otherwise Wilcoxon test was used.

Cancer/healthy mixture dilution experiments (FIG. 15). For healthy samples (Table 4, samples HU012.01-HU012.11): cfDNA samples from 11 healthy women (Ages 19-50) were individually barcoded and pooled for sequencing on a single flowcell. For mixing samples (Table 4, mix25, mix50, mix 12.5, mix 6.25, mix 3.125): cfDNA samples from same 11 healthy women were pooled (3 ng from each) and concentrated to a volume of 66 uL using 2.3×SPRI. This 33 ng cfDNA pool was used to represent healthy plasma (Table 1, “Hadassah healthymix”). First mixing sample (Table 4, “mix25”) was a mixture of 2 ng of cfDNA from tumor patient PL5655 (Table 4, “Hadassah PL5655”). and 6 ng from the healthymix cfDNA. The same 25% sample (“mix25”) was also used as a stock for 2-fold serial dilution with the healthy pool to produce 12.5%, 6.25%, 3.125% tumor cfDNA fractions. 50% sample was prepared separately by mixing 2 ng tumor cfDNA with 2 ng Healthy pool.

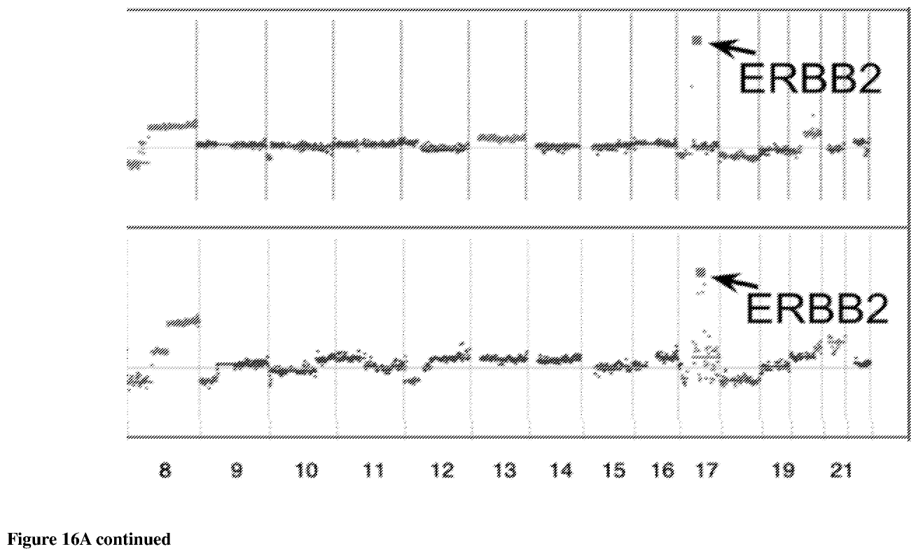

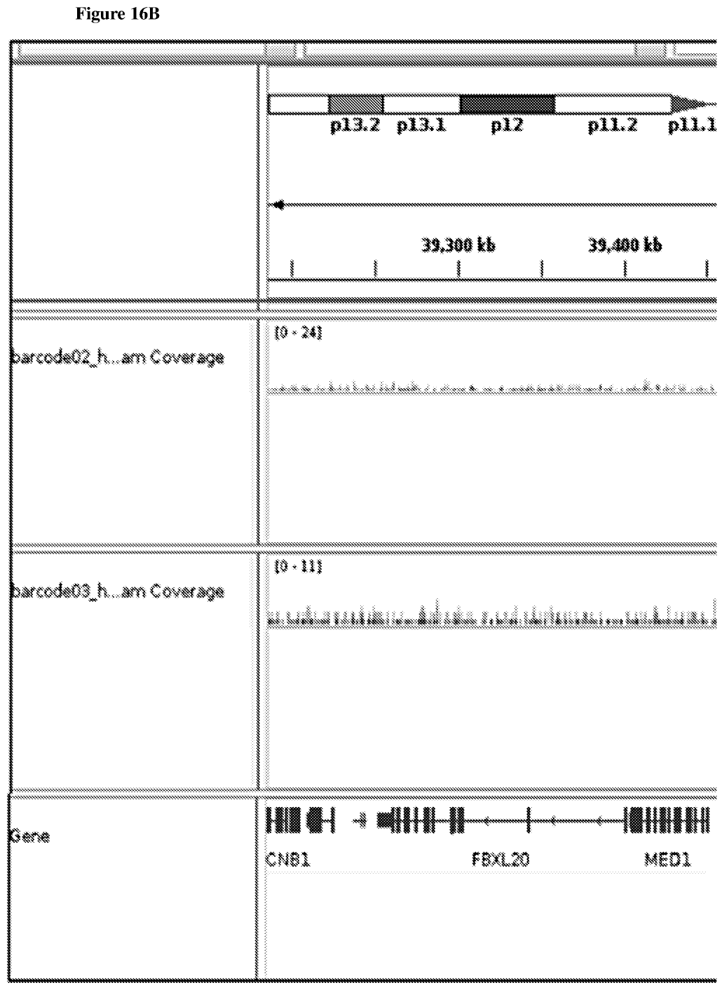

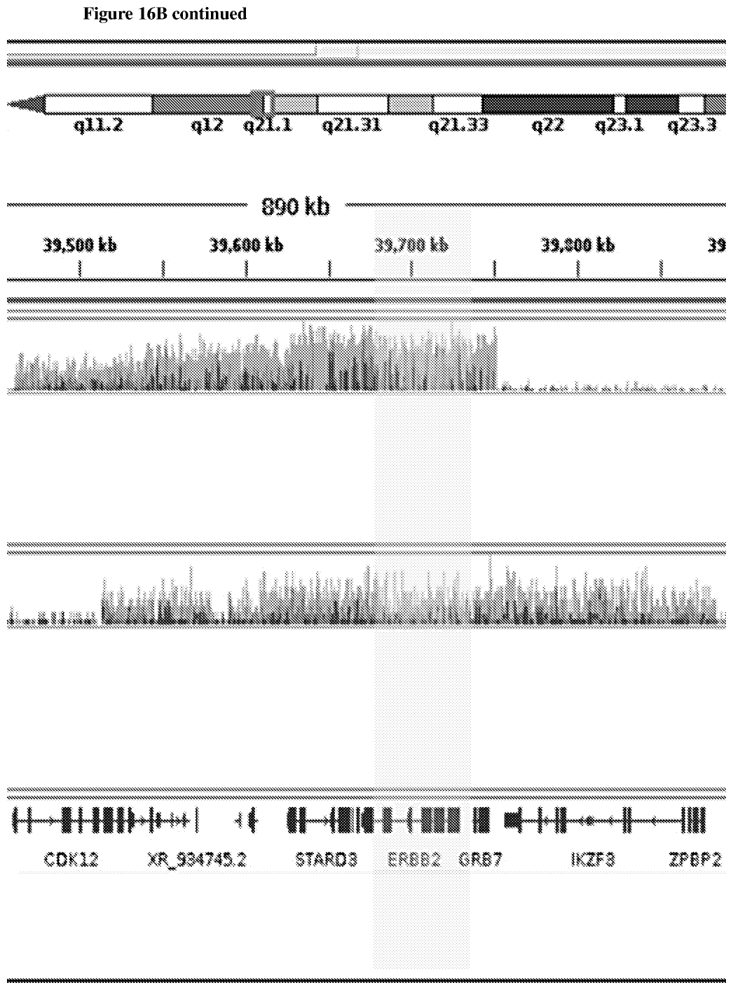

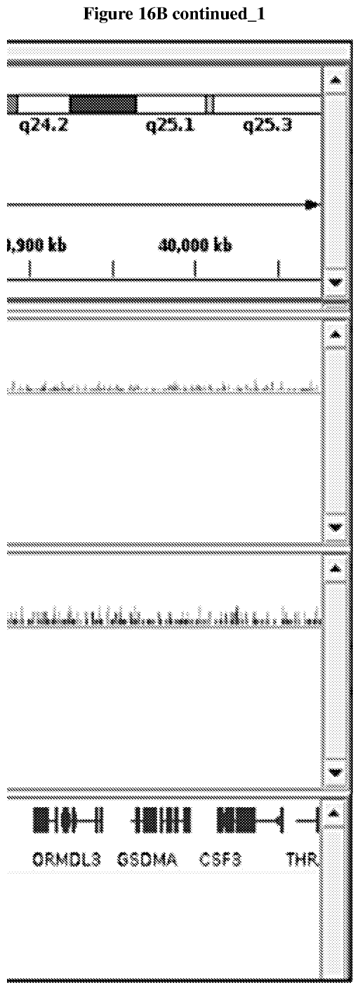

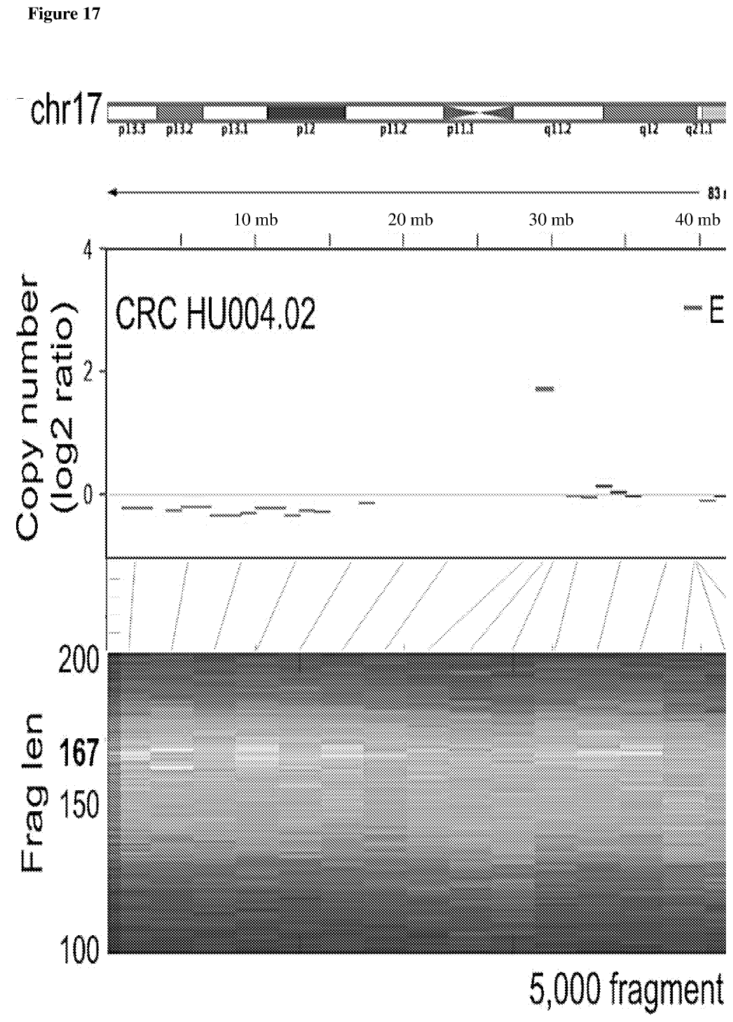

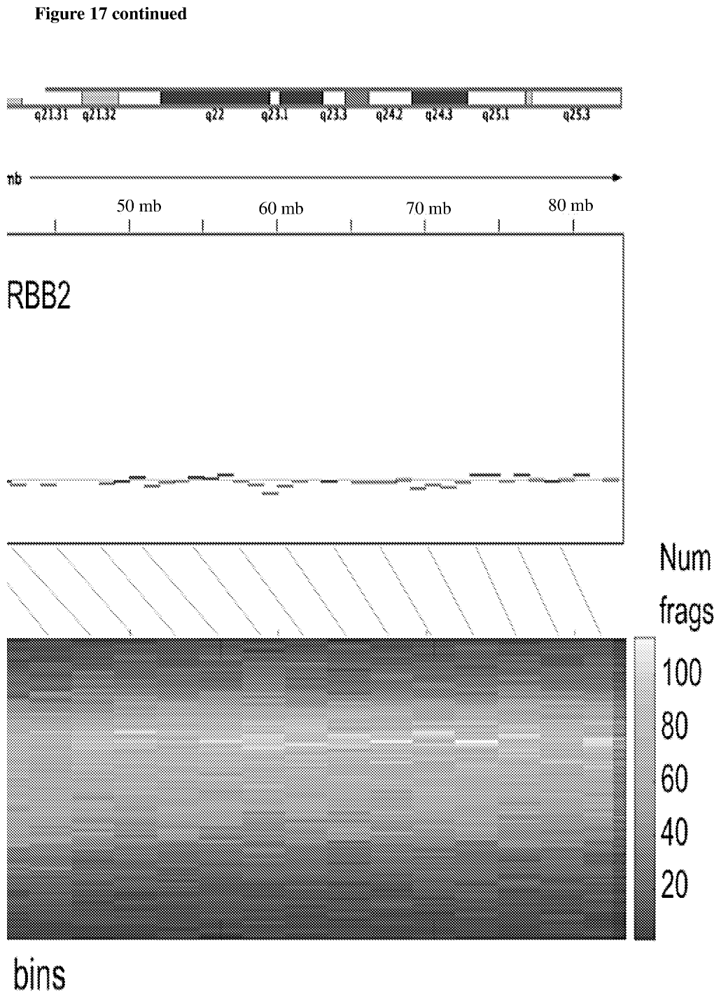

Combined copy number and fragment length analysis (FIGS. 16-17). We used ichorCNA analysis as described above in “ichorCNA analysis” to determine copy number alterations and tumor fraction in FIG. 16A. We used the Integrated Genome Viewer (IGV) to visualize read depth across the ERBB2 region in FIG. 16B. For FIG. 17, we used the copy number values from ichorCNA to plot copy number for 1-megabase bins along chromosome 17. We then started at the beginning of chromosome 17, and created evenly sized bins of exactly 5,000 reads (fragments), until reaching the end of chromosome 17. We mapped each of these to a 1-megabase bin by determining the 1-megabase bin that contained the largest number of the 5,000 reads. For each of these bins, we computed a histogram of the number of fragments from length 100 to 200, and plotted this histogram as a heatmap.

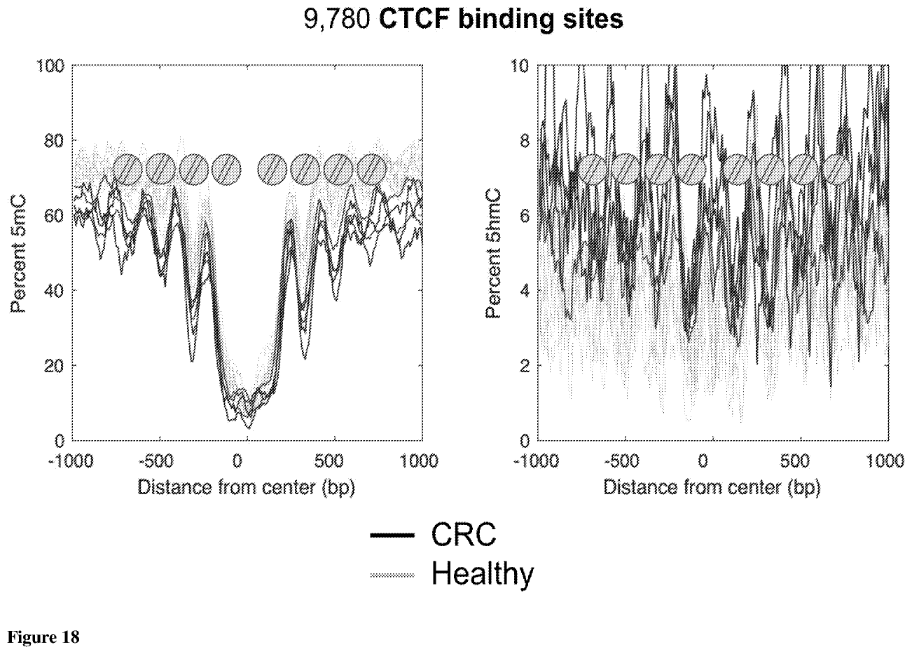

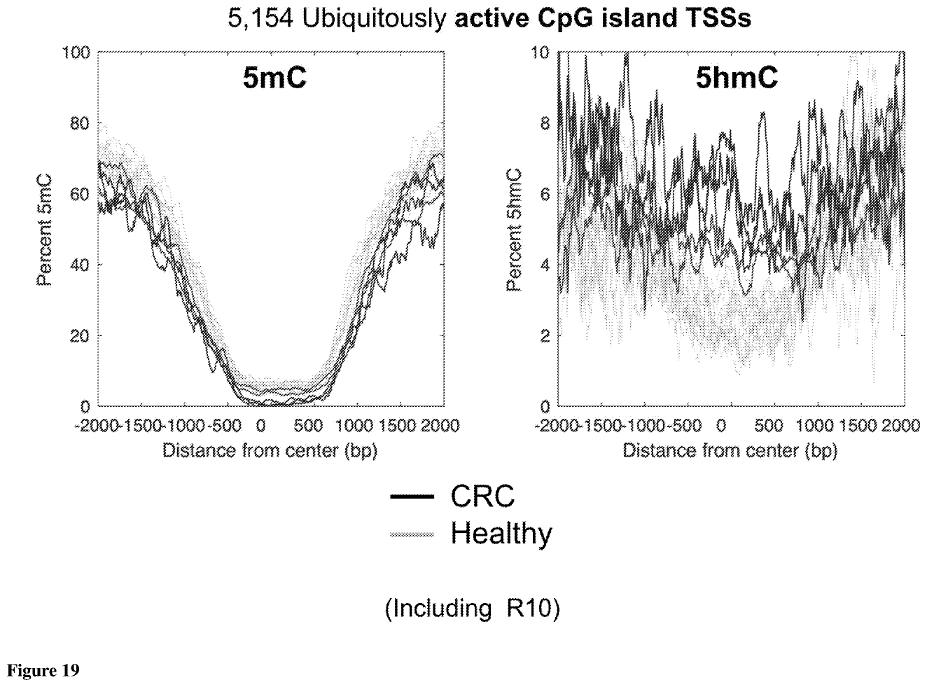

Combined 5-methylcytosine and 5-hydroxymethylcytosine analysis (FIGS. 18-19). All samples were processed with Megalodon, as described in the “Megalodon modification mapping to produce mod_mappings.bam files” and “Extracting methylation beta values from Megalodon mod_mapping.bam files” sections above, with the following changes. We used the “5hmc_5mC” Remora model dna_r9.4.1_e8, rather than the 5mC model. For modbam2bed, we used “-m 5hmC” for 5hmC, and “-m 5mC” for 5mC.

### Example 1: Estimating Cell Type Fractions from cfNano

We first performed cell type deconvolution of healthy plasma cfDNA using DNA methylation data from either published cfNano samples (Table 1) or published WGBS datasets (Table 2). For the published WGBS datasets, we used the methylation fractions (beta values) that were provided in the published data files. For our cfNano, we performed direct modification calling using the Megalodon software provided by ONT (github.com/nanoporetech/megalodon). To perform deconvolution, we used 1,000-2,000 marker CpGs per cell type based on a previously published atlas of purified cell types (“MethAtlas”, Moss et al., and Fox-Fisher et al.), and estimated cell type fractions using Non-Negative Least Squares (NNLS) regression as described in Moss et al., In order to better understand the impact of the relatively low sequencing depth of our cfNano samples (˜0.2× genome coverage), we first performed deconvolution of all samples using downsampling experiments starting with full sequence depth down to 0.0001× genome coverage (FIG. 1A and FIGS. 5-7). Healthy plasma WGBS samples were taken from a recent study of 50-100× genomic coverage (Fox-Fisher et al., FIG. 1A left “Fox-Fisher” samples), and another WGBS study with 0.5-1× coverage (Nguyen et al., FIG. 1A middle “Nguyen” samples). Finally, healthy cfNano samples were analyzed (FIG. 1A right “this study”). From full depth down to 0.2× (about 2.5M aligned fragments), all samples were dominated by the expected cell types: monocytes, lymphocytes, megakaryocytes, neutrophils/granulocytes, and sometimes hepatocytes. Cell type proportions became significantly degraded at 0.05× coverage and below (corresponding to less than 700,000 aligned fragments). The common cell types were consistently found across the 23 healthy individuals in the Fox-Fisher dataset, the 3 healthy individuals in the Nguyen dataset, and the 7 healthy individuals in the cfNano dataset, both at full depth (FIG. 1B) and when downsampled to 0.2× depth (FIG. 1C). The same was found when cell type groups, such as lymphocytes, were broken down into the 25 individual types (FIG. 8A-8C). Notably, a slight epithelial fraction was identified in some of the Fox-Fisher samples at 0.2×, which did not appear at full 80× depth, suggesting a small but measurable amount of noise at the 0.2× coverage level.

TABLE 1
cfNano samples from ISPRO Italy and Hebrew University Israel, processed using 5 mC modification calling

| | | | 2022 | 2022 HAC | 2022 | 2022 HAC | | | | |
|---|---|---|---|---|---|---|---|---|---|---|
| | | | HAC | aligned/ | HAC | Approx. | | | | |
| Nanopore | Production | | fastq | filtered | frags lt | genomic | | | | |
| sample | site | type | frags | frags | 700 bp | coverage | — | — | — | — |
| HU002_10 | HU | Healthy | 2,399,362 | 1,812,081 | 1,799,882 | 0.139 | | | | |
| HU002_11 | HU | Healthy | 2,868,998 | 2,107,819 | 2,087,013 | 0.161 | | | | |
| HU002_12 | HU | Healthy | 1,868,764 | 1,411,697 | 1,377,698 | 0.106 | | | | |
| BC02 | ISPRO | Healthy | 3,026,018 | 2,451,660 | 2,389,691 | 0.185 | | | | |
| BC03 | ISPRO | Healthy | 3,231,074 | 2,640,869 | 2,592,758 | 0.2 | | | | |
| BC04 | ISPRO | Healthy | 3,738,131 | 3,030,606 | 2,976,871 | 0.23 | | | | |
| BC05 | ISPRO | Healthy | 6,570,342 | 5,428,254 | 5,229,034 | 0.404 | | | | |
| BC09 | ISPRO | Cancer | 3,083,998 | 2,525,079 | 2,482,299 | 0.192 | | | | |
| S1 | ISPRO | Cancer | 18,108,794 | 13,606,720 | 13,355,962 | 1.032 | | | | |
| BC08 | ISPRO | Cancer | 4,236,209 | 3,393,599 | 3,375,772 | 0.261 | | | | |
| BC01 | ISPRO | Cancer | 15,617,078 | 12,699,283 | 12,609,979 | 0.974 | | | | |
| BC10 | ISPRO | Cancer | 4,257,158 | 3,400,399 | 3,388,720 | 0.262 | | | | |
| BC11 | ISPRO | Cancer | 8,611,216 | 6,885,757 | 6,859,844 | 0.53 | | | | |
| Illumina sample | | | | | | | | | | |
| BC08 | ISPRO | Cancer | 22,325,079 | 15,627,068 | 15,627,068 | 1.207 | | | | |
| BC01 | ISPRO | Cancer | 17,259,281 | 12,328,923 | 12,328,923 | 0.952 | | | | |
| BC10 | ISPRO | Cancer | 22,846,324 | 15,459,002 | 15,459,002 | 1.194 | | | | |
| BC11 | ISPRO | Cancer | 23,960,716 | 17,275,596 | 17,275,596 | 1.334 | | | | |

| | | | 2022 | | | | | | | |
|---|---|---|---|---|---|---|---|---|---|---|
| | | | HAC | | 2019 | 2019 FAST | 2019 | | | |
| | | | Downsample | 2022 | FAST | aligned/ | FAST | | | |
| Nanopore | Production | | cfNano | HAC | fastq | filtered | frags lt | | | |
| sample | site | type | level | Downsample 2M | frags | frags | 700 bp | — | — | — |
| HU002_10 | HU | Healthy | | 1,799,882 | | | | | | |
| HU002_11 | HU | Healthy | | 2,000,000 | | | | | | |
| HU002_12 | HU | Healthy | | 1,377,698 | | | | | | |
| BC02 | ISPRO | Healthy | | 2,000,000 | 3,827,741 | 2,189,007 | 2,122,678 | | | |
| BC03 | ISPRO | Healthy | | 2,000,000 | 4,180,749 | 2,352,645 | 2,299,071 | | | |
| BC04 | ISPRO | Healthy | | 2,000,000 | 4,763,890 | 2,690,547 | 2,631,636 | | | |
| BC05 | ISPRO | Healthy | | 2,000,000 | 8,396,901 | 4,845,763 | 4,629,624 | | | |
| BC09 | ISPRO | Cancer | | 2,000,000 | 3,851,389 | 2,020,869 | 1,963,858 | | | |
| S1 | ISPRO | Cancer | | 2,000,000 | 18,956,437 | 10,192,974 | 9,785,417 | | | |
| BC08 | ISPRO | Cancer | | 2,000,000 | 5,285,817 | 2,631,108 | 2,600,897 | | | |
| BC01 | ISPRO | Cancer | | 2,000,000 | 20,190,561 | 11,211,981 | 11,091,313 | | | |
| BC10 | ISPRO | Cancer | | 2,000,000 | 5,266,247 | 2,627,621 | 2,602,835 | | | |
| BC11 | ISPRO | Cancer | | 2,000,000 | 10,851,450 | 5,335,161 | 5,278,324 | | | |
| Illumina sample | | | | | | | | | | |
| BC08 | ISPRO | Cancer | 3,375,772 | 2,000,000 | | | | | | |
| BC01 | ISPRO | Cancer | 12,328,923 | 2,000,000 | | | | | | |
| BC10 | ISPRO | Cancer | 3,388,720 | 2,000,000 | | | | | | |
| BC11 | ISPRO | Cancer | 6,859,844 | 2,000,000 | | | | | | |

| | | | | | Megaldon | Megalodon | DeepSignal | DeepSignal | | |
|---|---|---|---|---|---|---|---|---|---|---|
| | | | 2022 | 2022 HAC | normal | TCGA | normal | TCGA | | |
| Nanopore | Production | | HAC | ichorCNA | lung | LuAd | lung | LuAd | | |
| sample | site | type | ichorCNA | 2M | fraction | fraction | fraction | fraction | — | — |
| HU002_10 | HU | Healthy | 0 | 0 | 0.065 | 0.111 | 0.131 | 0.137 | | |
| HU002_11 | HU | Healthy | 0 | 0 | 0.017 | 0 | 0.032 | 0.022 | | |
| HU002_12 | HU | Healthy | 0 | 0 | 0 | 0.001 | 0.04 | 0.012 | | |
| BC02 | ISPRO | Healthy | 0 | 0 | 0 | 0 | 0 | 0 | | |
| BC03 | ISPRO | Healthy | 0 | 0 | 0.068 | 0.044 | 0.032 | 0.039 | | |
| BC04 | ISPRO | Healthy | 0 | 0 | 0 | 0.013 | 0.005 | 0 | | |
| BC05 | ISPRO | Healthy | 0 | 0 | 0 | 0 | 0 | 0 | | |
| BC09 | ISPRO | Cancer | 0.079 | 0.08 | 0.187 | 0.092 | 0.062 | 0.051 | | |
| S1 | ISPRO | Cancer | 0.238 | 0.238 | 0.306 | 0.211 | 0.264 | 0.204 | | |
| BC08 | ISPRO | Cancer | 0.149 | 0.131 | 0.185 | 0.133 | 0.078 | 0.098 | | |
| BC01 | ISPRO | Cancer | 0.252 | 0.25 | 0.244 | 0.219 | 0.198 | 0.221 | | |
| BC10 | ISPRO | Cancer | 0.271 | 0.33 | 0.619 | 0.438 | 0.538 | 0.421 | | |
| BC11 | ISPRO | Cancer | 0.367 | 0.288 | 0.402 | 0.274 | 0.377 | 0.256 | | |
| Illumina sample | | | | | | | | | | |
| BC08 | ISPRO | Cancer | 0.105 | 0.105 | | | | | | |
| BC01 | ISPRO | Cancer | 0.252 | 0.255 | | | | | | |
| BC10 | ISPRO | Cancer | 0.267 | 0.323 | | | | | | |
| BC11 | ISPRO | Cancer | 0.349 | 0.366 | | | | | | |

| | | | | | Megalodon | Megalodon | | | Normal | DeepSignal |
|---|---|---|---|---|---|---|---|---|---|---|
| | | | | | Normal | TCGA | | | lung | TCGA |
| | | | | | lung | LuAd | | DeepSignal | DeepSignal | LuAd |
| Nanopore | Production | | Megalodon | Megaldon | CpG | CpG | DeepSignal | 450k | CpG | CpG |
| sample | site | type | CpGs | 450k CpGs | count | count | CpGs | CpGs | count | count |
| HU002_10 | HU | Healthy | 4,219,443 | 70,312 | 330 | 1769 | 3,865,069 | 62,967 | 554 | 1623 |
| HU002_11 | HU | Healthy | 5,322,144 | 91,638 | 435 | 2280 | 4,928,546 | 82,975 | 755 | 2080 |
| HU002_12 | HU | Healthy | 3,892,137 | 66,157 | 347 | 1731 | 3,580,786 | 59,720 | 568 | 1626 |
| BC02 | ISPRO | Healthy | 5,615,377 | 93,321 | 424 | 2362 | 4,735,241 | 80,397 | 697 | 2095 |
| BC03 | ISPRO | Healthy | 5,817,793 | 94,821 | 471 | 2467 | 4,862,242 | 81,366 | 744 | 2185 |
| BC04 | ISPRO | Healthy | 6,600,725 | 109,412 | 495 | 2660 | 5,527,777 | 93,934 | 814 | 2353 |
| BC05 | ISPRO | Healthy | 13,091,82 | 211,188 | 927 | 4980 | 11,073,120 | 180,196 | 1590 | 4423 |
| BC09 | ISPRO | Cancer | 5,282,368 | 81,845 | 411 | 2109 | 4,316,207 | 69,276 | 634 | 1851 |
| S1 | ISPRO | Cancer | 20,314,67 | 399,302 | 1526 | 8290 | 18,877,704 | 305,471 | 2423 | 6870 |
| BC08 | ISPRO | Cancer | 5,866,622 | 92,236 | 426 | 2404 | 4,430,377 | 72,915 | 729 | 2008 |
| BC01 | ISPRO | Cancer | 24,786,69 | 321,739 | 1276 | 7076 | 17,048,495 | 274,377 | 2239 | 6363 |
| BC10 | ISPRO | Cancer | 6,034,817 | 95,136 | 425 | 2427 | 4,566,533 | 75,191 | 691 | 1956 |
| BC11 | ISPRO | Cancer | 10,734,74 | 158,244 | 729 | 4044 | 8,026,908 | 122,413 | 1144 | 3254 |
| Illumina sample | | | | | | | | | | |
| BC08 | ISPRO | Cancer | | | | | | | | |
| BC01 | ISPRO | Cancer | | | | | | | | |
| BC10 | ISPRO | Cancer | | | | | | | | |
| BC11 | ISPRO | Cancer | | | | | | | | |

TABLE 2
Whole-genome bisulfite sequencing (WGBS) datasets used as controls for
methylation analysis.

| | | | | | short |
|---|---|---|---|---|---|
| Study | Study DOI | ID | database | original name | name |
| Fox-Fisher et al., | 10.7554/eLife.70520 | GSM5663195 | GEO | CNVS-NORM- | FF1 |
| eLife 2021 | | | | 110000263-cfDNA- | |
| | | | | WGBS-Rep1 | |
| Fox-Fisher et al., | 10.7554/eLife.70520 | GSM5663196 | GEO | CNVS-NORM- | FF21 |
| eLife 2021 | | | | 110000264-cfDNA- | |
| | | | | WGBS-Rep1 | |
| Fox-Fisher et al., | 10.7554/eLife.70520 | GSM5663197 | GEO | CNVS-NORM- | FF17 |
| eLife 2021 | | | | 110027051-cfDNA- | |
| | | | | WGBS-Rep1 | |
| Fox-Fisher et al., | 10.7554/eLife.70520 | GSM5663198 | GEO | CNVS-NORM- | FF20 |
| eLife 2021 | | | | 110027054-cfDNA- | |
| | | | | WGBS-Rep2 | |
| Fox-Fisher et al., | 10.7554/eLife.70520 | GSM5663199 | GEO | CNVS-NORM- | FF14 |
| eLife 2021 | | | | 110029639-cfDNA- | |
| | | | | WGBS-Rep1 | |
| Fox-Fisher et al., | 10.7554/eLife.70520 | GSM5663200 | GEO | CNVS-NORM- | FF19 |
| eLife 2021 | | | | 110029979-cfDNA- | |
| | | | | WGBS-Rep1 | |
| Fox-Fisher et al., | 10.7554/eLife.70520 | GSM5663201 | GEO | CNVS-NORM- | FF18 |
| eLife 2021 | | | | 110032739-cfDNA- | |
| | | | | WGBS-Rep1 | |
| Fox-Fisher et al., | 10.7554/eLife.70520 | GSM5663202 | GEO | CNVS-NORM- | FF2 |
| eLife 2021 | | | | 110032789-cfDNA- | |
| | | | | WGBS-Rep1 | |
| Fox-Fisher et al., | 10.7554/eLife.70520 | GSM5663203 | GEO | CNVS-NORM- | FF3 |
| eLife 2021 | | | | 110033253-cfDNA- | |
| | | | | WGBS-Rep1 | |
| Fox-Fisher et al., | 10.7554/eLife.70520 | GSM5663204 | GEO | CNVS-NORM- | FF4 |
| eLife 2021 | | | | 110033397-cfDNA- | |
| | | | | WGBS-Rep1 | |
| Fox-Fisher et al., | 10.7554/eLife.70520 | GSM5663205 | GEO | CNVS-NORM- | FF5 |
| eLife 2021 | | | | 110033577-cfDNA- | |
| | | | | WGBS-Rep1 | |
| Fox-Fisher et al., | 10.7554/eLife.70520 | GSM5663206 | GEO | CNVS-NORM- | FF16 |
| eLife 2021 | | | | 110033633-cfDNA- | |
| | | | | WGBS-Rep1 | |
| Fox-Fisher et al., | 10.7554/eLife.70520 | GSM5663207 | GEO | CNVS-NORM- | FF6 |
| eLife 2021 | | | | 110033727-cfDNA- | |
| | | | | WGBS-Rep1 | |
| Fox-Fisher et al., | 10.7554/eLife.70520 | GSM5663208 | GEO | CNVS-NORM- | FF15 |
| eLife 2021 | | | | 110033745-cfDNA- | |
| | | | | WGBS-Rep1 | |
| Fox-Fisher et al., | 10.7554/eLife.70520 | GSM5663209 | GEO | CNVS-NORM- | FF7 |
| eLife 2021 | | | | 110033746-cfDNA- | |
| | | | | WGBS-Rep1 | |
| Fox-Fisher et al., | 10.7554/eLife.70520 | GSM5663210 | GEO | CNVS-NORM- | FF8 |
| eLife 2021 | | | | 110033750-cfDNA- | |
| | | | | WGBS-Rep1 | |
| Fox-Fisher et al., | 10.7554/eLife.70520 | GSM5663211 | GEO | CNVS-NORM- | FF22 |
| eLife 2021 | | | | 110033907-cfDNA- | |
| | | | | WGBS-Rep1 | |
| Fox-Fisher et al., | 10.7554/eLife.70520 | GSM5663212 | GEO | CNVS-NORM- | FF11 |
| eLife 2021 | | | | 110033912-cfDNA- | |
| | | | | WGBS-Rep1 | |
| Fox-Fisher et al., | 10.7554/eLife.70520 | GSM5663213 | GEO | CNVS-NORM- | FF9 |
| eLife 2021 | | | | 110033933-cfDNA- | |
| | | | | WGBS-Rep1 | |
| Fox-Fisher et al., | 10.7554/eLife.70520 | GSM5663214 | GEO | CNVS-NORM- | FF12 |
| eLife 2021 | | | | 110036059-cfDNA- | |
| | | | | WGBS-Rep1 | |
| Fox-Fisher et al., | 10.7554/eLife.70520 | GSM5663215 | GEO | CNVS-NORM- | FF13 |
| eLife 2021 | | | | 110036107-cfDNA- | |
| | | | | WGBS-Rep1 | |
| Fox-Fisher et al., | 10.7554/eLife.70520 | GSM5663216 | GEO | CNVS-NORM- | FF23 |
| eLife 2021 | | | | 110036138-cfDNA- | |
| | | | | WGBS-Rep1 | |
| Fox-Fisher et al., | 10.7554/eLife.70520 | GSM5663217 | GEO | CNVS-NORM- | FF10 |
| eLife 2021 | | | | 110036152-cfDNA- | |
| | | | | WGBS-Rep1 | |
| Nguyen et al., Sci. | 10.1038/s41598-021- | 10.6084/m9.figsh | Figshare | LBHC04 | N01 |
| Reports 2021 | 95985-6 | are.16817941.v1 | | | |
| Nguyen et al., Sci. | 10.1038/s41598-021- | 10.6084/m9.figsh | Figshare | LBHC07 | N02 |
| Reports 2021 | 95985-6 | are.16817941.v1 | | | |
| Nguyen et al., Sci. | 10.1038/s41598-021- | 10.6084/m9.figsh | Figshare | LBHC10 | N03 |
| Reports 2021 | 95985-6 | are.16817941.v1 | | | |
| Nguyen et al., Sci. | 10.1038/s41598-021- | 10.6084/m9.figsh | Figshare | L11133 | N04 |
| Reports 2021 | 95985-6 | are.16817941.v1 | | | |
| Nguyen et al., Sci. | 10.1038/s41598-021- | 10.6084/m9.figsh | Figshare | NA044 | N05 |
| Reports 2021 | 95985-6 | are.16817941.v1 | | | |
| Nguyen et al., Sci. | 10.1038/s41598-021- | 10.6084/m9.figsh | Figshare | NA011 | N06 |
| Reports 2021 | 95985-6 | are.16817941.v1 | | | |
| Nguyen et al., Sci. | 10.1038/s41598-021- | 10.6084/m9.figsh | Figshare | NA016 | N07 |
| Reports 2021 | 95985-6 | are.16817941.v1 | | | |
| Nguyen et al., Sci. | 10.1038/s41598-021- | 10.6084/m9.figsh | Figshare | NA045 | N08 |
| Reports 2021 | 95985-6 | are.16817941.v1 | | | |
| Nguyen et al., Sci. | 10.1038/s41598-021- | 10.6084/m9.figsh | Figshare | NA012 | N09 |
| Reports 2021 | 95985-6 | are.16817941.v1 | | | |
| Nguyen et al., Sci. | 10.1038/s41598-021- | 10.6084/m9.figsh | Figshare | NA035 | N10 |
| Reports 2021 | 95985-6 | are.16817941.v1 | | | |
| Nguyen et al., Sci. | 10.1038/s41598-021- | 10.6084/m9.figsh | Figshare | L10382 | N11 |
| Reports 2021 | 95985-6 | are.16817941.v1 | | | |
| Nguyen et al., Sci. | 10.1038/s41598-021- | 10.6084/m9.figsh | Figshare | L10900 | N12 |
| Reports 2021 | 95985-6 | are.16817941.v1 | | | |
| Nguyen et al., Sci. | 10.1038/s41598-021- | 10.6084/m9.figsh | Figshare | L10814 | N13 |
| Reports 2021 | 95985-6 | are.16817941.v1 | | | |
| Nguyen et al., Sci. | 10.1038/s41598-021- | 10.6084/m9.figsh | Figshare | L10695 | N14 |
| Reports 2021 | 95985-6 | are.16817941.v1 | | | |
| Nguyen et al., Sci. | 10.1038/s41598-021- | 10.6084/m9.figsh | Figshare | L10821 | N15 |
| Reports 2021 | 95985-6 | are.16817941.v1 | | | |
| Nguyen et al., Sci. | 10.1038/s41598-021- | 10.6084/m9.figsh | Figshare | L10046 | N16 |
| Reports 2021 | 95985-6 | are.16817941.v1 | | | |
| Nguyen et al., Sci. | 10.1038/s41598-021- | 10.6084/m9.figsh | Figshare | L10381 | N17 |
| Reports 2021 | 95985-6 | are.16817941.v1 | | | |
| Nguyen et al., Sci. | 10.1038/s41598-021- | 10.6084/m9.figsh | Figshare | L10882 | N18 |
| Reports 2021 | 95985-6 | are.16817941.v1 | | | |
| Nguyen et al., Sci. | 10.1038/s41598-021- | 10.6084/m9.figsh | Figshare | L10672 | N19 |
| Reports 2021 | 95985-6 | are.16817941.v1 | | | |
| Nguyen et al., Sci. | 10.1038/s41598-021- | 10.6084/m9.figsh | Figshare | L11034 | N20 |
| Reports 2021 | 95985-6 | are.16817941.v1 | | | |
| Nguyen et al., Sci. | 10.1038/s41598-021- | 10.6084/m9.figsh | Figshare | L10955 | N21 |
| Reports 2021 | 95985-6 | are.16817941.v1 | | | |
| Sun et al., PNAS | 10.1073/pnas.1508736112 | EGAD00001001602 | EGA | CTR101 | NA |
| 2015 | | | | | |
| Sun et al., PNAS | 10.1073/pnas.1508736112 | EGAD00001001602 | EGA | CTR103 | NA |
| 2015 | | | | | |
| Sun et al., PNAS | 10.1073/pnas.1508736112 | EGAD00001001602 | EGA | CTR104 | NA |
| 2015 | | | | | |
| Sun et al., PNAS | 10.1073/pnas.1508736112 | EGAD00001001602 | EGA | CTR106 | NA |
| 2015 | | | | | |
| Sun et al., PNAS | 10.1073/pnas.1508736112 | EGAD00001001602 | EGA | CTR107 | NA |
| 2015 | | | | | |
| Sun et al., PNAS | 10.1073/pnas.1508736112 | EGAD00001001602 | EGA | CTR108 | NA |
| 2015 | | | | | |
| Sun et al., PNAS | 10.1073/pnas.1508736112 | EGAD00001001602 | EGA | CTR110 | NA |
| 2015 | | | | | |
| Sun et al., PNAS | 10.1073/pnas.1508736112 | EGAD00001001602 | EGA | CTR111 | NA |
| 2015 | | | | | |
| Sun et al., PNAS | 10.1073/pnas.1508736112 | EGAD00001001602 | EGA | CTR113 | NA |
| 2015 | | | | | |
| Sun et al., PNAS | 10.1073/pnas.1508736112 | EGAD00001001602 | JEGA | CTR114 | NA |
| 2015 | | | | | |
| Sun et al., PNAS | 10.1073/pnas.1508736112 | EGAD00001001602 | EGA | CTR117 | NA |
| 2015 | | | | | |
| Sun et al., PNAS | 10.1073/pnas.1508736112 | EGAD00001001602 | JEGA | CTR118 | NA |
| 2015 | | | | | |
| Sun et al., PNAS | 10.1073/pnas.1508736112 | EGAD00001001602 | EGA | CTR126 | NA |
| 2015 | | | | | |
| Sun et al., PNAS | 10.1073/pnas.1508736112 | EGAD00001001602 | EGA | CTR127 | NA |
| 2015 | | | | | |
| Sun et al., PNAS | 10.1073/pnas.1508736112 | EGAD00001001602 | EGA | CTR128 | NA |
| 2015 | | | | | |
| Sun et al., PNAS | 10.1073/pnas.1508736112 | EGAD00001001602 | EGA | CTR129 | NA |
| 2015 | | | | | |
| Sun et al., PNAS | 10.1073/pnas.1508736112 | EGAD00001001602 | EGA | CTR131 | NA |
| 2015 | | | | | |
| Sun et al., PNAS | 10.1073/pnas.1508736112 | EGAD00001001602 | EGA | CTR132 | NA |
| 2015 | | | | | |
| Sun et al., PNAS | 10.1073/pnas.1508736112 | EGAD00001001602 | EGA | CTR134 | NA |
| 2015 | | | | | |
| Sun et al., PNAS | 10.1073/pnas.1508736112 | EGAD00001001602 | EGA | CTR147 | NA |
| 2015 | | | | | |
| Sun et al., PNAS | 10.1073/pnas.1508736112 | EGAD00001001602 | EGA | CTR148 | NA |
| 2015 | | | | | |
| Sun et al., PNAS | 10.1073/pnas.1508736112 | EGAD00001001602 | EGA | CTR149 | NA |
| 2015 | | | | | |
| Sun et al., PNAS | 10.1073/pnas.1508736112 | EGAD00001001602 | JEGA | CTR150 | NA |
| 2015 | | | | | |
| Sun et al., PNAS | 10.1073/pnas.1508736112 | EGAD00001001602 | EGA | CTR151 | NA |
| 2015 | | | | | |
| Sun et al., PNAS | 10.1073/pnas.1508736112 | EGAD00001001602 | EGA | CTR152 | NA |
| 2015 | | | | | |
| Sun et al., PNAS | 10.1073/pnas.1508736112 | JEGAD00001001602 | EGA | CTR153 | NA |
| 2015 | | | | | |
| Sun et al., PNAS | 10.1073/pnas.1508736112 | EGAD00001001602 | JEGA | CTR154 | NA |
| 2015 | | | | | |
| Sun et al., PNAS | 10.1073/pnas.1508736112 | EGAD00001001602 | EGA | CTR84 | NA |
| 2015 | | | | | |
| Sun et al., PNAS | 10.1073/pnas.1508736112 | EGAD00001001602 | EGA | CTR85 | NA |
| 2015 | | | | | |
| Sun et al., PNAS | 10.1073/pnas.1508736112 | EGAD00001001602 | EGA | CTR86 | NA |
| 2015 | | | | | |
| Sun et al., PNAS | 10.1073/pnas.1508736112 | EGAD00001001602 | EGA | CTR97 | NA |
| 2015 | | | | | |
| Sun et al., PNAS | 10.1073/pnas.1508736112 | EGAD00001001602 | EGA | CTR98 | NA |
| 2015 | | | | | |

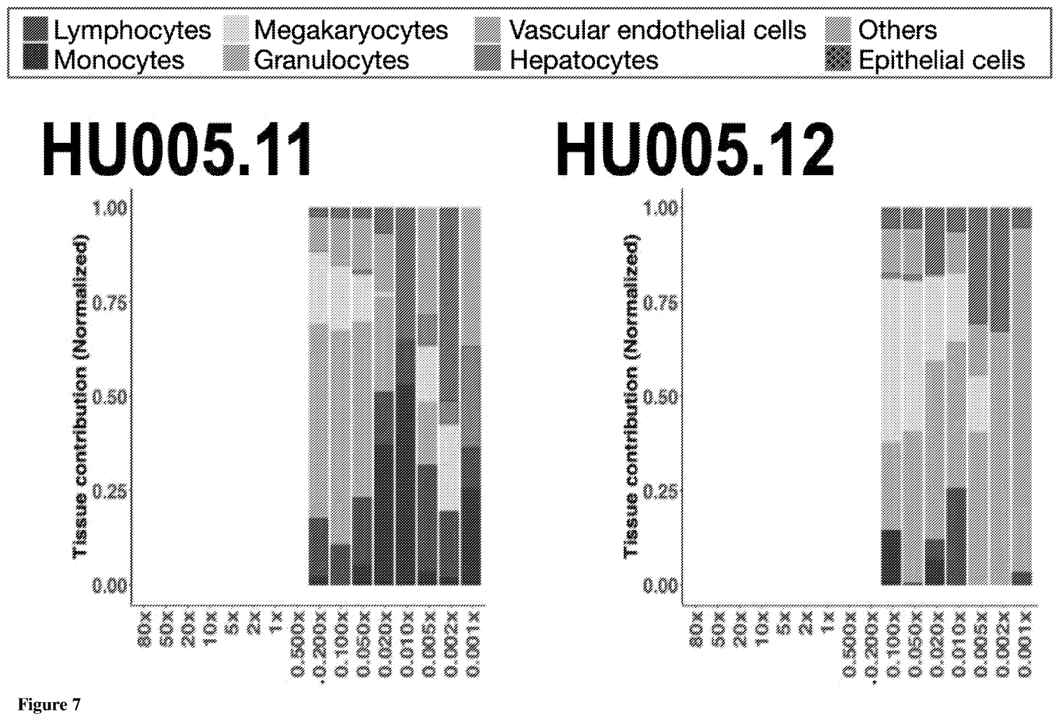

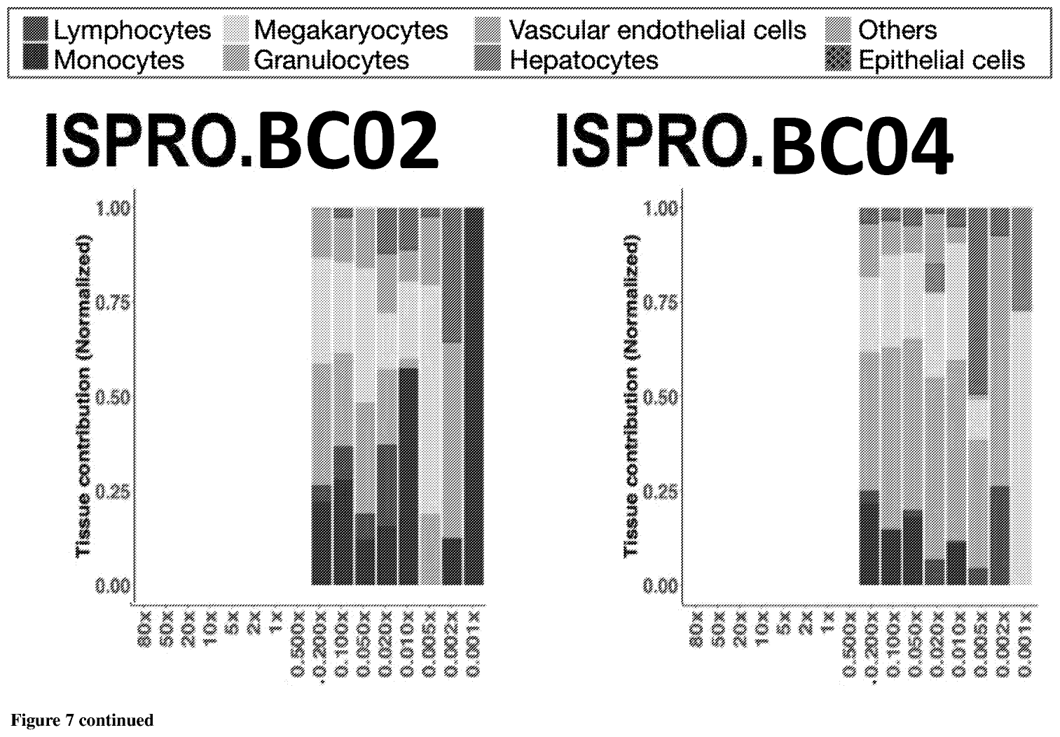

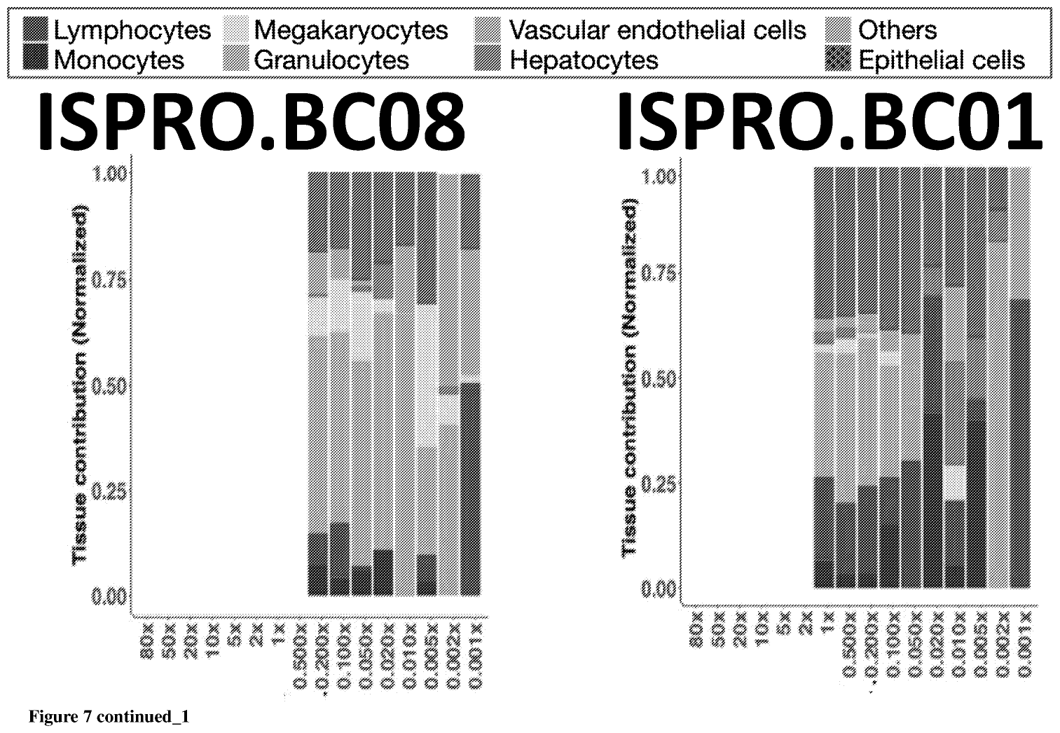

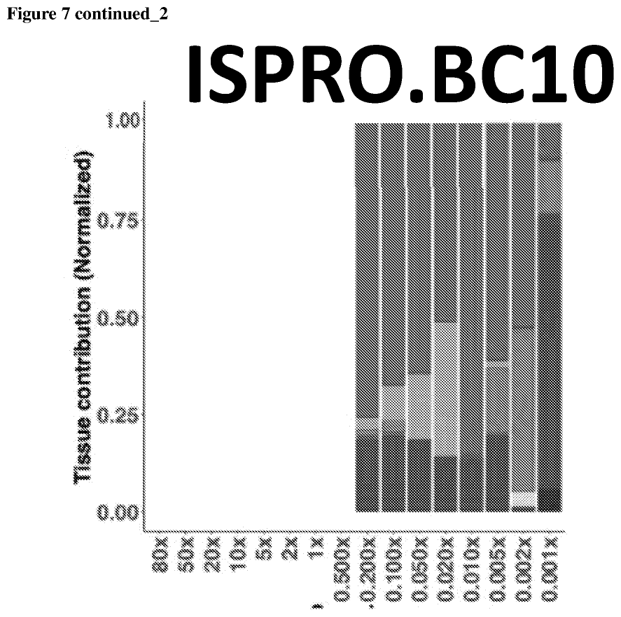

The healthy cfNano individuals were divided into two groups based on source site, with one being collected and sequenced in Italy (“BC” samples) and one in Israel (“HU” samples). Despite the HU samples being lower coverage (two were between 0.10-0.15× depth), they displayed relatively similar cell type proportions (FIG. 1B-1C and FIG. 7).

In addition to healthy individuals, the Nguyen WGBS dataset and our cfNano dataset also contained individuals being treated for lung adenocarcinoma, marked as “LuAd” in FIG. 1B-1C. In the Nguyen WGBS study, samples were collected at the time of acquired resistance to EGFR-inhibitors, and were divided into those that acquired resistance mutations in EGFR itself (labeled “on” for on-target) vs. those that acquired amplifications in alternative oncogenes MET/ERBB2 (labeled “off” for off-target). The epithelial cell fraction was much higher in the on-target patients, while the off-target patients had very low or no epithelial fraction (FIG. 1B), consistent with the absence of CNAs in the off-target samples in the original study. The 6 LuAd samples in our cfNano study had similarly high epithelial fraction (FIG. 1B), which was significantly higher than in the healthy patients (p=0.004). In all WGBS and cfNano samples, full depth results were highly similar to 0.2× downsampled results (FIG. 1C and FIGS. 5-7). Interestingly, while the Nguyen et al. study interpreted the normal-like methylation levels of the “off-target” tumors as a difference in cancer methylation patterns, our deconvolution results strongly suggest that it is due to the relative amount of cancer DNA circulating in the blood.

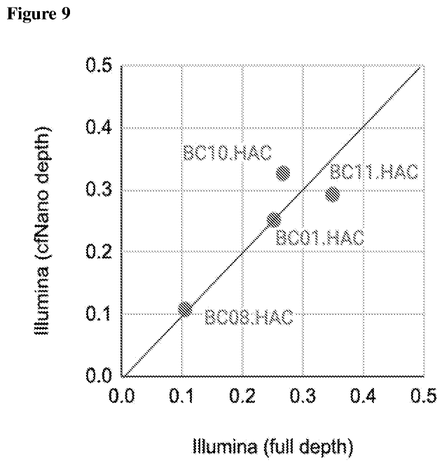

The fraction of cancer cells in cfDNA (“tumor fraction”) can be estimated from somatic copy number alterations (CNAs) using the ichorCNA tool, for cancer cells that contain a sufficient degree of aneuploidy. We estimated tumor fraction for our cfNano samples and four matched Illumina WGS samples from LuAd patients (FIG. 1D, Table 1). While the Illumina samples were sequenced at significantly higher depth (median 1.3×), the tumor fraction estimates were highly similar between cfNano and Illumina sequencing (FIG. 1E). Interestingly, the ichorCNA tumor fractions were more similar to the high-depth Illumina samples than the Illumina samples themselves were when downsampled to the same depth as the cfNano samples (FIG. 9A).

To compare ichorCNA tumor fraction estimates to methylation-based estimates, we designed a “two-component” deconvolution method based on NNLS regression that used 2,253 CpGs with differential methylation between sorted lung epithelia and healthy plasma. This was based on the same array-based MethAtlas samples as the full deconvolution (FIG. 1F). 330-1,526 of these CpGs were covered by each cfNano sample, which were the CpGs used for NNLS deconvolution. These DNA methylation based estimates of lung fraction and the ichorCNA estimates of cancer cell fraction were largely in agreement (FIG. 1F, bottom), with all the six LuAd samples having significantly higher lung fraction compared to the seven healthy plasma samples (p=0.003). Two LuAd cases were markedly higher in the methylation-based than the CNA-based estimate (BC09 and BC10). While we have no independent data to determine which was the more accurate estimate, we hypothesize that the discrepancy may be due to either whole-genome doubling (WGD) events that are not detected by ichorCNA (WGD occurs in 297/503 or 59% of LuAd tumors from the TCGA project), or damage to normal lung cells surrounding the tumor which die and shed their DNA into circulation.

To verify the robustness of methylation-based deconvolution, we used a mutually exclusive set of 13,770 CpGs that could distinguish TCGA LuAd tumors from healthy plasma, but were not found in the normal lung epithelia set (FIG. 1H). Before applying the NNLS regression, since most TCGA LuAd samples contain a significant fraction of leukocytes, we corrected the methylation levels of the TCGA LuAd samples based on their non-cancer cell contamination (“purity correction”). After this correction, the tumor fraction estimates of our cfNano samples were highly similar to those based on normal-lung specific CpGs (FIG. 1H, bottom), despite the fact that the two CpG sets were completely non-overlapping. One HU healthy sample (HU005.10) had a higher lung fraction estimate than one of the cancer samples, possibly because this was the cfNano sample with the lowest sequencing coverage (0.11×). However, the methylation-based tumor fraction was still significantly higher in the LuAd samples than healthy controls (p=0.004).

We performed all deconvolution analysis using a second, and older, base modification caller (DeepSignal, Peng Ni et al., “DeepSignal: Detecting DNA Methylation State from Nanopore Sequencing Reads Using Deep-Learning”, Bioinformatics 35, no. 22 (2019), hereby incorporated by reference in its entirety). While Megalodon called 10-20% more CpGs, the majority of CpGs called were in common between the two methods and had identical methylation states (FIG. 10A). Both the full cell-type deconvolution (FIG. 10B) and the two-component deconvolution (FIG. 10C) were highly similar between the two callers.

### Example 2: Genomic Context of DNA Methylation Changes Detected Using cfNano

The deconvolution analysis above was based on unannotated differentially methylated regions. In order to investigate the genomic context of lung cancer specific DNA methylation, we analyzed one hypomethylation feature associated with cell of origin (lineage-specific transcription factor binding sites) and one associated specifically with transformation (global hypomethylation). For the TFBS analysis, we identified 5,974 predicted TFBS that were specific to lung epithelia based on a single-cell ATAC-seq atlas of open chromatin within lung and other primary human tissues from Zhang et al., In that study, adult lung tissues from multiple donors contained a strong cluster of lung pneumocyte-specific open chromatin regions (“Pal” cluster). This cluster was most strongly enriched for the binding motif for the transcription factor NKX2-1, which is a master regulatory transcription factor in this cell type. NKX2-1 activity is also known to be highly restricted to this cell type, and NKX2-1 binding sites were also the most enriched within lung adenocarcinoma ATAC-seq sites in an independent study (M. Ryan Corces et al., “The Chromatin Accessibility Landscape of Primary Human Cancers”, Science 362, no. 6413 (2018), hereby incorporated by reference in its entirety). Because open chromatin regions are almost universally hypomethylated, we hypothesized that the 5,974 predicted NKX2-1 TFBS in lung pneumocytes would be specifically hypomethylated in healthy lung tissues and in lung tumors. We confirmed this using WGBS data from TCGA by Zhou et al. (FIG. 11A).

We next plotted plasma cfDNA methylation levels at these same predicted NKX2-1 sites from the published Illumina WGBS studies and our cfNano study (FIG. 2A). In healthy samples from three WGBS studies and our own cfNano samples, NKX2-1 sites were fully methylated. In contrast, the LuAd samples from both the Nguyen et al. WGBS study (FIG. 2A, middle) and our cfNano study (FIG. 2A, right) had substantial demethylation. In both studies demethylation could only be observed in the higher tumor fraction samples (“on-target” samples in the WGBS study, and samples with ichorCNA TF>0.15 in the cfNano study). As a negative control, we selected predicted TFBS from a cell type not expected to be found either in healthy plasma or LuAd. We used the adrenal cortical cluster (“Adc” cluster) from Zhang et al., which was highly enriched for the KLF5 binding motif. These sites were fully methylated in plasma samples from both healthy and LuAd individuals (FIG. 11B). cfNano profiles were nearly identical using DeepSignal methylation calling (FIG. 11C).

Global DNA hypomethylation is one of the hallmarks of the cancer epigenome. It has long been proposed as a general marker for circulating tumor DNA, and this was recently verified for lung cancer using shallow plasma cfDNA WGBS. We have shown that this “global” hypomethylation is not completely global, and occurs preferentially within large domains called Partially Methylated Domains (PMDs) and specifically at CpGs with a local sequence context termed “solo-WCGW”. We replotted WGBS methylation data from TCGA normal lung and lung tumor tissues, showing a typical chromosome arm where strong hypomethylation occurs within the PMD regions identified in Zhou et al. in the cancer samples (FIG. 2B, top). In our cfNano samples, strong hypomethylation was also found exclusively in the cancer samples in the same PMD regions (FIG. 2B, bottom). To quantify this genome-wide, we plotted the methylation change (relative to the sample-specific whole-genome average) of PMD solo-WCGW CpGs within each 10 Mbp genomic bin that overlapped a common PMD region from Zhou et la. (FIG. 2C). As expected, five of the six cancer samples were significantly hypomethylated relative to the healthy controls (p<0.0001). Overall, there was significantly more hypomethylation across all cancer sample bins (mean=−0.10.SD=0.07) than across all healthy sample bins (mean=−0.05,SD=0.04), corresponding to a p-value<2.2e-16 by one-sided Wilcoxon test. In the final LuAd case (BC11), no PMD hypomethylation could be detected (FIG. 2C). This is not surprising given the high variability associated with global hypomethylation in cancer, a process that is not entirely understood but is affected both passively, through mitotic divisions, and actively, by dysregulation of several chromatin modifiers.

Reasoning that copy number altered regions would have skewed proportions of tumor-derived DNA and thus different levels of PMD hypomethylation, we divided the PMD bins based on the copy number status of each sample from ichorCNA. In four of the five cases with significant hypomethylation overall, the amplified bins had significantly more hypomethylation than diploid regions (FIG. 2D). In the one remaining case (BC09), there were not enough PMDs with CNAs for an accurate measurement. Conversely, deleted showed significantly less hypomethylation than diploid regions, although this trend only reached statistical significance in two cases. In the future, the combined analysis of CNAs and global hypomethylation may provide a stronger cancer-specific signal than each feature alone. PMD hypomethylation profiles were nearly identical using DeepSignal methylation calling (FIG. 11D-11F).

### Example 3: cfNano Preserves Nucleosome Positioning Signal

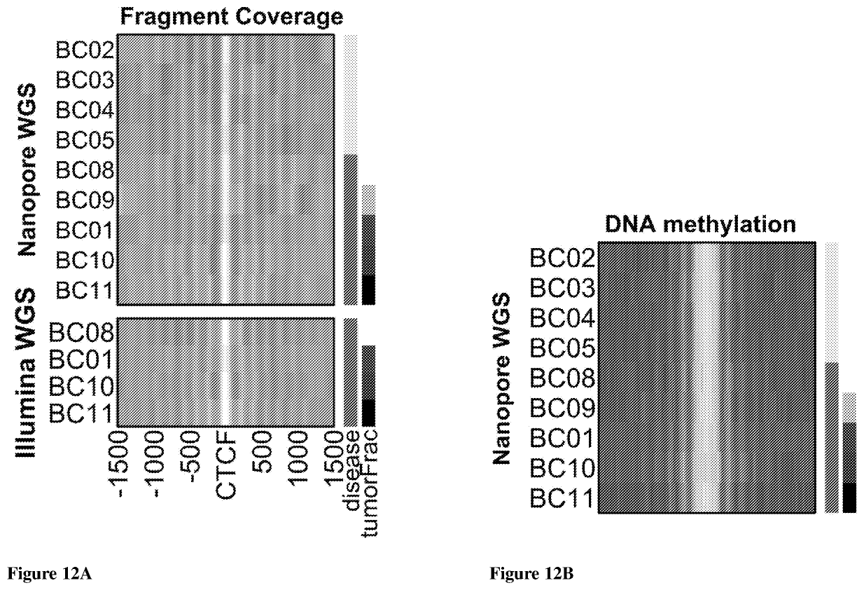

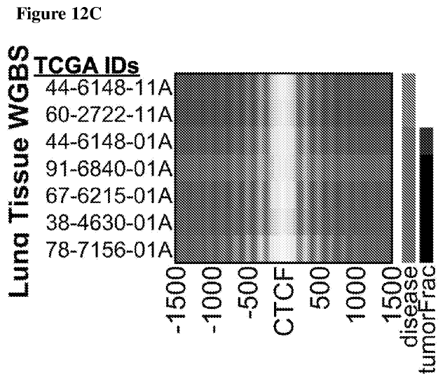

Cell-free DNA circulates primarily as mononucleosomal fragments, and mapping the positions of these mononucleosomes can be used to identify cell-type specific. CTCF binding sites provide a good test of whether these signals are detectable, since they eject a central nucleosome and position 10 phased nucleosomes on either side of their binding site (FIG. 3A). Around a set of 9,780 CTCF binding sites, cfNano mononucleosome locations recapitulated this expected pattern (FIG. 3B, top), which was identical to the pattern based on matched Illumina WGS of greater sequence depth (FIG. 3B, bottom). These were also identical when both cfNano and Illumina libraries were downsampled to an equal number of 2M fragments (FIG. 3C). It has been reported that the CTCF binding site also demethylates CpGs located approximately 200 bp on either side, and this DNA methylation pattern was also recapitulated in our cfNano samples (FIG. 12).

We tried the same mononucleosome mapping approach for the 5,974 lung-specific NKX2-1 TFBS from FIG. 2A. While the demethylation signal was detectable (FIG. 2A), we could not detect any mononucleosome positioning signal (data not shown). Lung-specific nucleosome positions would only be present on a fraction of the fragments, so the signal from these fragments may be masked by those from non-lung cell types. But given that the inherent nucleosome positioning information is present (as shown by the CTCF example), more advanced normalization and quantification techniques may reveal these cell-type specific fragments more sensitively in the future.

### Example 4: Cancer-Associated Fragmentation Length Features of cfNano Vs. Illumina WGS

Specific fragment lengths have been associated with cancer-derived cfDNA fragments and these have been used as accurate cancer classifiers. Specifically, shorter mononucleosome fragments (<150 bp) tend to be enriched for cancer-derived fragments. Density plots of fragment length showed that our cfNano cancer samples were enriched in these short mononucleosome fragments relative to healthy controls (FIG. 4A). We used the definition from Mouliere et al., and Cristiano et al. to calculate the ratio of short mononucleosomes (100-150 bp) to all mononucleosomes (100-220 bp). The short mononucleosome ratio was significantly higher in the high tumor fraction cancer cases (mean=0.24. SD-0.03) than in the healthy cases (mean=0.16. SD-0.03), which corresponded to a t-test p-value of p=0.038 (FIG. 4B). We compared these ratios calculated from our cfNano libraries with those calculated from the matched Illumina WGS libraries which were available for four of the six cancer samples, and the two library types were strongly correlated (FIG. 4C). We hypothesized that improvements to Nanopore basecalling could improve alignment and adapter (61 bp barcode) trimming, so we also compared base calling done with the real-time Guppy basecaller at the time of sequencing (“2019” version) to the new “high accuracy calling” basecalling (“HAC”) performed on all samples in 2022. The new ratios with the new basecalling were slightly more similar to the matched Illumina libraries (FIG. 4C).

While they have not been studied as extensively as mononucleosomes, it has been shown that dinucleosomes were significantly shorter in cancer fragments than non-cancer fragments. This is clear from the density plots of our cfNano samples (FIG. 4A), so we used the size range suggested by ref. Mouliere et al., “Enhanced Detection of Circulating Tumor DNA by Fragment Size Analysis”. to calculate the ratio between short dinucleosomes (275-325 bp) and all dinucleosomes (275-400 bp). The short dinucleosome ratio showed even more separation between cancer vs. healthy cfNano samples than the mononucleosome ratio, with the high tumor fraction cancer cases (mean=0.62, SD=0.01) than in the healthy cases (mean=0.30, SD=0.04), corresponding to a t-test p-value of p=2E-7 (FIG. 4D). We compared dinucleosome ratios calculated from our cfNano libraries with those calculated from the matched Illumina WGS libraries, and they were nearly perfectly correlated (FIG. 4E). When we looked across all samples, the short mononucleosome ratio was highly correlated with the short dinucleosome ratio (FIG. 13D). Interestingly, this correlation held across the healthy samples as well as the cancer samples, indicating that the same underlying mechanism affects circulating cfDNA from both cancer and non-cancer cell types.

One of our cfNano samples used a different (non-barcoded) adapter design method from all other libraries, and this sample was a clear outlier in fragment length (FIG. 13A-13D). This reinforces the caution that should be taken when comparing fragmentomic features across different library designs. We also investigated the effect on sequencing depth on cancer-associated features, by comparing full-depth datasets with datasets created by randomly choosing 2M fragments for each library (FIG. 13E-13H). Sequence depth had almost no effect on either cfNano or Illumina samples down to 2M fragments.

### Example 5: Cancer-Associated Fragment End Features of cfNano Vs. Illumina WGS

The four bases immediately flanking cfDNA fragmentation sites have a biased sequence composition that differs between cancer-derived and non-cancer-derived fragments. To study this in our cfNano samples, we first plotted the 25 most abundant 4-mer end motifs that were identified in an Illumina-based study of healthy plasma cfDNA (R. W. Y. Chan et al.) using a heatmap to indicate motif frequencies in each of our cfNano and matched Illumina samples (FIG. 4F). There was broad agreement across all samples, although some differences between cfNano and Illumina libraries were clearly noticeable. When we plotted average Nanopore vs. Illumina frequencies for all 256 possible 4-mers, it appeared that the less abundant motifs had slightly higher frequencies in Nanopore, while the more abundant motifs had slightly lower frequencies in Nanopore (FIG. 4G). Nevertheless, the relative frequencies were highly concordant overall (PCC=0.97). These were slightly more concordant when we used the 2022 “high accuracy” (HAC) basecalling compared to the original 2019 basecalling (PCC=0.97 vs. PCC=0.96). The degree of difference between the two batches of cfNano healthy samples (“BC” sequenced in 2019 and “HU” sequenced in 2022) showed only slight differences using the HAC basecalling (PCC=0.99).

Of particular interest is the CCCA end motif, which is typically the most abundant 4-mer in healthy plasma and its reduction was shown to be a cancer marker in several cancer types, including lung cancer. CCCA indeed has the highest frequency across all our cfNano and Illumina WGS samples (FIG. 4F-4H), and was significantly lower in our three high tumor fraction cancer samples than the healthy samples (FIG. 4I). However, there was a clear difference between the healthy samples generated in the “HU” and “ISPRO” batches, which we presume to be technical since these two batches behaved similarly with respect to fragment length and methylation features. We therefore only did a direct statistical comparison within the ISPRO batch, and indeed CCCA frequency in high TF tumors (mean=1.6,SD=0.06) was significantly lower than in ISPRO healthy samples (mean=1.9.SD=0.13), leading to a t-test p-value=0.007 (FIG. 4I).

We found additional 16 motifs that were as significant as CCCA, although none survived FDR adjustment and so will have to be validated in larger studies (Table 3). Like fragment lengths, end motif frequencies were not sensitive to downsampling to 2M fragments (FIG. 14A-D). Additionally, the relative frequencies of four cfNano cancer samples were not concordant with their matched Illumina WGS libraries (FIG. 4J). We conclude from this that end motifs are particularly sensitive to changes in library strategy and sequencing platform, and caution must be taken when comparing across multiple batches. This is not surprising, given that fragment representation can be skewed by a number of variables during library construction and amplification, as well as sequencing errors and downstream bioinformatic steps such as adapter trimming (in our cfNano processing, we also exclude fragment ends that are soft-clipped). End motifs are highly susceptible to these biases, because even a single base pair difference results in a completely different motif. Recent benchmarking has highlighted how error frequencies can differ by sequence context between the Nanopore and Illumina platforms.

TABLE 3
Tumor vs. normal differences for 4-mer end motifs

| | | | P- | FDR |
|---|---|---|---|---|
| | Mean | Mean | value | adjusted |
| | ISPRO | HIGH | HIGH | p-value |
| Motif | healthy | TF | TF | HIGH TF |
| TTCC | 0.14 | 0.179 | 0 | 0.074 |
| TTCT | 0.235 | 0.299 | 0.001 | 0.074 |
| TTCA | 0.202 | 0.262 | 0.001 | 0.083 |
| ATAG | 0.179 | 0.252 | 0.002 | 0.083 |
| AAGC | 0.177 | 0.204 | 0.002 | 0.083 |
| TTCG | 0.01 | 0.012 | 0.002 | 0.083 |
| AAAG | 0.601 | 0.642 | 0.003 | 0.083 |
| GGCT | 0.663 | 0.593 | 0.003 | 0.083 |
| GCCG | 0.074 | 0.061 | 0.003 | 0.083 |
| ACCG | 0.062 | 0.05 | 0.004 | 0.083 |
| CTGT | 0.438 | 0.512 | 0.004 | 0.083 |
| ATAA | 0.333 | 0.439 | 0.004 | 0.083 |
| AAGA | 0.342 | 0.395 | 0.004 | 0.083 |
| GGCA | 0.729 | 0.658 | 0.005 | 0.087 |
| CTAG | 0.331 | 0.393 | 0.006 | 0.1 |
| GCTC | 0.352 | 0.31 | 0.007 | 0.1 |
| CCCA | 1.944 | 1.695 | 0.007 | 0.1 |
| CTGG | 0.422 | 0.545 | 0.007 | 0.1 |
| GACC | 0.238 | 0.214 | 0.008 | 0.1 |
| CCTA | 0.9 | 0.799 | 0.009 | 0.1 |
| GTAG | 0.148 | 0.184 | 0.009 | 0.1 |
| AAGG | 0.231 | 0.271 | 0.009 | 0.1 |
| CCTG | 1.665 | 1.49 | 0.01 | 0.1 |
| GGCC | 0.405 | 0.375 | 0.01 | 0.1 |
| ATAT | 0.307 | 0.373 | 0.01 | 0.1 |
| CTAC | 0.243 | 0.275 | 0.011 | 0.107 |
| AGGG | 0.226 | 0.275 | 0.012 | 0.107 |
| ACTT | 0.552 | 0.449 | 0.012 | 0.107 |
| CCTC | 1.124 | 0.997 | 0.012 | 0.107 |
| ATGG | 0.126 | 0.197 | 0.013 | 0.107 |
| ATGC | 0.101 | 0.147 | 0.013 | 0.111 |
| CTGA | 0.594 | 0.671 | 0.014 | 0.112 |
| CCCG | 0.22 | 0.195 | 0.015 | 0.115 |
| CTTG | 0.448 | 0.492 | 0.016 | 0.117 |
| AGAA | 0.604 | 0.69 | 0.017 | 0.117 |
| ATAC | 0.15 | 0.188 | 0.017 | 0.117 |
| ACTC | 0.362 | 0.31 | 0.017 | 0.117 |
| ATGA | 0.185 | 0.256 | 0.018 | 0.121 |
| TACT | 0.484 | 0.453 | 0.02 | 0.13 |
| CTGC | 0.325 | 0.391 | 0.02 | 0.13 |
| CTCA | 0.449 | 0.515 | 0.023 | 0.146 |
| CTAA | 0.529 | 0.587 | 0.025 | 0.15 |
| CCCT | 1.182 | 1.03 | 0.026 | 0.15 |
| ACCT | 0.54 | 0.448 | 0.026 | 0.15 |
| CTAT | 0.415 | 0.456 | 0.027 | 0.15 |
| GTGA | 0.202 | 0.246 | 0.028 | 0.15 |
| AGGA | 0.33 | 0.41 | 0.028 | 0.15 |
| AGGT | 0.192 | 0.236 | 0.029 | 0.15 |
| ATGT | 0.164 | 0.231 | 0.029 | 0.15 |
| AGAC | 0.26 | 0.283 | 0.03 | 0.15 |
| GCAC | 0.295 | 0.246 | 0.03 | 0.15 |
| GTGC | 0.106 | 0.134 | 0.03 | 0.15 |
| TTAC | 0.151 | 0.187 | 0.032 | 0.151 |
| ACTA | 0.377 | 0.334 | 0.032 | 0.151 |
| ACGG | 0.015 | 0.02 | 0.033 | 0.152 |
| GCCT | 0.737 | 0.627 | 0.033 | 0.152 |
| CCTT | 1.401 | 1.23 | 0.035 | 0.155 |
| TCGA | 0.021 | 0.025 | 0.035 | 0.155 |
| TCGG | 0.014 | 0.018 | 0.037 | 0.158 |
| CTCG | 0.047 | 0.058 | 0.037 | 0.159 |
| CGCA | 0.068 | 0.062 | 0.038 | 0.159 |
| GACA | 0.445 | 0.408 | 0.039 | 0.159 |
| ACTG | 0.531 | 0.475 | 0.04 | 0.159 |
| TTAT | 0.334 | 0.412 | 0.04 | 0.159 |
| TACC | 0.33 | 0.305 | 0.042 | 0.164 |
| ACCA | 0.589 | 0.526 | 0.043 | 0.166 |
| TTTC | 0.261 | 0.281 | 0.044 | 0.166 |
| TATA | 0.782 | 0.723 | 0.045 | 0.166 |
| GCAG | 0.407 | 0.334 | 0.045 | 0.166 |
| AAGT | 0.203 | 0.228 | 0.048 | 0.173 |
| GCTT | 0.535 | 0.47 | 0.048 | 0.173 |
| GACT | 0.322 | 0.29 | 0.049 | 0.173 |
| GTGG | 0.126 | 0.176 | 0.05 | 0.176 |
| CCCC | 0.72 | 0.652 | 0.051 | 0.176 |
| TTAA | 0.319 | 0.39 | 0.052 | 0.178 |
| GTAA | 0.229 | 0.265 | 0.053 | 0.178 |
| GCCA | 0.769 | 0.67 | 0.054 | 0.178 |
| ATCA | 0.178 | 0.23 | 0.055 | 0.179 |
| GCCC | 0.383 | 0.33 | 0.058 | 0.181 |
| AAAA | 0.983 | 1.084 | 0.059 | 0.181 |
| TCGT | 0.019 | 0.024 | 0.059 | 0.181 |
| GTGT | 0.171 | 0.215 | 0.059 | 0.181 |
| CGAG | 0.125 | 0.115 | 0.059 | 0.181 |
| ATTG | 0.15 | 0.186 | 0.059 | 0.181 |
| AGAG | 0.485 | 0.544 | 0.062 | 0.186 |
| CACT | 0.584 | 0.541 | 0.066 | 0.195 |
| TATT | 0.928 | 0.89 | 0.068 | 0.195 |
| ACAC | 0.357 | 0.323 | 0.068 | 0.195 |
| CGTA | 0.084 | 0.075 | 0.068 | 0.195 |
| CCGA | 0.052 | 0.059 | 0.069 | 0.195 |
| GCTA | 0.416 | 0.381 | 0.069 | 0.195 |
| CCAC | 0.945 | 0.857 | 0.071 | 0.198 |
| CCAT | 1.296 | 1.19 | 0.074 | 0.203 |
| ACAT | 0.581 | 0.524 | 0.075 | 0.203 |
| ACGA | 0.018 | 0.024 | 0.076 | 0.204 |
| ACGT | 0.023 | 0.03 | 0.077 | 0.204 |
| GTAC | 0.107 | 0.12 | 0.077 | 0.204 |
| CCAG | 1.439 | 1.33 | 0.078 | 0.204 |
| TATC | 0.446 | 0.426 | 0.079 | 0.205 |
| CATT | 0.85 | 0.782 | 0.085 | 0.215 |
| CGCT | 0.078 | 0.074 | 0.085 | 0.215 |
| GCAA | 0.42 | 0.38 | 0.088 | 0.219 |
| GGAC | 0.352 | 0.337 | 0.088 | 0.219 |
| GGAT | 0.526 | 0.502 | 0.089 | 0.219 |
| CTCC | 0.276 | 0.325 | 0.093 | 0.223 |
| AGGC | 0.225 | 0.277 | 0.093 | 0.223 |
| ATCG | 0.015 | 0.019 | 0.093 | 0.223 |
| TCTC | 0.589 | 0.552 | 0.096 | 0.228 |
| TACA | 0.652 | 0.608 | 0.097 | 0.228 |
| CCGT | 0.06 | 0.067 | 0.1 | 0.234 |
| GCAT | 0.446 | 0.401 | 0.102 | 0.236 |
| TCGC | 0.013 | 0.015 | 0.105 | 0.24 |
| ACAG | 0.547 | 0.51 | 0.108 | 0.243 |
| TTGT | 0.217 | 0.261 | 0.108 | 0.243 |
| GCTG | 0.567 | 0.509 | 0.112 | 0.248 |
| ACCC | 0.342 | 0.289 | 0.113 | 0.248 |
| GAGG | 0.26 | 0.293 | 0.113 | 0.248 |
| CAGA | 0.669 | 0.727 | 0.115 | 0.251 |
| CAGT | 0.462 | 0.485 | 0.119 | 0.257 |
| TGGA | 0.533 | 0.617 | 0.122 | 0.26 |
| ACGC | 0.013 | 0.017 | 0.128 | 0.271 |
| TATG | 0.566 | 0.53 | 0.131 | 0.276 |
| TTGG | 0.125 | 0.152 | 0.136 | 0.284 |
| TGGT | 0.39 | 0.437 | 0.14 | 0.288 |
| GACG | 0.036 | 0.033 | 0.144 | 0.293 |
| CAGG | 0.65 | 0.71 | 0.144 | 0.293 |
| TTAG | 0.133 | 0.161 | 0.146 | 0.294 |
| GTTG | 0.207 | 0.24 | 0.151 | 0.299 |
| CATA | 0.623 | 0.595 | 0.151 | 0.299 |
| CAAA | 1.065 | 1.111 | 0.155 | 0.305 |
| GTCG | 0.014 | 0.017 | 0.158 | 0.308 |
| CGCC | 0.094 | 0.09 | 0.16 | 0.31 |
| ATCC | 0.107 | 0.132 | 0.163 | 0.312 |
| TCTT | 0.724 | 0.695 | 0.163 | 0.312 |
| CACC | 0.441 | 0.421 | 0.166 | 0.315 |
| GCGT | 0.019 | 0.023 | 0.169 | 0.315 |
| TTGC | 0.122 | 0.142 | 0.17 | 0.315 |
| CGTT | 0.109 | 0.103 | 0.17 | 0.315 |
| AACC | 0.219 | 0.203 | 0.174 | 0.321 |
| AGAT | 0.391 | 0.402 | 0.176 | 0.321 |
| CATC | 0.501 | 0.474 | 0.177 | 0.321 |
| CACA | 0.836 | 0.801 | 0.179 | 0.322 |
| CAGC | 0.424 | 0.445 | 0.189 | 0.335 |
| CTCT | 0.432 | 0.473 | 0.193 | 0.335 |
| CGTC | 0.088 | 0.085 | 0.193 | 0.335 |
| TGTG | 0.805 | 0.764 | 0.193 | 0.335 |
| TGGG | 0.529 | 0.593 | 0.194 | 0.335 |
| GTAT | 0.236 | 0.258 | 0.194 | 0.335 |
| AGCA | 0.408 | 0.419 | 0.195 | 0.335 |
| CGTG | 0.178 | 0.167 | 0.197 | 0.336 |
| TTGA | 0.227 | 0.262 | 0.198 | 0.337 |
| TGAA | 0.857 | 0.927 | 0.2 | 0.337 |
| CCGG | 0.056 | 0.061 | 0.211 | 0.353 |
| ACAA | 0.578 | 0.553 | 0.216 | 0.359 |
| GCGC | 0.013 | 0.015 | 0.219 | 0.361 |
| GCGA | 0.015 | 0.017 | 0.223 | 0.365 |
| GAGA | 0.328 | 0.352 | 0.228 | 0.372 |
| AACA | 0.483 | 0.46 | 0.231 | 0.373 |
| GGAG | 0.831 | 0.793 | 0.235 | 0.378 |
| ATTA | 0.237 | 0.261 | 0.236 | 0.378 |
| CCAA | 1.189 | 1.131 | 0.241 | 0.384 |
| GAAC | 0.305 | 0.295 | 0.244 | 0.386 |
| TTTA | 0.388 | 0.408 | 0.248 | 0.389 |
| AGCT | 0.341 | 0.327 | 0.25 | 0.39 |
| TGCA | 0.599 | 0.576 | 0.255 | 0.393 |
| TGGC | 0.383 | 0.416 | 0.257 | 0.393 |
| GCGG | 0.01 | 0.012 | 0.258 | 0.393 |
| TCTG | 0.512 | 0.478 | 0.259 | 0.393 |
| AACT | 0.297 | 0.279 | 0.261 | 0.393 |
| TCTA | 0.448 | 0.429 | 0.261 | 0.393 |
| TAGG | 0.278 | 0.302 | 0.27 | 0.404 |
| ATCT | 0.194 | 0.218 | 0.274 | 0.407 |
| GGCG | 0.11 | 0.102 | 0.275 | 0.407 |
| GTCA | 0.19 | 0.212 | 0.283 | 0.416 |
| TGTA | 0.671 | 0.642 | 0.287 | 0.419 |
| ATTC | 0.183 | 0.198 | 0.309 | 0.45 |
| TAGA | 0.391 | 0.419 | 0.314 | 0.452 |
| CAAG | 0.738 | 0.755 | 0.314 | 0.452 |
| TAAA | 0.926 | 0.968 | 0.317 | 0.453 |
| TCCG | 0.027 | 0.029 | 0.329 | 0.468 |
| AGTT | 0.376 | 0.356 | 0.331 | 0.468 |
| TGAT | 0.683 | 0.71 | 0.339 | 0.477 |
| GTCC | 0.103 | 0.116 | 0.35 | 0.49 |
| AAAT | 0.718 | 0.741 | 0.376 | 0.523 |
| TGAC | 0.421 | 0.44 | 0.385 | 0.533 |
| TCAA | 0.432 | 0.449 | 0.4 | 0.548 |
| GGGC | 0.302 | 0.292 | 0.401 | 0.548 |
| GATT | 0.382 | 0.361 | 0.424 | 0.577 |
| TACG | 0.036 | 0.034 | 0.427 | 0.578 |
| CAAT | 0.691 | 0.673 | 0.453 | 0.611 |
| AATT | 0.374 | 0.356 | 0.456 | 0.611 |
| CTTC | 0.449 | 0.463 | 0.462 | 0.617 |
| TAAC | 0.383 | 0.374 | 0.479 | 0.636 |
| AACG | 0.034 | 0.035 | 0.486 | 0.642 |
| CACG | 0.1 | 0.098 | 0.493 | 0.648 |
| TTTT | 0.483 | 0.521 | 0.497 | 0.649 |
| TGTC | 0.559 | 0.549 | 0.506 | 0.658 |
| TAAT | 0.751 | 0.763 | 0.51 | 0.658 |
| TCAT | 0.545 | 0.556 | 0.512 | 0.658 |
| AGCC | 0.28 | 0.286 | 0.521 | 0.667 |
| CGAT | 0.081 | 0.078 | 0.523 | 0.667 |
| CCGC | 0.05 | 0.052 | 0.531 | 0.673 |
| TGAG | 0.762 | 0.788 | 0.554 | 0.698 |
| TAGC | 0.227 | 0.235 | 0.56 | 0.703 |
| GGTT | 0.547 | 0.534 | 0.567 | 0.708 |
| TCCT | 0.604 | 0.588 | 0.576 | 0.716 |
| TGTT | 0.913 | 0.901 | 0.587 | 0.723 |
| CGGA | 0.06 | 0.062 | 0.59 | 0.723 |
| AATC | 0.211 | 0.204 | 0.59 | 0.723 |
| CTTA | 0.512 | 0.519 | 0.598 | 0.723 |
| GATG | 0.357 | 0.377 | 0.599 | 0.723 |
| GTCT | 0.235 | 0.248 | 0.604 | 0.723 |
| CGAC | 0.049 | 0.048 | 0.605 | 0.723 |
| AGTG | 0.44 | 0.462 | 0.605 | 0.723 |
| GGAA | 0.815 | 0.804 | 0.613 | 0.73 |
| AGTC | 0.255 | 0.265 | 0.623 | 0.738 |
| GAGT | 0.165 | 0.17 | 0.625 | 0.738 |
| GGGT | 0.262 | 0.257 | 0.633 | 0.739 |
| GTTT | 0.501 | 0.478 | 0.633 | 0.739 |
| CGGT | 0.05 | 0.051 | 0.637 | 0.739 |
| GAAA | 0.734 | 0.747 | 0.638 | 0.739 |
| TCCA | 0.53 | 0.539 | 0.643 | 0.742 |
| GTTA | 0.277 | 0.284 | 0.651 | 0.748 |
| TGCG | 0.041 | 0.04 | 0.674 | 0.771 |
| AGCG | 0.044 | 0.045 | 0.68 | 0.773 |
| TGCT | 0.595 | 0.589 | 0.694 | 0.786 |
| GAGC | 0.173 | 0.177 | 0.712 | 0.803 |
| AGTA | 0.327 | 0.334 | 0.716 | 0.804 |
| TGCC | 0.441 | 0.436 | 0.734 | 0.82 |
| CGGC | 0.075 | 0.074 | 0.751 | 0.836 |
| GGGG | 0.342 | 0.346 | 0.763 | 0.845 |
| GAAT | 0.516 | 0.511 | 0.774 | 0.851 |
| GGTC | 0.379 | 0.386 | 0.775 | 0.851 |
| GGGA | 0.53 | 0.535 | 0.78 | 0.853 |
| TCAC | 0.384 | 0.379 | 0.783 | 0.853 |
| CGCG | 0.017 | 0.016 | 0.794 | 0.856 |
| CAAC | 0.412 | 0.414 | 0.796 | 0.856 |
| CATG | 0.726 | 0.719 | 0.796 | 0.856 |
| CTTT | 0.853 | 0.839 | 0.807 | 0.864 |
| AATG | 0.36 | 0.365 | 0.814 | 0.868 |
| TTTG | 0.202 | 0.204 | 0.834 | 0.882 |
| CGAA | 0.094 | 0.093 | 0.837 | 0.882 |
| GATC | 0.224 | 0.22 | 0.837 | 0.882 |
| CGGG | 0.087 | 0.087 | 0.86 | 0.9 |
| GGTA | 0.47 | 0.475 | 0.862 | 0.9 |
| GAAG | 0.512 | 0.509 | 0.885 | 0.92 |
| TAGT | 0.277 | 0.274 | 0.894 | 0.92 |
| TCAG | 0.384 | 0.389 | 0.895 | 0.92 |
| AAAC | 0.401 | 0.402 | 0.895 | 0.92 |
| TAAG | 0.463 | 0.466 | 0.905 | 0.926 |
| GTTC | 0.214 | 0.216 | 0.916 | 0.934 |
| ATTT | 0.44 | 0.442 | 0.963 | 0.974 |
| GATA | 0.306 | 0.307 | 0.968 | 0.974 |
| AATA | 0.393 | 0.392 | 0.97 | 0.974 |
| GGTG | 0.708 | 0.71 | 0.97 | 0.974 |
| TCCC | 0.439 | 0.439 | 0.989 | 0.989 |

### Example 6: Testing the Lower Limits of Detection

In order to test the lower limit of cell of origin detection, we performed a series of mixture experiments where we mixed plasma DNA from a metastatic colorectal cancer patient with plasma DNA from a pool of 11 healthy individuals. We used the same multi cell type deconvolution described above to determine cell types. While the healthy pool (“healthymix”) had almost no epithelial cell present, the CRC case (“PL5655_CRC”) was estimated to be 63% epithelial cells (FIG. 15). With a mixture of half CRC and half healthy cfDNA (“mix50”), the epithelial content was 39%, and with a mixture of one quarter CRC (“mix25”), the epithelial content was 17%. Since these mixtures approximately matched a proportional reduction in epithelial cells as expected, we tried more diluted mixtures-⅛^{th} (mix 12.5), 1/16^{th} (mix6.25), and 1/32 (mix3.125). Epithelial cells were detected down to the most diluted mixture of 1/32 or 3.125%. Each of these samples had similar numbers of reads as our earlier lung adenocarcinoma study (between 3-5M, Table 4). Thus, we believe shallow Nanopore whole-genome sequencing has the power to resolve tumor fractions less than 5%. With improved deconvolution methods based on a whole genome methylation atlas, we expect detection of even lower tumor fractions.

TABLE 4
cfNano CRC vs. healthy plasma mixture samples from
Hadassah Hebrew University Medical Center, processed
using 5mC + 5hmC modification model.

| sample ID | sample | mapped reads |
|---|---|---|
| HU004.02 | Hadassah CRC | 4,696,475 |
| HU004.03 | Hadassah BRCA | 4,243,313 |
| HU005.10 | HUJI healthy 1 | 2,035,125 |
| HU005.11 | HUJI healthy 2 | 2,360,434 |
| HU005.12 | HUJI healthy 3 | 1,573,544 |
| HU008.22 | Hadassah CRC PL5655 | 812,851 |
| HU008.23 | Hadassah CRC PL5832 | 780,407 |
| HU008.24 | Hadassah CRC PL5761 | 870,472 |
| HU012.01 | Hadassah healthy 1 | 731,383 |
| HU012.02 | Hadassah healthy 2 | 1,073,751 |
| HU012.03 | Hadassah healthy 3 | 710,218 |
| HU012.04 | Hadassah healthy 4 | 1,778,189 |
| HU012.05 | Hadassah healthy 5 | 1,306,767 |
| HU012.06 | Hadassah healthy 6 | 1,567,810 |
| HU012.07 | Hadassah healthy 7 | 941,445 |
| HU012.08 | Hadassah healthy 8 | 939,762 |
| HU012.09 | Hadassah healthy 9 | 1,030,852 |
| HU012.10 | Hadassah healthy 10 | 845,033 |
| HU012.11 | Hadassah healthy 11 | 583,662 |
| HU013.01 | Hadassah healthymix | 3,664,862 |
| HU013.02 | Hadassah PL5655 | 3,510,503 |
| HU013.03 | mix25 | 5,120,900 |
| HU016.04 | mix50 | 3,059,381 |
| HU016.05 | mix12.5 | 4,101,627 |
| HU016.06 | mix6.25 | 4,179,725 |
| HU016.07 | mix3.125 | 3,171,970 |

### Example 7: Detection of Targetable Genomic Amplifications Using Multiple Genomic Features

We used cfNano to sequence plasma cfDNA from one metastatic colorectal cancer case (HU004.02) and one breast cancer case (HU004.03) from Hadassah Hebrew University Medical Center, both of which were HER2 positive based on clinical testing of tumor samples. The high level amplification of the ERBB2 gene, which encodes HER2, can be clearly seen on the copy number alteration plots using ichorCNA (FIG. 16A). The precise extent of the genomic amplification can be seen by zooming in on the ERBB2 locus in the Integrated Genome Viewer (IGV), and showing the read coverage tracks (FIG. 16B). These samples have 4.7M and 4.2M mapped reads, respectively. Thus, cfNano using a relatively shallow read coverage could be used to track ERBB2 amplifications at diagnosis and throughout treatment with standard neoadjuvant and targeted therapies, and to guide treatment using several available ERBB2 targeted therapies.

Because integration of multiple genomic features could improve our detection of clinically actionable genomic amplifications, we investigated whether a fragmentation feature associated with cancer DNA would be enriched at the amplified region, where a relatively higher proportion of total DNA is derived from cancer. To show this, we plotted the density of fragmentation lengths for bins with equally sized bins containing 5,000 fragments across chromosome 17, for the ERBB2-amplified colorectal cancer case HU004.02 (FIG. 17). Three of these bins overlapped the ERBB2 amplification, and all three showed an overabundance of fragments between 125-150 bp, and the lack of a clear peak at 167 bp. A similar density plot was observed at two bins overlapping an amplification at chr17q11.2. In contrast, most other bins on chromosome 17 showed a strong peak at around 167 bp. This shows that copy number can be combined with other cancer-associated features to improve identification of copy number altered regions.

### Example 8: Detecting DNA by Cancer-Specific Differences in 5-Hydroxymethylation

We used the combined 5-methylcytosine (5mC) and 5-hydroxymethylcytosine (5hmC) modification caller from Oxford Nanopore to call both of these modifications on a set of plasma cfDNA samples from the Oncology Department of the Hadassah Hebrew University Medical Center. This set included 15 samples from healthy individuals (who had been screened for cancer in the Oncology department and tested negative) and 5 samples from metastatic colorectal cancer patients. All were sequenced using shallow Nanopore whole-genome sequencing as described earlier, each with between 1-5 million raw sequence reads (Table 4).

We first sought to verify that the 5hmC patterns were similar to those using independent sequencing technologies. For this, we used CTCF binding sites, which do not vary between different cell types. Because the regions surrounding CTCF binding sites reveal nucleosome positions in both the 5mC and 5hmC signal, they have been used as key landmarks to validate new single nucleotide resolution sequencing methods. The first single-nucleotide method to determine 5hmC patterns is TET-assisted Bisulfite sequencing or TAB-seq. TAB-seq showed that 5mC was strongly demethylated from −200 bp upstream of the CTCF motif to 200 bp downstream, whereas 5hmC had two methylation peaks within this central region. 5mC and 5hmC showed similar patterns of phased nucleosomes at −600 bp to −200 bp upstream, to 200 bp to 600 bp downstream. Newer sequencing methods have been developed which replace bisulfite conversion with enzymatic conversion. One of the most popular methods, Enzymatic Methyl-seq (EM-seq) uses the APOBEC3A enzyme. This method found the same 5mC and 5hmC patterns at CTCF binding sites as TAB-seq did. When we plotted 5mC and 5hmC percentages for our 20 Nanopore plasma cfDNA samples, we observed patterns that matched these earlier studies using established sequencing technologies (as compared to Miao Yu et al., “Base-Resolution Analysis of 5-Hydroxymethylcytosine in the Mammalian Genome”, Cell 149, no. 6, (Elsevier BV, 2012) and Vladimir B. Teif et al., “Nucleosome Repositioning Links DNA (de) Methylation and Differential CTCF Binding during Stem Cell Development”, Genome Research 24, no. 8 (Cold Spring Harbor Laboratory, 2014), and Zhiyi Sun et al., “Nondestructive Enzymatic Deamination Enables Single-Molecule Long-Read Amplicon Sequencing for the Determination of 5-Methylcytosine and 5-Hydroxymethylcytosine at Single-Base Resolution”, Genome Research 31, no. 2 (Cold Spring Harbor Laboratory, 2021), herein incorporated by reference in their entirety). Specifically, both 5mC and 5hmC had the same nucleosomal phasing pattern for regions more than 200 bp away from the CTCF binding site, but the two cytosine modifications had divergent patterns within the central region from −200 bp upstream to 200 bp downstream of the binding site-5mC was fully unmethylated, while 5hmC was methylated. This was consistent with all earlier studies using TAB-seq and EM-seq.

Having established that the combined 5mC and 5hmC modification calling of Nanopore cfDNA samples accurately reproduced data generated using multiple independent and well-established techniques, we went on to look for regions that could differentiate cancer from healthy plasma cfDNA samples. Unfortunately, no cancer cfDNA studies have used single-nucleotide resolution approaches such as TAB-seq or EM-seq to identify differences in 5hmC. However, a popular approach based on enrichment of 5hmC modified regions using immunoprecipitation (hMe-Seal) showed that seven cancer types could be detected from cfDNA based on their genome-wide 5hmC patterns. This has subsequently been validated for other cancer types such as esophageal and pancreatic cancer. While the data from these enrichment approaches is not directly comparable to single nucleotide resolution data such as Nanopore, we looked for regions of interest that might be used to analyze cancer-associated signals in our Nanopore 5hmC analysis. Studies of plasma cfDNA in non-small cell lung cancer and hepatocellular carcinoma indicated that increased 5hmC at active promoters and other active regulatory sites was associated with cancer samples relative to healthy controls, so we decided to investigate these regions in our cfDNA Nanopore data.

Based on the previously observed increased 5hmC at active promoters, we investigated 5hmC levels in our Nanopore samples at a set of ubiquitously active CpG Island promoters from Kelly et al., We used the same 15 healthy Nanopore samples and 5 metastatic colorectal (CRC) samples described previously, plotting average 5mC and 5hmC levels at these active promoters (FIG. 19). In agreement with the earlier studies based on Illumina sequencing, the five CRC Nanopore samples were higher in 5hmC than the 15 healthy Nanopore samples (FIG. 19, right). This hyper-hydroxymethylation appeared to be strongest in the central 1,000 bp region from −500 to +500 relative to the transcription start site (TSS). Unlike the 5hmC pattern, the 5mC pattern was very similar between CRC and healthy samples (FIG. 19, left). This finding suggests that 5hmC at these and other active gene regulatory regions could be used in combination with the other signals described above, to improve detection and characterization of cancer-associated DNA.

### Example 9: Improved Library Cleanup Protocol

The cfNano protocol makes use of a more permissive cleanup step with higher concentrations of SPRI beads and thus the retention of a greater amount of small cfDNA molecules (those below 200 bp). As shown above, these smaller cfDNA molecules are highly useful in cfDNA analyses that make use of 5mC and 5hmC modifications to determine cell type and tissue of origin and cancer origin. However, as the cfDNAs are smaller, the cfDNAs ligated to adapter are smaller. During library preparation the adapter ligated cfDNAs and the unligated adapter need to be separated so that only the adapter ligated cfDNAs are introduced to the nanopore array apparatus. Free adapter will still transduce the nanopores, taking up the available nanopores for sequencing and producing unusable/uninformative reads. This consumes throughput and slows down the sequencing procedure.

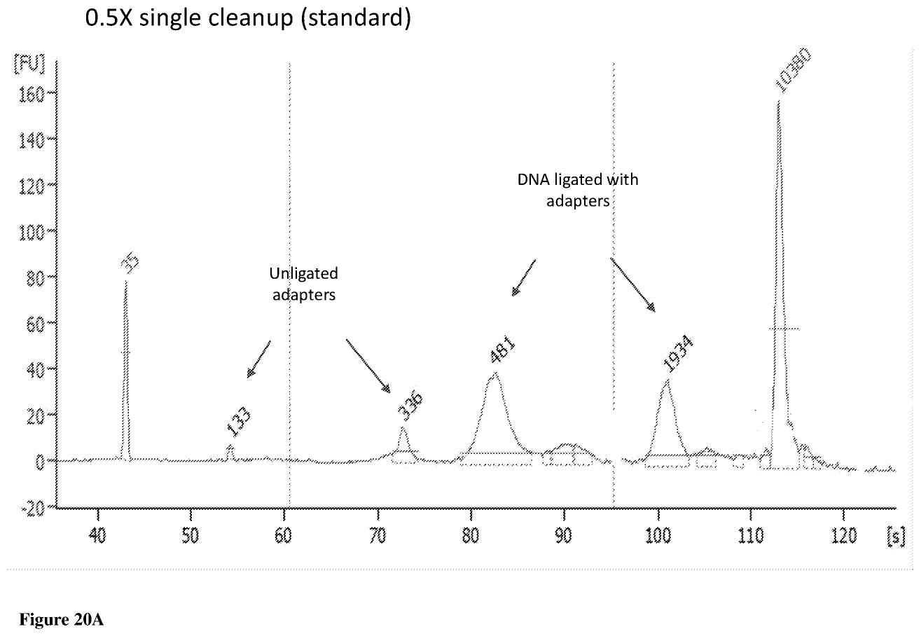

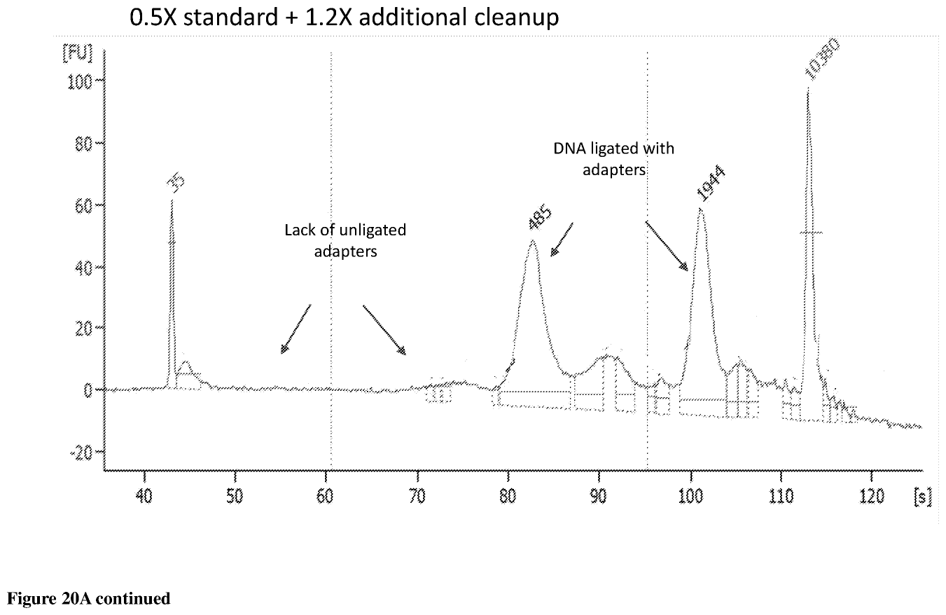

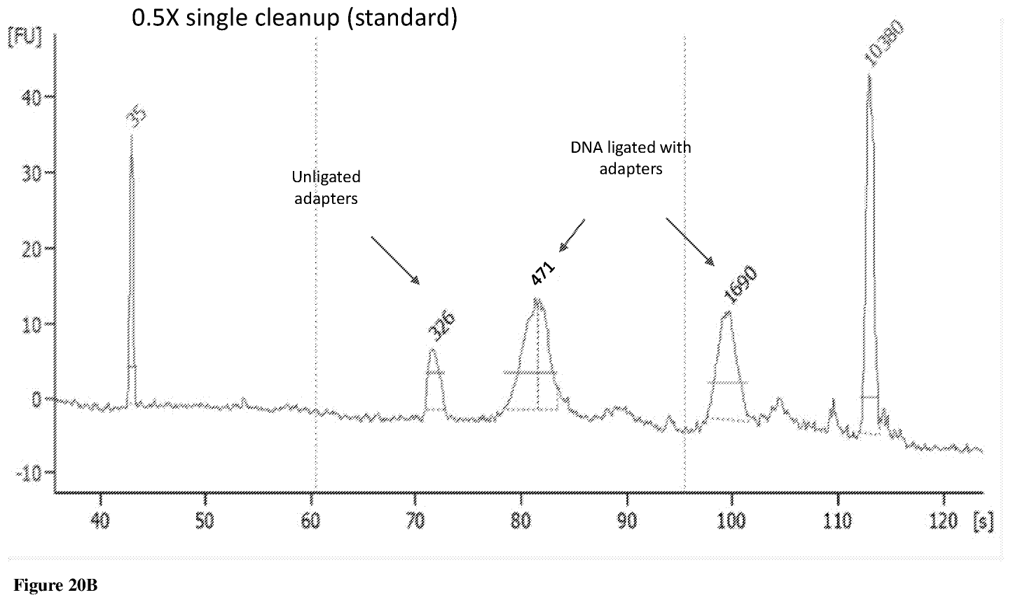

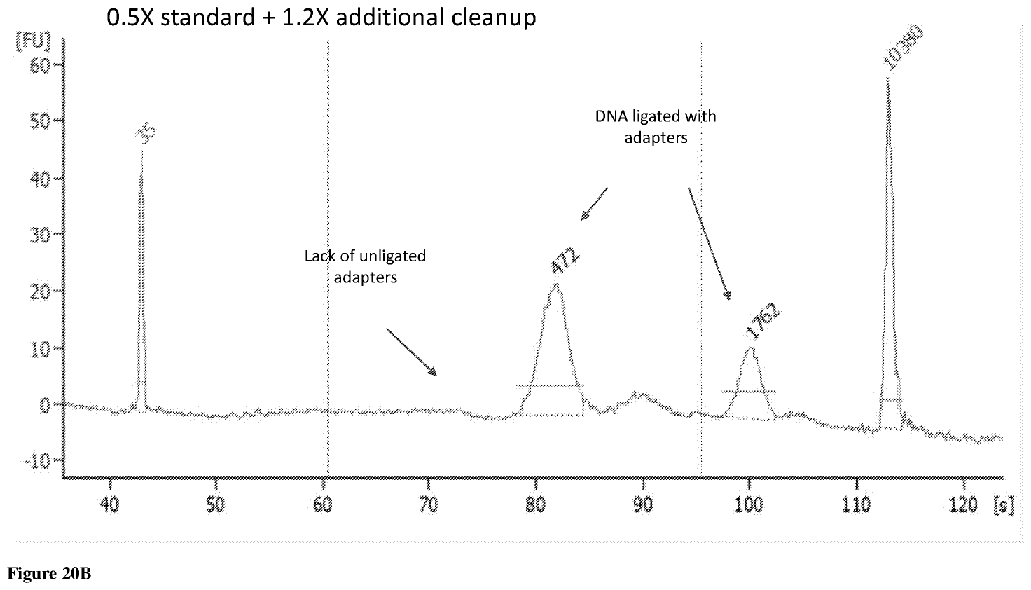

Libraries of cfDNAs were generated using the standard library production protocol which includes a cleanup step of 0.5×SPRI beads to remove unligated adapters. In parallel the same amount of cfDNA sample was used to generate a library using an identical protocol but with a double SPRI cleanup. The same 0.5×SPRI cleanup was followed by a 1.2×SPRI cleanup. This experiment was first performed with a high cfDNA input pool (60 ng). When there is abundant cfDNA unligated adapter is less of a problem as a greater percentage of the molecules in the mix are cfDNA. Even with high input, there was a greater contamination of unligated adapter in the standard protocol as in the double cleanup protocol (FIG. 20A). At low input (16 ng), the contamination of unligated adapter was far greater. This creates a double problem as there will be reduced informative reads due to the low input and increased useless reads from the adapter. The double SPRI cleanup was able to completely remove the unligated adapter even at the lower input (FIG. 20B).

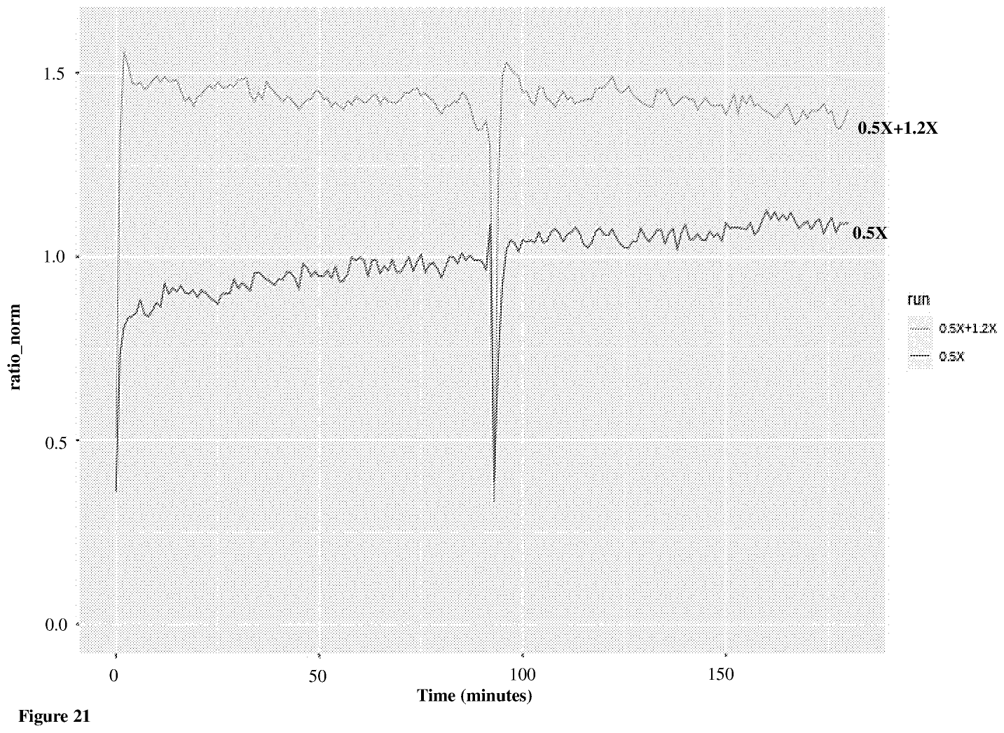

The produced low input libraries were sequenced using a nanopore array as described hereinabove. As expected, the high proportion of unligated adapters negatively affects the yield of the experiment in the first 3 hours, as free adapters occupy pores making them unavailable for sequencing library DNA. For this analysis the total number of pores actively sequencing strands over the total number of occupied pores was calculated. The total occupied pores were defined as pores sequencing a strand (of adapter-ligated DNA), sequencing adapter, unavailable pores (pores currently unavailable for sequencing and recovering) and pores in active feedback state (pore reversing the current in order to eject analyte and unblock itself). For the first 3 hours of the run (the total time analyzed), the double cleanup protocol produced a nearly 50% increase in pores with actual cfDNA strands being sequenced (FIG. 21). This demonstrates the unexpected advantage produced by using a two-step cleanup. This extra cleanup is especially advantageous when the initial amount of input DNA is low, which is common when analyzing cfDNA samples.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of determining a tissue of origin, a cell type of origin, origination from a cancerous cell, or a combination thereof of cell-free DNA (cfDNA), the method comprising:
a. providing a sample comprising cfDNA, optionally wherein the sample comprises cfDNA enriched for nucleic acids between 50 and 200 nucleotides in length to produce enriched cfDNA;
b. passing said cfDNA or said enriched cfDNA through a nanopore sequencer to produce a sequence of said cfDNA wherein said sequence comprises DNA modification data selected from: methylation data, hydroxymethylation data and both; and
c. identifying for said cfDNA or said enriched cfDNA passed through a nanopore a tissue of origin, cell type of origin, origination from a cancerous cell or a combination thereof based on said sequence comprising DNA modification data;
thereby determining a tissue of origin, cell type of origin, origination from a cancerous cell or a combination thereof of cfDNA.

2. (canceled)

3. The method of claim 1, wherein said providing comprises providing a sample from a subject and extracting cfDNA from said sample, optionally wherein said sample is a bodily fluid, optionally wherein said bodily fluid is blood.

4. (canceled)

5. The method of claim 1, wherein said cfDNA is unamplified after it is extracted from a sample from a subject.

6. The method of claim 1, wherein said cfDNA has been modified with a sequencing adapter and optionally a nucleic acid barcode that uniquely identifies a sample from which comes the cfDNA.

7. The method of claim 3, wherein said providing further comprises employing SPRI bead size exclusion to remove DNA of a size below 50 nucleotides while retaining cfDNA of a size between 50 nucleotides and 200 nucleotides, optionally wherein said SPRI bead size exclusion comprises an SPRI bead to sample ratio of about 1.8:1 by volume.

8. (canceled)

9. The method of claim 1, wherein enriched is as compared to cfDNA that has undergone SPRI bead size exclusion comprising an SPRI bead to sample ratio of about 0.5:1 by volume.

10. The method of claim 1, wherein said nanopore sequencer is a capable of single base pair sequencing resolution and can distinguish between methylated DNA bases, hydroxymethylated DNA bases and unmethylated/unhydroxymethylated DNA bases.

11. The method of claim 10, wherein said nanopore sequencer comprises an alpha-hemolysin protein pore through which said cfDNA translocates, optionally wherein said nanopore sequencer is an Oxford Nanopore sequencer.

12. (canceled)

13. The method of claim 1, wherein said producing a sequence comprises applying a trained machine learning model to an electrical trace produced by said cfDNA as it translocates through said nanopore, optionally wherein said machine learning model is a convolutional neural network (CNN), and training said machine learning model to identify individual bases within said electrical trace, optionally wherein said identifying individual bases comprises identifying modified and unmodified DNA bases.

14-15. (canceled)

16. The method of claim 1, wherein identification of a modified or unmodified DNA base at an informative genetic locus indicates the tissue or cell type of origin of the cfDNA, optionally wherein the informative genetic locus indicates the cfDNA is from a cancerous cell and/or indicates the tissue or cell type of the cancerous cell.

17-18. (canceled)

19. The method of claim 1, wherein a plurality of cfDNA molecules from the same source is provided and passed and identification of an average hypomethylation on said cfDNA molecules as compared to control cfDNA molecules indicates the hypomethylated cfDNA is from cancerous cells, optionally wherein said control cfDNA molecules are from a subject that does not suffer from cancer.

20. (canceled)

21. The method of claim 1, wherein said method is a method of determining origination from a cancerous cell and further comprises identifying a cancer-specific DNA modification change in said cancerous cell.

22. The method of claim 1, wherein a plurality of cfDNA molecules from the same source is provided and passed and wherein said produced sequence has an average of at least 0.15 uniquely aligned reads covering each base in the genome or at least 2 million uniquely aligned reads total.

23. (canceled)

24. The method of claim 1, further comprising performing a copy number analysis on said cfDNA after said passing and wherein said identifying is based on said sequence comprising DNA modification data and said copy number analysis.

25. The method of claim 1, wherein said DNA methylation is 5-methylcytosine (5mC) methylation and said hydroxymethylation is 5-hydroxymethylcytosine (5hmC) hydroxymethylation.

26. The method of claim 6, where said cfDNA has been ligated to said sequencing adapter and further comprising performing an SPRI bead cleanup step to remove unligated sequencing adapter from said cfDNA modified with a sequencing adapter, and wherein said cleanup step comprises a first SPRI bead size exclusion comprising an SPRI bead to sample ratio of about 0.5:1 by volume and a second SPRI bead size exclusion comprising an SPRI bead to sample ratio of about 1.2:1 by volume.

27. A method of determining a tissue of origin, cell type of origin, origination from a cancerous cell or a combination thereof of cell-free DNA (cfDNA), the method comprising:
a. providing a sample comprising cfDNA and enriched for nucleic acids between 50 and 200 nucleotides in length;
b. passing said cfDNA through a nanopore sequencer to produce a sequence of said cfDNA;
c. performing a fragmentation analysis on said cfDNA after said passing, optionally wherein said fragmentation analysis comprises fragment length analysis, fragmentation locational analysis, fragmentation-based nucleosome detection, fragment pattern analysis, fragment end motif analysis, fragment jagged end analysis, fragmentation-based DNA-binding protein binding analysis or a combination thereof; and
d. identifying for said passed cfDNA a tissue of origin, cell type of origin, origination from a cancerous cell or a combination thereof based on said sequence and said fragmentation analysis;
thereby determining a tissue of origin, cell type of origin, origination from a cancerous cell or a combination thereof of cfDNA.

28. (canceled)

29. The method of claim 27, further comprising performing a copy number analysis on said cfDNA after said passing and wherein said identifying is based on said sequence said fragmentation analysis and said copy number analysis, optionally wherein said copy number analysis results in the detection of an oncogene amplification and further comprising administering an agent that targets said oncogene.

30-32. (canceled)

33. The method of claim 1, further comprising treating a subject that provided said cfDNA with a suitable treatment for cancer based on determining a tissue of origin, cell type of origin, origination from a cancerous cell, or a combination thereof of said cfDNA.

34. A method of producing an adapter ligated cfDNA library for analysis with a nanopore apparatus, the method comprising:
a. providing a sample comprising cfDNA;
b. ligating a short adapter below 75 nucleotides in length to said cfDNA to produce adapter ligated cfDNA;
c. removing unligated adapter from said adapter ligated cfDNA by a cleanup step comprises a first SPRI bead size exclusion comprising an SPRI bead to sample ratio of about 0.5:1 by volume and a second SPRI bead size exclusion comprising an SPRI bead to sample ratio of about 1.2:1 by volume;
thereby producing an adapter ligated cfDNA library for analysis with a nanopore apparatus.

35-37. (canceled)
